(19) United States
(12) Patent Application Publication
(10) Pub. No.: US 2014/0077179 A1
(43) Pub. Date: Mar. 20, 2014

(54) NOVEL COMPOUND FOR ORGANIC ELECTRONIC MATERIAL AND ORGANIC ELECTROLUMINESCENT DEVICE USING THE SAME

(71) Applicants: Hyo Nim Shin, Gueonggi-do (KR); Chi Sik Kim, Gyeonggi-do (KR); Su Hyun Lee, Gyeonggi-do (KR); Hong Yoep Na, Seoul (KR); Seok-Keun Yoon, Gyeonggi-do (KR); Soo Jin Yang, Gyeonggi-do (KR); Soo Yong Lee, Chungcheongnam-do (KR); Young Jun Cho, Gyeonggi-do (KR); Kyoung-Jin Park, Gyeonggi-do (KR); Kyung Joo Lee, Seoul (KR); Bong Ok Kim, Seoul (KR)

(72) Inventors: Hyo Nim Shin, Gueonggi-do (KR); Chi Sik Kim, Gyeonggi-do (KR); Su Hyun Lee, Gyeonggi-do (KR); Hong Yoep Na, Seoul (KR); Seok-Keun Yoon, Gyeonggi-do (KR); Soo Jin Yang, Gyeonggi-do (KR); Soo Yong Lee, Chungcheongnam-do (KR); Young Jun Cho, Gyeonggi-do (KR); Kyoung-Jin Park, Gyeonggi-do (KR); Kyung Joo Lee, Seoul (KR); Bong Ok Kim, Seoul (KR)

(73) Assignee: Rohm and Haas Electronic Materials Korea Ltd., Chungcheongnam-do (KR)

(21) Appl. No.: 13/885,230

(22) PCT Filed: Nov. 16, 2011
(86) PCT No.: PCT/KR2011/008759

§ 371 Date: Nov. 26, 2013

(30) Foreign Application Priority Data
Nov. 16, 2010 (KR) 10-2010-0113627
Nov. 15, 2011 (KR) 10-2011-0119015

(51) Int. Cl. H01L 51/00; H01L 51/50

(52) U.S. Cl. CPC H01L 51/0067; H01L 51/0072; H01L 51/0094; H01L 51/0071; H01L 51/50
USPC 257/40; 546/276.7; 544/212; 544/225; 544/294; 544/292; 544/283

(57) ABSTRACT

Provided are a novel compound for an organic electronic material and an organic electroluminescent device using the same. The compound for an organic electronic material according to the present invention has high electron transport efficiency, thereby preventing crystallization at the time manufacturing of a device, and allows a layer to be easily formed, thereby improving current characteristics of the device, and thus an OLED device having a lowered driving voltage and improved power efficiency as well as superior luminous efficiency and lifespan characteristics as compared with the existing material can be manufactured.

### FIELD OF THE INVENTION

The present invention relates to a novel compound for an organic electronic material and an organic electroluminescent device using the same.

### BACKGROUND OF THE INVENTION

Among display devices, an electroluminescent device (EL device) is a self-luminescent type display device, and has advantages of a wide viewing angle, excellent contrast, and fast response speed. An organic EL device, which uses aromatic diamine having a low molecular weight, and an aluminum complex, as a material for forming a luminescent layer, was first developed by Eastman Kodak Company [Appl. Phys. Lett. 51, 913, 1987].

A luminescent material is the most important factor for determining luminous efficiency in an OLED. A fluorescent material has been widely used as the luminescent material until now, but development of a phosphorescent material is the best method that can improve the luminous efficiency theoretically up to four times in an electroluminescent mechanism. Further, an iridium (III) complex-based phosphorescent luminescent material has been widely known as the phosphorescent luminescent material until now, and materials such as (acac)Ir(btp)₂ (bis(2-(2′-benzothienyl)-pyridinato-N,C-3′)iridium(acetylacetonate)), Ir(ppy)₃ (tris(2-phenylpyridine)iridium), Firpic (Bis(4,6-difluorophenylpyridinato-N,C2)picolinatoiridium), and the like, have been known for respective RGB colors. In particular, many phosphorescent materials have been recently studied in Korea, Japan, and Europe.

As a host material for a phosphorescent luminescent body, CBP (4,4′-N,N′-dicarbazole-biphenyl) is the most widely known until now, and a high-efficiency OLED to which a hole blocking layer of BCP (Bathocuproine), BAlq (aluminum(III)bis(2-methyl-8-quinolinato)(4-phenylphenolate)), or the like is applied, is known. Also, The Pioneer Company in Japan has developed high-performance OLED by using a BAlq derivative as a host.

The existing materials are advantageous in view of luminescent properties. However, they may be deformed when subjected to a high-temperature depositing process under vacuum, due to a low glass transition temperature and inferior thermal stability thereof. Since the OLED satisfies the relationship, power efficiency=[(π/voltage)×current efficiency] in the OLED, power efficiency is inversely proportional to the voltage. Therefore, power efficiency needs to be raised in order to lower power consumption of the OLED. In fact, an OLED device Using a phosphorescent luminescent material has a significantly higher current efficiency (cd/A) as compared with an OLED device Using a fluorescent luminescent material. However, an OLED where the existing materials such as BAlq or CBP is used as a host of a phosphorescent luminescent material, has a higher driving voltage as compared with the OLED device Using the fluorescent luminescent material, thereby providing no large advantages in view of power efficiency (Im/w). Furthermore, the OLED device using the phosphorescent luminescent material did not provide satisfactory lifespan.

Meanwhile, PCT Publication No. WO/2006/049013 discloses a compound for organic electroluminescent element using a fused ring as backbone. However, the above publication does not teach a compound in which a heteroaromatic ring and an aromatic ring are linked to benzocarbazole.

### Technical Problem

An object of the present invention is to provide a compound for an organic electronic material with an excellent backbone, allowing superior luminous efficiency and a long device lifespan and appropriate color coordinates, as compared with the existing material.

Another object of the present invention is to provide an organic electroluminescent device having high efficiency and long lifespan by employing the compound for an organic electronic material as a luminescent material.

### Technical Solution

The present invention is directed to a compound for an organic electronic material expressed by Chemical Formula 1 below and an organic electroluminescent device using the same, and the compound for an organic electronic material according to the present invention exhibits superior luminous efficiency and lifespan characteristics as compared with the existing material, thereby manufacturing an OLED device having significantly improved driving lifespan as well as improved power consumption due to an increase in power efficiency.

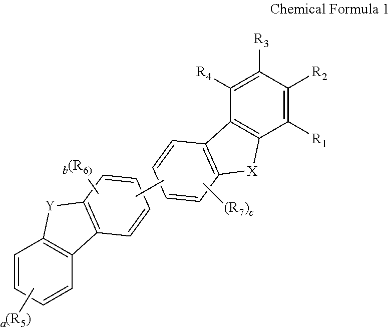

[In Chemical Formula 1, X represents —O—, —S—, —CR₁₁R₁₂— or N-L₁-Ar₁; Y represents —O—, —S—, —CR₁₃R¹⁴— or N-L₂-Ar₂; but Y necessarily represents N-L₁-Ar₁ when X represents —O—, —S— or —CR₁₁R₁₂—; X necessarily represents N-L₂-Ar₂ when Y represents —O—, —S— or —CR₁₃R¹⁴—; one of R₁ through R₄ is linked to an adjacent substituent via

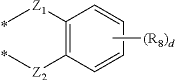

to form a fused ring, the others thereof independently represent hydrogen, deuterium, halogen, substituted or unsubstituted (C1-C30)alkyl, substituted or unsubstituted (C6-C30)aryl, substituted or unsubstituted (C3-C30)heteroaryl, substituted or unsubstituted (C3-C30)cycloalkyl, substituted or unsubstituted 5- to 7-membered heterocycloalkyl, substituted or unsubstituted (C6-C30)ar(C1-C30)alkyl, substituted or unsubstituted (C6-C30)aryl fused with one or more cycloalkyl, 5- to 7-membered heterocycloalkyl fused with one or more substituted or unsubstituted aromatic rings, (C3-C30)cycloalkyl fused with one or more substituted or unsubstituted aromatic rings, —NR₂₁R₂₂, —SiR₂₃R₂₄R₂₅, —SR₂₆, —OR₂₇, (C2-C30)alkenyl, (C2-C30)alkynyl, cyano, nitro, or hydroxy; L₁ and L₂ independently represent a single bond, substituted or unsubstituted (C6-C30)arylene, or substituted or unsubstituted (C3-C30)heteroarylene; Ar₁ and Ar₂ independently represent hydrogen, deuterium, halogen, substituted or unsubstituted (C1-C30)alkyl, substituted or unsubstituted (C6-C30)aryl, or substituted or unsubstituted (C3-C30)heteroaryl; one of Z₁ and Z₂ represents a single bond, and the other thereof represents —O—, —S—, —CR₃₁R₃₂—, —SiR₃₃R₃₄—, or —NR₃₅—; R₅ through R₈ independently represent hydrogen, deuterium, halogen, substituted or unsubstituted (C1-C30)alkyl, substituted or unsubstituted (C6-C30)aryl, substituted or unsubstituted (C3-C30)heteroaryl, substituted or unsubstituted (C3-C30)cycloalkyl, substituted or unsubstituted 5- to 7-membered heterocycloalkyl, substituted or unsubstituted (C6-C30)ar(C1-C30)alkyl, substituted or unsubstituted (C6-C30)aryl fused with one or more (C3-C30)cycloalkyl, 5- to 7-membered heterocycloalkyl fused with one or more substituted or unsubstituted aromatic rings, (C3-C30)cycloalkyl fused with one or more substituted or unsubstituted aromatic rings, —NR₂₁R₂₂, SiR₂₃R₂₄R₂₅, —SR₂₆, —OR₂₇, (C2-C30)alkenyl, (C2-C30)alkynyl, cyano, nitro, or hydroxy; R₁₁ through R₁₄, R₂₁ through R₂₇, and R₃₁ through R₃₅ independently represent hydrogen, deuterium, halogen, substituted or unsubstituted (C1-C30)alkyl, substituted or unsubstituted (C6-C30)aryl, substituted or unsubstituted (C2-C30)heteroaryl, substituted or unsubstituted (C3-C30)cycloalkyl, substituted or unsubstituted 5- to 7-membered heterocycloalkyl, substituted or unsubstituted (C6-C30)ar(C1-C30)alkyl, substituted or unsubstituted (C6-C30)aryl fused with one or more (C3-C30)cycloalkyl, 5- to 7-membered heterocycloalkyl fused with one or more substituted or unsubstituted aromatic rings, or (C3-C30)cycloalkyl fused with one or more substituted or unsubstituted aromatic rings; a through d independently represent an integer of 1 to 4, and they may be the same or different from one another when they independently represent an integer of 2 or more; b and c independently represent an integer of 1 to 3, and they may be the same or different from each other when they independently represent an integer of 2 or more; and the heterocycloalkyl, heteroarylene, and heteroaryl each include one or more hetero atoms selected from B, N, O, S, P(═O), Si and P.

As described herein, the term ‘(C1-C30)alkyl’ group represents preferably (C1-C20)alkyl and more preferably (C1-C10)alkyl, and the term ‘(C6-C30)aryl’ group represents preferably (C6-C20)aryl and more preferably (C6-C12)aryl. The term ‘(C2-C30)heteroaryl’ group represents preferably (C2-C20)heteroaryl and more preferably (C2-C12)heteroaryl. The term ‘(C3-C30)cycloalkyl’ group represents preferably (C3-C20)cycloalkyl and more preferably (C3-C7)cycloalkyl.

Further, as described herein, the term “substituted” in the description “substituted or unsubstituted”, means to be further substituted with an unsubstituted substituent, and substituents further substituted at L₁, L₂, Ar₁, Ar₂, Z₁, Z₂, R₁ through R₈, R₁₁ through R₁₄, R₂₁ through R₂₇ and R₃₁ through R₃₅ independently represent one or more selected from the group consisting of deuterium, halogen, (C1-C30)alkyl, (C1-C30)alkyl substituted or unsubstituted with halogen, (C6-C30)aryl, (C2-C30)heteroaryl, (C2-C30)heteroaryl substituted or unsubstituted with (C1-C30)alkyl, (C2-C30)heteroaryl substituted or unsubstituted with (C6-C30)aryl, (C3-C30)cycloalkyl, 5- to 7-membered heterocycloalkyl, tri(C6-C30)alkylsilyl, tri(C1-C30)arylsilyl, di(C1-C30)alkyl(C6-C30)arylsilyl, (C1-C30)alkyldi(C6-C30)arylsilyl, (C2-C30)alkenyl, (C2-C30)alkynyl, cyano, carbazolyl, di(C1-C30)alkylamino, di(C6-C30)arylamino, (C1-C30)alkyl(C6-C30)arylamino, di(C6-C30)arylboronyl, di(C1-C30)alkylboronyl, (C1-C30)alkyl(C6-C30)arylboronyl, (C6-C30)ar(C1-C30)alkyl, (C1-C30)alkyl(C6-C30)aryl, carboxyl, nitro, and hydroxy.

Preferably, the L₁, and L₂ independently may selected from the group consisting of a single bond, phenylene, naphthylene, biphenylene, terphenylene, anthrylene, indenylene, fluorenylene, phenanthrylene, triphenylenylene, pyrenylene, perylenylene, chrysenylene, naphthacenylene, fluoranthenylene, phenylene-naphthanylene, furylene, thiophenylene, pyrolylene, imidazolylene, pyrazolylene, thiazolylene, thiadiazolylene, isothiazolylene, isoxazolylene, oxazolylene, oxadiazolylene, triazinylene, tetrazinylene, triazolylene, tetrazolylene, furazanylene, pyridylene, pyrazinylene, pyrimidinylene, pyridazinylene, benzofuranylene, benzothiopenylene, isobenzofuranylene, benzoimidazolylene, benzothiazolylene, benzoisothiazolylene, benzoisoxazolylene, benzooxazolylene, isoindolylene, indolylene, indazolylene, benzothiadiazolylene, quinolylene, isoquinolylene, cinnolinylene, quinazolinylene, quinoxalinylene, carbozolylene, phenanthridinylene, benzodioxolylene, dibenzofuranylene, and dibenzothiophenylene, and the L₁ and L₂ independently may be further substituted with one or more substitutents selected from the group consisting of deuterium, halogen, (C1-C30)alkyl, (C1-C30)alkyl substituted or unsubstituted with halogen, (C6-C30)aryl, (C3-C30)heteroaryl, (C3-C30)heteroaryl substituted or unsubstituted with (C6-C30)aryl, (C3-C30)cycloalkyl, N-carbazolyl, (C6-C30)ar(C1-C30)alkyl, and (C1-C30)alkyl(C6-C30)aryl.

Specifically, the

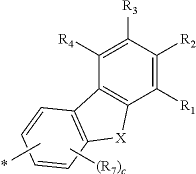

is selected from the structure below, but not limited thereto.

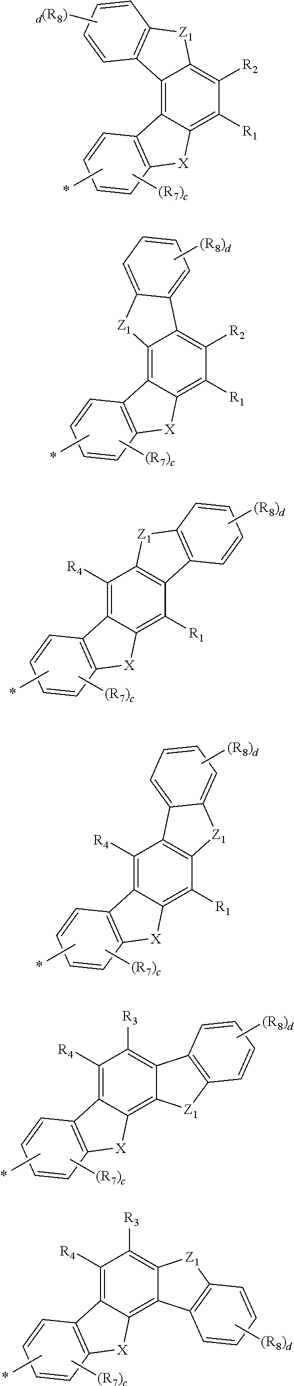

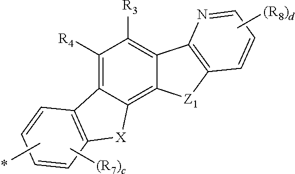

Wherein, X represents —O—, —S—, —CR₁₁R₁₂— or N-L₁-Ar₁; Z₁ represents —O—, —S—, —CR₃₁R₃₂—, —SiR₃₃R₃₄— or —NR₃₅—, R₁ through R₄ independently represent hydrogen, deuterium, halogen, substituted or unsubstituted (C1-C30)alkyl, substituted or unsubstituted (C6-C30)aryl, substituted or unsubstituted (C3-C30)heteroaryl, substituted or unsubstituted (C3-C30)cycloalkyl, substituted or unsubstituted 5- to 7-membered heterocycloalkyl, substituted or unsubstituted (C6-C30)ar(C1-C30)alkyl, substituted or unsubstituted (C6-C30)aryl fused with one or more (C3-C30)cycloalkyl, 5- to 7-membered heterocycloalkyl fused with one or more substituted or unsubstituted aromatic rings, (C3-C30)cycloalkyl fused with one or more substituted or unsubstituted aromatic rings, —NR₂₁R₂₂, —SiR₂₃R₂₄R₂₅, —SR₂₆, —OR₂₇, (C2-C30)alkenyl, (C2-C30)alkynyl, cyano, nitro, or hydroxy.

More specifically,

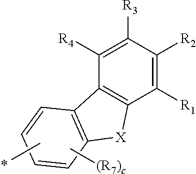

represents

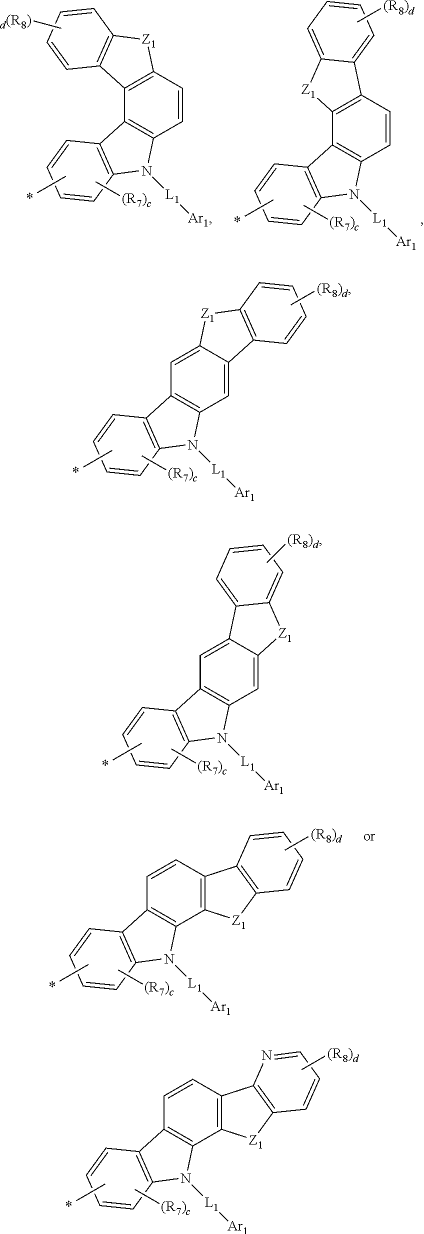

Y represents —O—, —S—, —CR₁₃R₁₄— or N-L₂-Ar₂; Z₁ represents —O—, —S—, —CR₃₁R₃₂—, —SiR₃₃R₃₄— or —NR₃₅—, L₁ and L₂ independently represent a single bond, (C6-C30)arylene, or (C3-C30)heteroarylene; Ar₁ and Ar₂ independently represent hydrogen, deuterium, halogen, (C1-C30)alkyl, (C6-C30)aryl, or (C3-C30)heteroaryl; R₅ through R₈ independently represent hydrogen, deuterium, halogen, (C1-C30)alkyl, (C6-C30)aryl, or (C3-C30)heteroaryl; R₁₃, R₁₄, R₃₁ through R₃₅ independently represent hydrogen, deuterium, (C1-C30)alkyl, (C6-C30)aryl, or (C3-C30)heteroaryl; the arylene and heteroarylene of L₁ and L₂, the alkyl, aryl, or heteroaryl of R₅ through R₈, the alkyl, aryl, or heteroaryl of Ar₁ and Ar₂, R₁₃, R₁₄, R₃₁ through R₃₅ independently may be further substituted from one or more selected from the group consisting of deuterium, halogen, (C1-C30)alkyl, (C1-C30)alkyl substituted with halogen, (C6-C30)aryl, (C3-C30)heteroaryl, (C3-C30)heteroaryl substituted with (C6-C30)aryl, (C3-C30)cycloalkyl, N-carbazolyl, (C6-C30)ar(C1-C30)alkyl, and (C1-C30)alkyl(C6-C30)aryl.

The compounds below may be employed as the compound for an organic electronic material according to the present invention by way of representative examples.

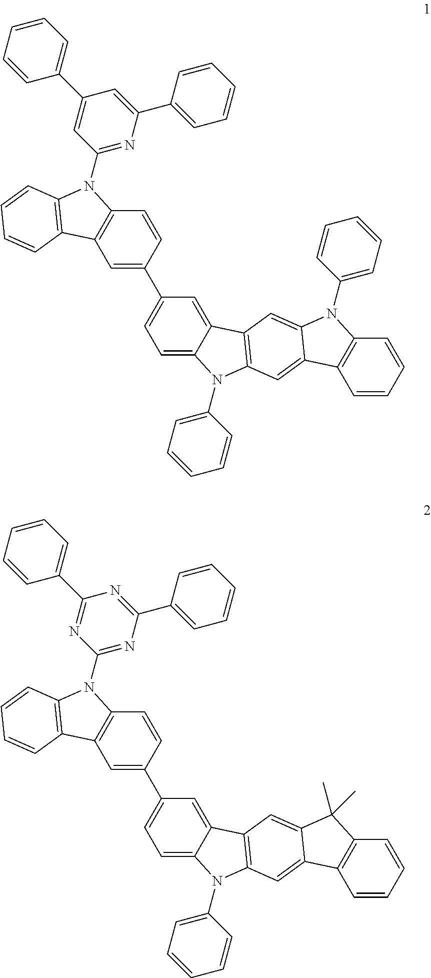 [SMILES: c1ccc(-c2cc(-c3ccccc3)nc(-n3c4ccccc4c4cc(-c5ccc6c(c5)c5cc7c(cc5n6-c5ccccc5)c5ccccc5n7-c5ccccc5)ccc43)c2)cc1 ; CC1(C)c2ccccc2-c2cc3c(cc21)c1cc(-c2ccc4c(c2)c2ccccc2n4-c2nc(-c4ccccc4)nc(-c4ccccc4)n2)ccc1n3-c1ccccc1]

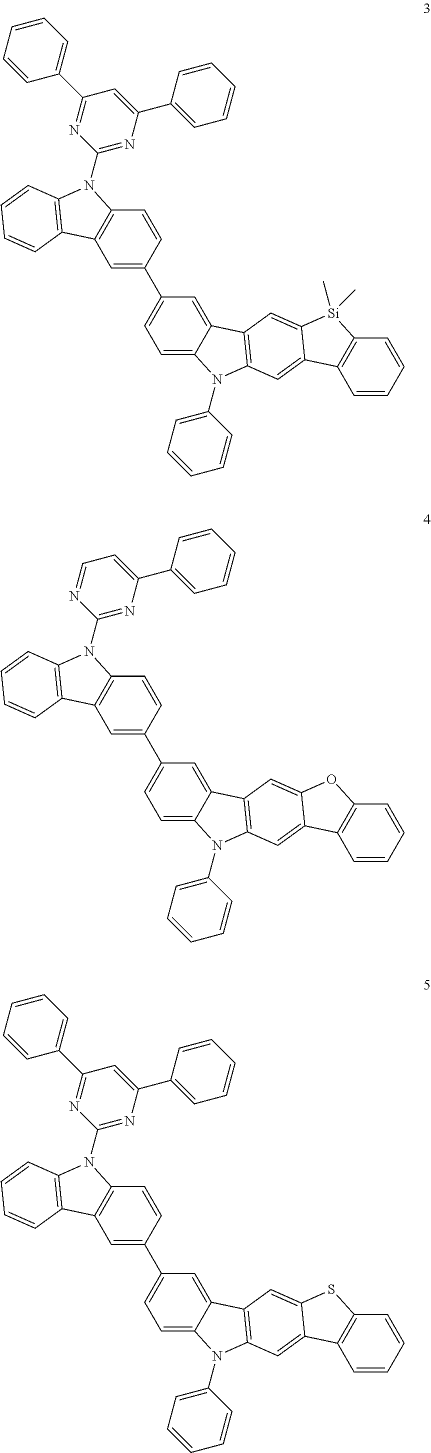 [SMILES: C[Si]1(C)c2ccccc2-c2cc3c(cc21)c1cc(-c2ccc4c(c2)c2ccccc2n4-c2nc(-c4ccccc4)cc(-c4ccccc4)n2)ccc1n3-c1ccccc1 ; c1ccc(-c2ccnc(-n3c4ccccc4c4cc(-c5ccc6c(c5)c5cc7oc8ccccc8c7cc5n6-c5ccccc5)ccc43)n2)cc1 ; c1ccc(-c2cc(-c3ccccc3)nc(-n3c4ccccc4c4cc(-c5ccc6c(c5)c5cc7sc8ccccc8c7cc5n6-c5ccccc5)ccc43)n2)cc1]

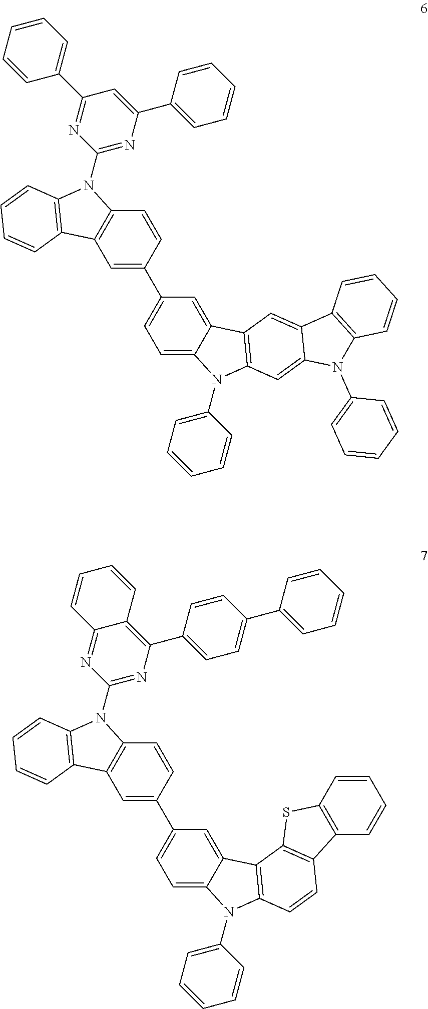 [SMILES: c1ccc(-c2cc(-c3ccccc3)nc(-n3c4ccccc4c4cc(-c5ccc6c(c5)c5cc7c8ccccc8n(-c8ccccc8)c7cc5n6-c5ccccc5)ccc43)n2)cc1 ; c1ccc(-c2ccc(-c3nc(-n4c5ccccc5c5cc(-c6ccc7c(c6)c6c8sc9ccccc9c8ccc6n7-c6ccccc6)ccc54)nc4ccccc34)cc2)cc1]

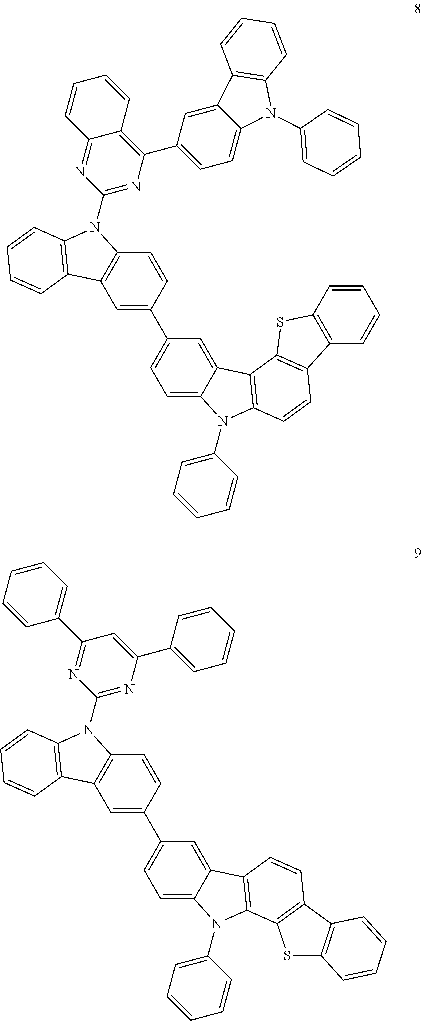 [SMILES: c1ccc(-n2c3ccccc3c3cc(-c4nc(-n5c6ccccc6c6cc(-c7ccc8c(c7)c7c9sc%10ccccc%10c9ccc7n8-c7ccccc7)ccc65)nc5ccccc45)ccc32)cc1 ; c1ccc(-c2cc(-c3ccccc3)nc(-n3c4ccccc4c4cc(-c5ccc6c(c5)c5ccc7c8ccccc8sc7c5n6-c5ccccc5)ccc43)n2)cc1]

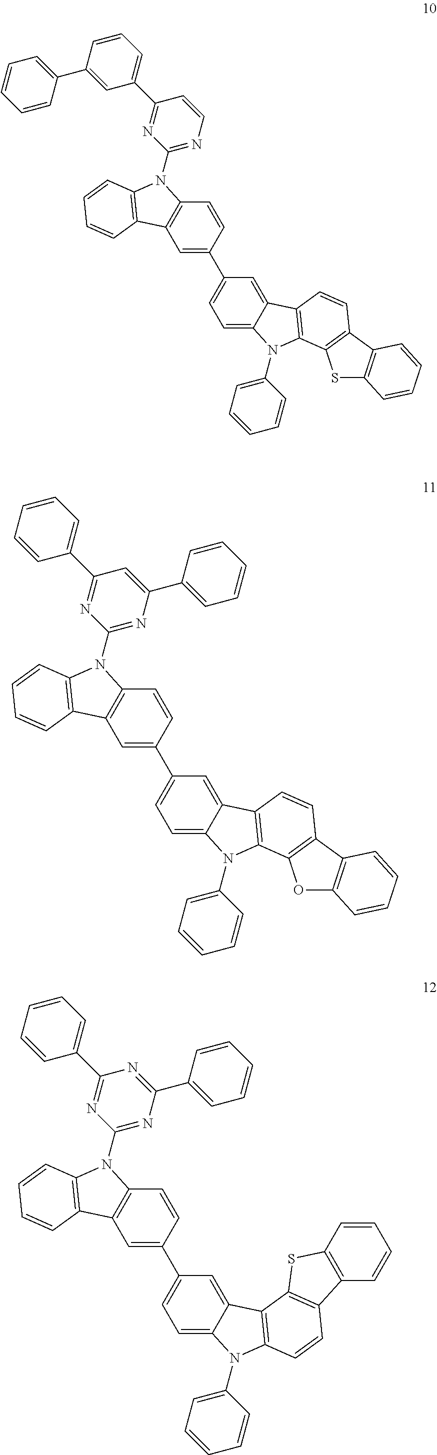 [SMILES: c1ccc(-c2cccc(-c3ccnc(-n4c5ccccc5c5cc(-c6ccc7c(c6)c6ccc8c9ccccc9sc8c6n7-c6ccccc6)ccc54)n3)c2)cc1 ; c1ccc(-c2cc(-c3ccccc3)nc(-n3c4ccccc4c4cc(-c5ccc6c(c5)c5ccc7c8ccccc8oc7c5n6-c5ccccc5)ccc43)n2)cc1 ; c1ccc(-c2nc(-c3ccccc3)nc(-n3c4ccccc4c4cc(-c5ccc6c(c5)c5c7sc8ccccc8c7ccc5n6-c5ccccc5)ccc43)n2)cc1]

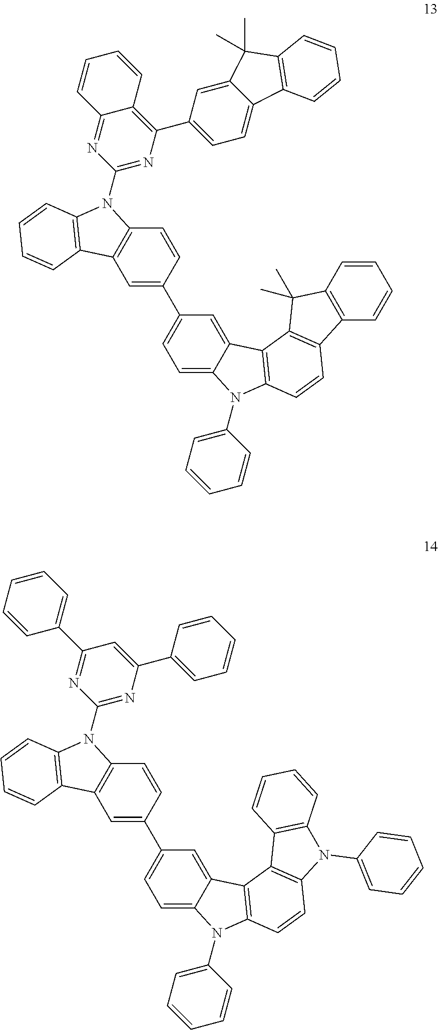 [SMILES: CC1(C)c2ccccc2-c2ccc(-c3nc(-n4c5ccccc5c5cc(-c6ccc7c(c6)c6c8c(ccc6n7-c6ccccc6)-c6ccccc6C8(C)C)ccc54)nc4ccccc34)cc21 ; c1ccc(-c2cc(-c3ccccc3)nc(-n3c4ccccc4c4cc(-c5ccc6c(c5)c5c7c8ccccc8n(-c8ccccc8)c7ccc5n6-c5ccccc5)ccc43)n2)cc1]

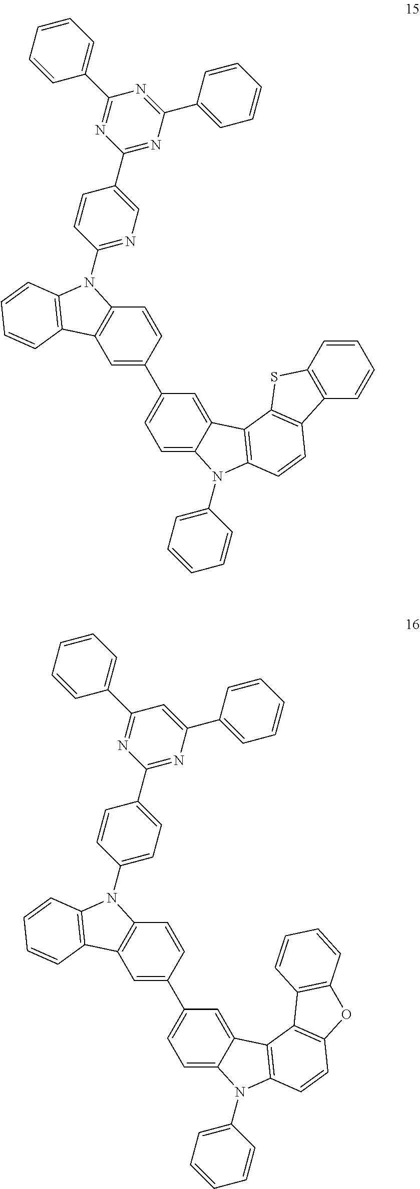 [SMILES: c1ccc(-c2nc(-c3ccccc3)nc(-c3ccc(-n4c5ccccc5c5cc(-c6ccc7c(c6)c6c8sc9ccccc9c8ccc6n7-c6ccccc6)ccc54)nc3)n2)cc1 ; c1ccc(-c2cc(-c3ccccc3)nc(-c3ccc(-n4c5ccccc5c5cc(-c6ccc7c(c6)c6c8c(ccc6n7-c6ccccc6)oc6ccccc68)ccc54)cc3)n2)cc1]

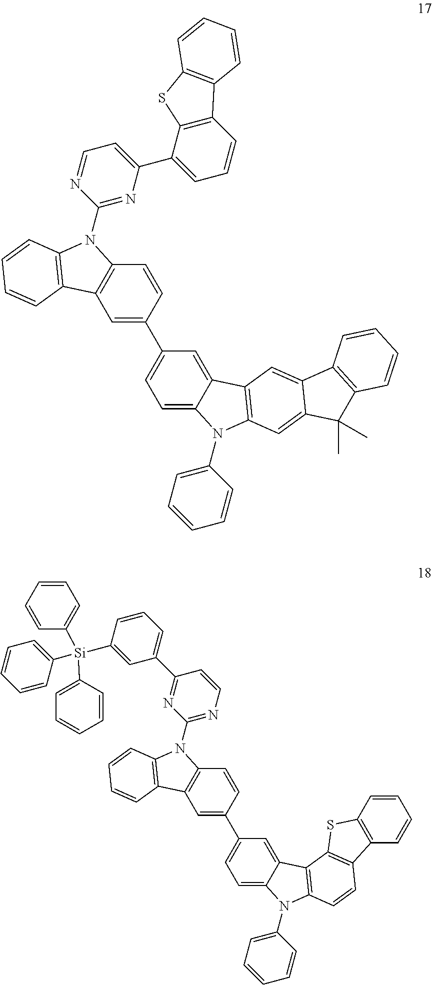 [SMILES: CC1(C)c2ccccc2-c2cc3c4cc(-c5ccc6c(c5)c5ccccc5n6-c5nccc(-c6cccc7c6sc6ccccc67)n5)ccc4n(-c4ccccc4)c3cc21 ; c1ccc(-n2c3ccc(-c4ccc5c(c4)c4ccccc4n5-c4nccc(-c5cccc([Si](c6ccccc6)(c6ccccc6)c6ccccc6)c5)n4)cc3c3c4sc5ccccc5c4ccc32)cc1]

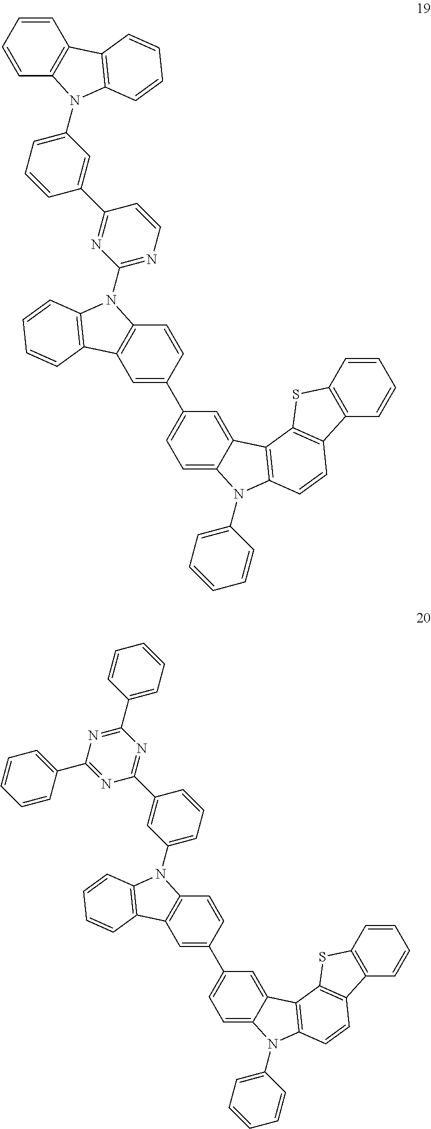 [SMILES: c1ccc(-n2c3ccc(-c4ccc5c(c4)c4ccccc4n5-c4nccc(-c5cccc(-n6c7ccccc7c7ccccc76)c5)n4)cc3c3c4sc5ccccc5c4ccc32)cc1 ; c1ccc(-c2nc(-c3ccccc3)nc(-c3cccc(-n4c5ccccc5c5cc(-c6ccc7c(c6)c6c8sc9ccccc9c8ccc6n7-c6ccccc6)ccc54)c3)n2)cc1]

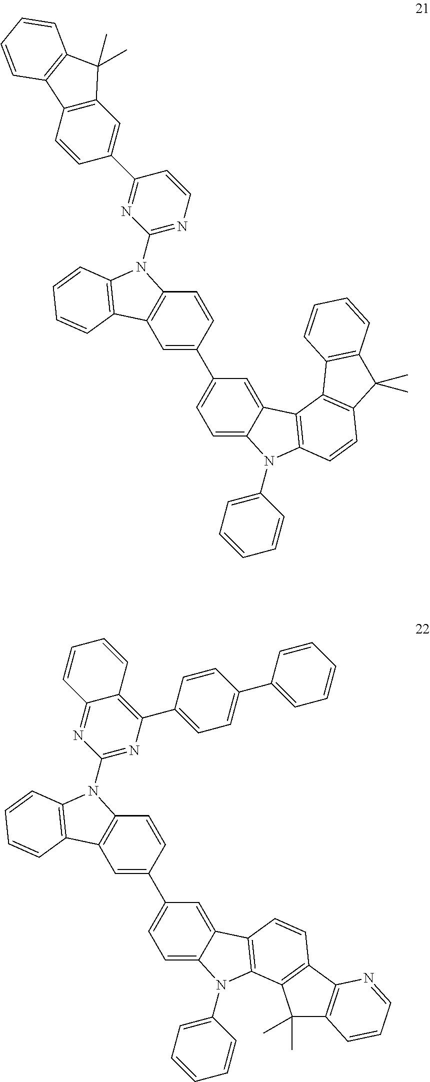 [SMILES: CC1(C)c2ccccc2-c2ccc(-c3ccnc(-n4c5ccccc5c5cc(-c6ccc7c(c6)c6c8c(ccc6n7-c6ccccc6)C(C)(C)c6ccccc6-8)ccc54)n3)cc21 ; CC1(C)c2cccnc2-c2ccc3c4cc(-c5ccc6c(c5)c5ccccc5n6-c5nc(-c6ccc(-c7ccccc7)cc6)c6ccccc6n5)ccc4n(-c4ccccc4)c3c21]

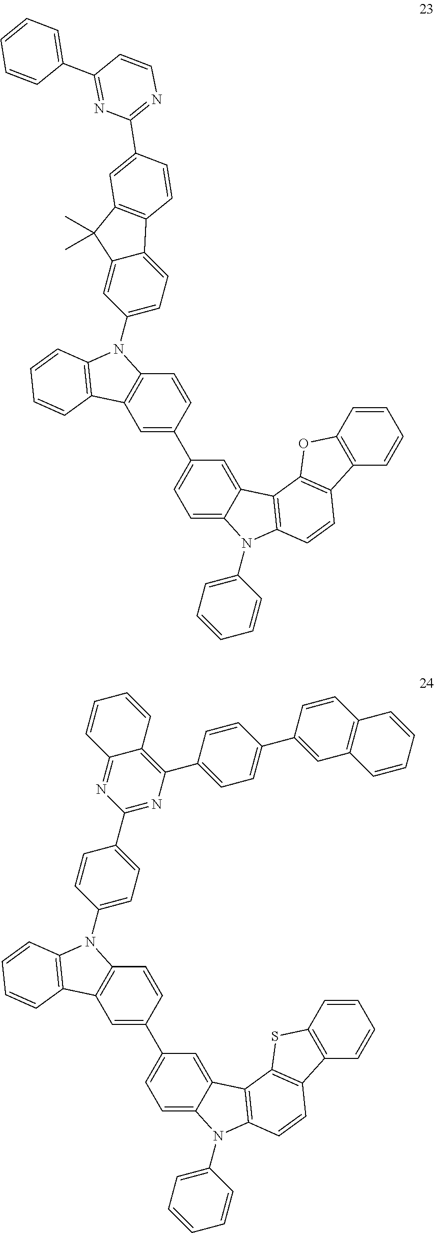 [SMILES: CC1(C)c2cc(-c3nccc(-c4ccccc4)n3)ccc2-c2ccc(-n3c4ccccc4c4cc(-c5ccc6c(c5)c5c7oc8ccccc8c7ccc5n6-c5ccccc5)ccc43)cc21 ; c1ccc(-n2c3ccc(-c4ccc5c(c4)c4ccccc4n5-c4ccc(-c5nc(-c6ccc(-c7ccc8ccccc8c7)cc6)c6ccccc6n5)cc4)cc3c3c4sc5ccccc5c4ccc32)cc1]

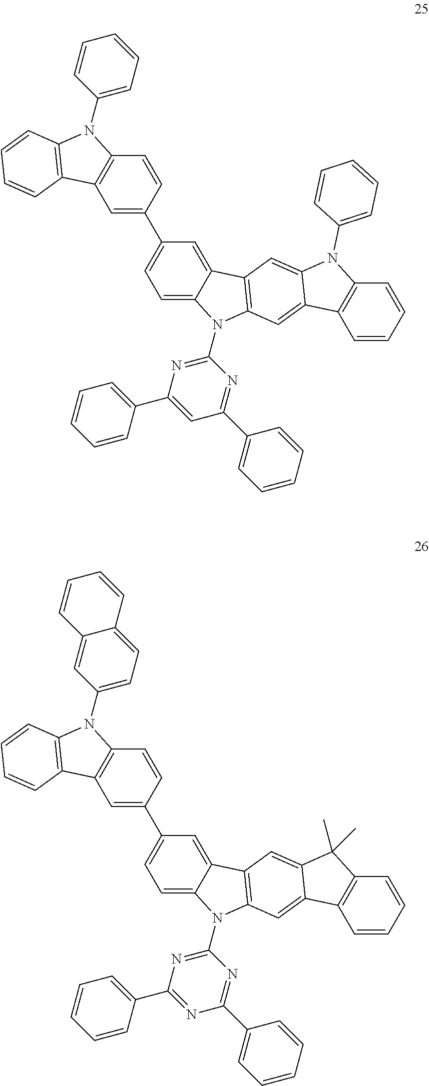 [SMILES: c1ccc(-c2cc(-c3ccccc3)nc(-n3c4ccc(-c5ccc6c(c5)c5ccccc5n6-c5ccccc5)cc4c4cc5c(cc43)c3ccccc3n5-c3ccccc3)n2)cc1 ; CC1(C)c2ccccc2-c2cc3c(cc21)c1cc(-c2ccc4c(c2)c2ccccc2n4-c2ccc4ccccc4c2)ccc1n3-c1nc(-c2ccccc2)nc(-c2ccccc2)n1]

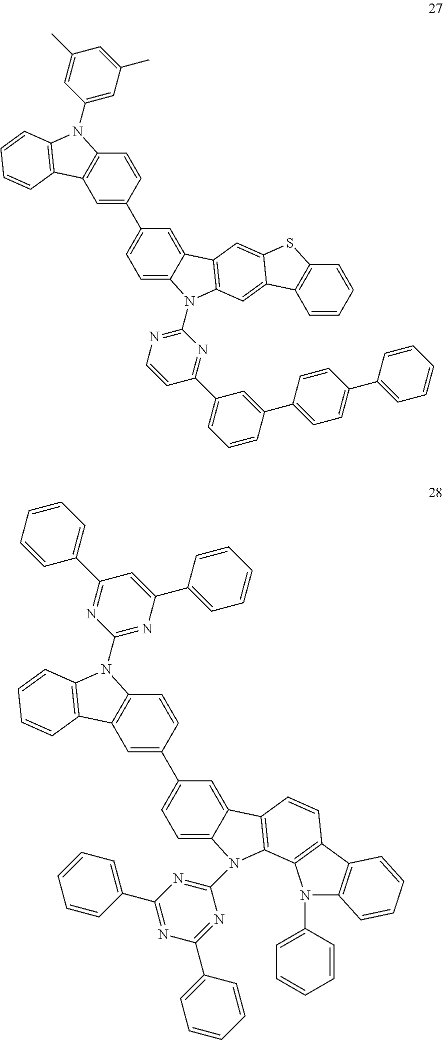 [SMILES: Cc1cc(C)cc(-n2c3ccccc3c3cc(-c4ccc5c(c4)c4cc6sc7ccccc7c6cc4n5-c4nccc(-c5cccc(-c6ccc(-c7ccccc7)cc6)c5)n4)ccc32)c1 ; c1ccc(-c2cc(-c3ccccc3)nc(-n3c4ccccc4c4cc(-c5ccc6c(c5)c5ccc7c8ccccc8n(-c8ccccc8)c7c5n6-c5nc(-c6ccccc6)nc(-c6ccccc6)n5)ccc43)n2)cc1]

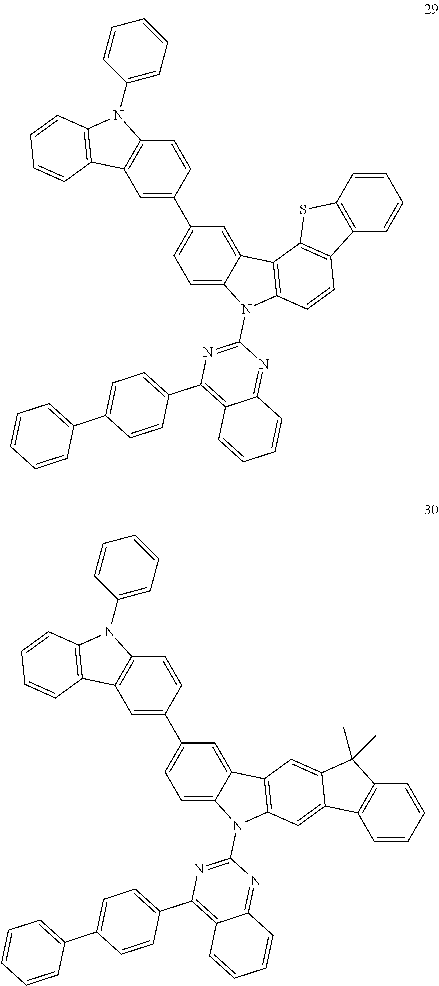 [SMILES: c1ccc(-c2ccc(-c3nc(-n4c5ccc(-c6ccc7c(c6)c6ccccc6n7-c6ccccc6)cc5c5c6sc7ccccc7c6ccc54)nc4ccccc34)cc2)cc1 ; CC1(C)c2ccccc2-c2cc3c(cc21)c1cc(-c2ccc4c(c2)c2ccccc2n4-c2ccccc2)ccc1n3-c1nc(-c2ccc(-c3ccccc3)cc2)c2ccccc2n1]

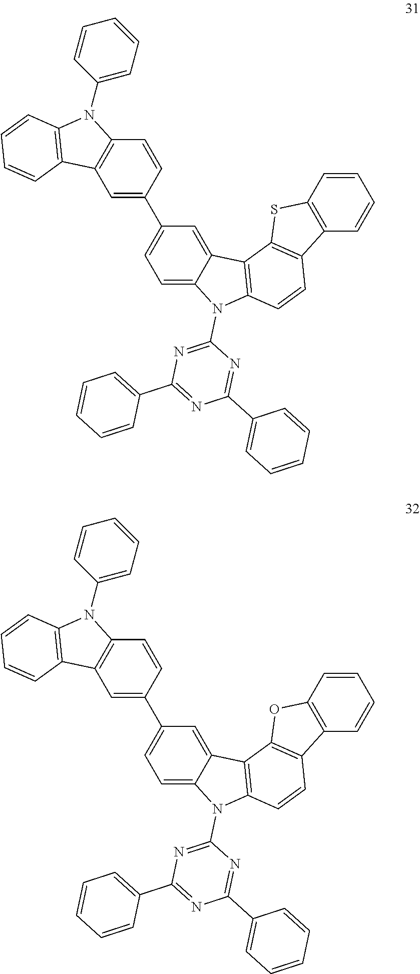 [SMILES: c1ccc(-c2nc(-c3ccccc3)nc(-n3c4ccc(-c5ccc6c(c5)c5ccccc5n6-c5ccccc5)cc4c4c5sc6ccccc6c5ccc43)n2)cc1 ; c1ccc(-c2nc(-c3ccccc3)nc(-n3c4ccc(-c5ccc6c(c5)c5ccccc5n6-c5ccccc5)cc4c4c5oc6ccccc6c5ccc43)n2)cc1]

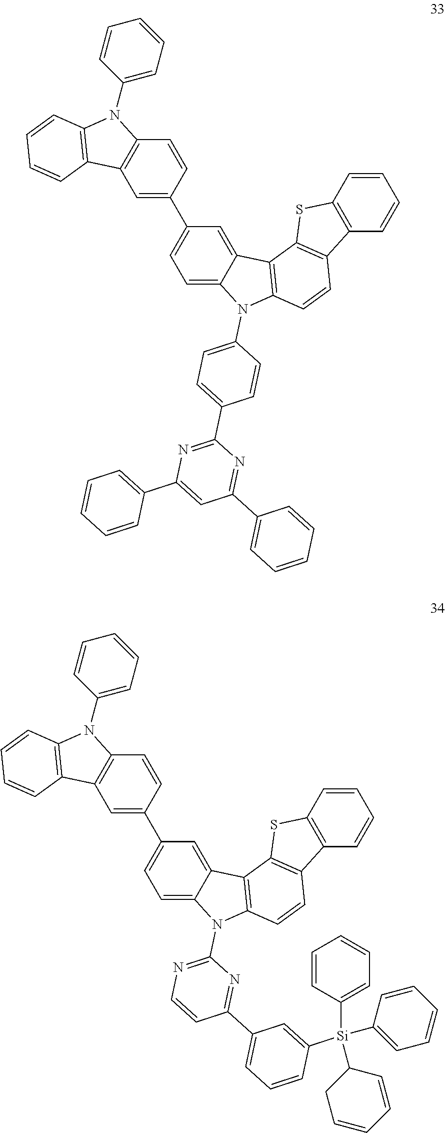 [SMILES: c1ccc(-c2cc(-c3ccccc3)nc(-c3ccc(-n4c5ccc(-c6ccc7c(c6)c6ccccc6n7-c6ccccc6)cc5c5c6sc7ccccc7c6ccc54)cc3)n2)cc1 ; C1=CCC([Si](c2ccccc2)(c2ccccc2)c2cccc(-c3ccnc(-n4c5ccc(-c6ccc7c(c6)c6ccccc6n7-c6ccccc6)cc5c5c6sc7ccccc7c6ccc54)n3)c2)C=C1]

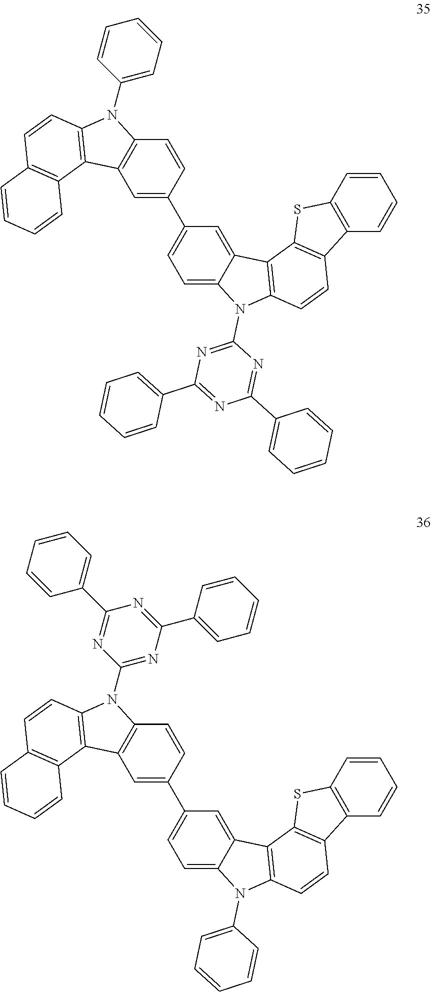 [SMILES: c1ccc(-c2nc(-c3ccccc3)nc(-n3c4ccc(-c5ccc6c(c5)c5c7ccccc7ccc5n6-c5ccccc5)cc4c4c5sc6ccccc6c5ccc43)n2)cc1 ; c1ccc(-c2nc(-c3ccccc3)nc(-n3c4ccc(-c5ccc6c(c5)c5c7sc8ccccc8c7ccc5n6-c5ccccc5)cc4c4c5ccccc5ccc43)n2)cc1]

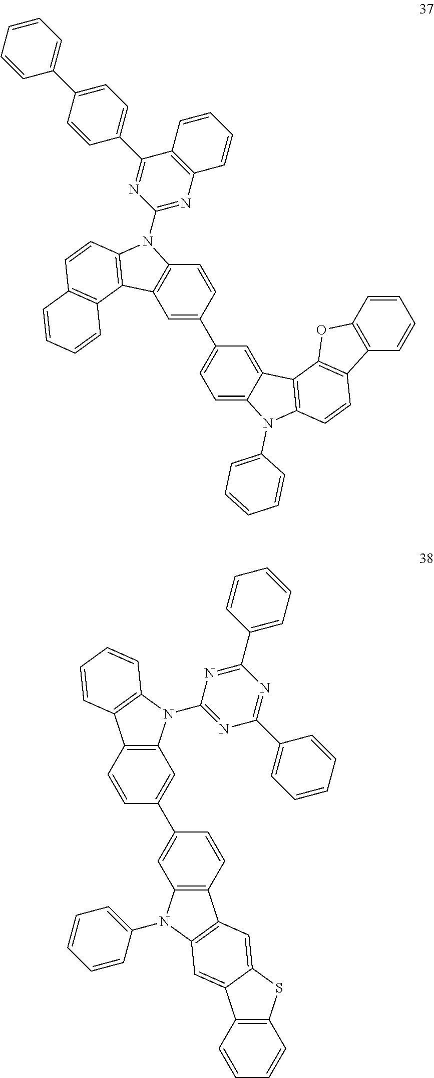 [SMILES: c1ccc(-c2ccc(-c3nc(-n4c5ccc(-c6ccc7c(c6)c6c8oc9ccccc9c8ccc6n7-c6ccccc6)cc5c5c6ccccc6ccc54)nc4ccccc34)cc2)cc1 ; c1ccc(-c2nc(-c3ccccc3)nc(-n3c4ccccc4c4ccc(-c5ccc6c7cc8sc9ccccc9c8cc7n(-c7ccccc7)c6c5)cc43)n2)cc1]

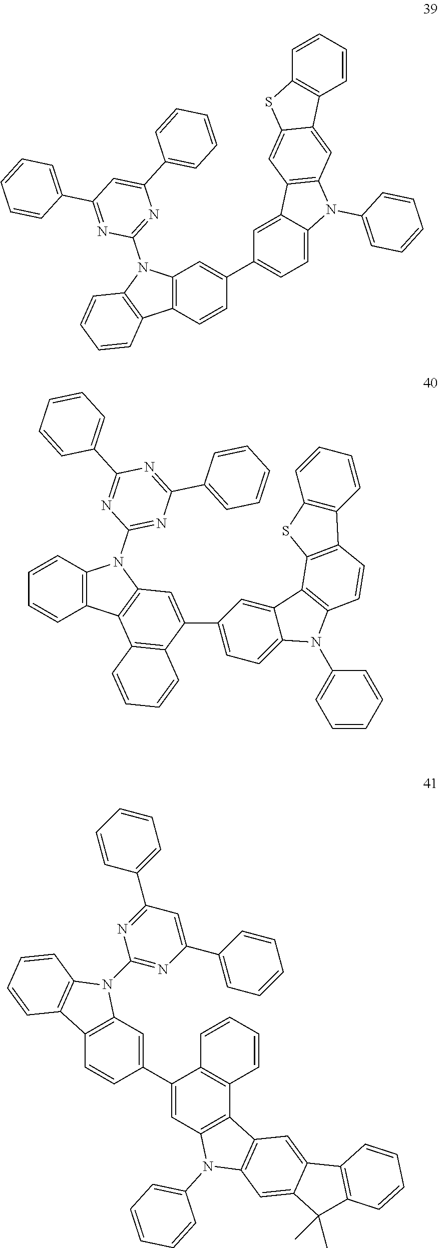

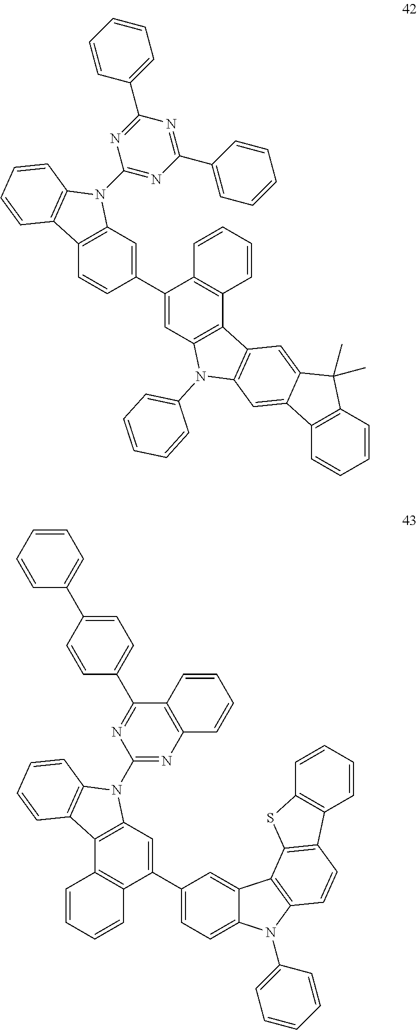 [SMILES: CC1(C)c2ccccc2-c2cc3c(cc21)c1c2ccccc2c(-c2ccc4c5ccccc5n(-c5nc(-c6ccccc6)nc(-c6ccccc6)n5)c4c2)cc1n3-c1ccccc1 ; c1ccc(-c2ccc(-c3nc(-n4c5ccccc5c5c6ccccc6c(-c6ccc7c(c6)c6c8sc9ccccc9c8ccc6n7-c6ccccc6)cc54)nc4ccccc34)cc2)cc1]

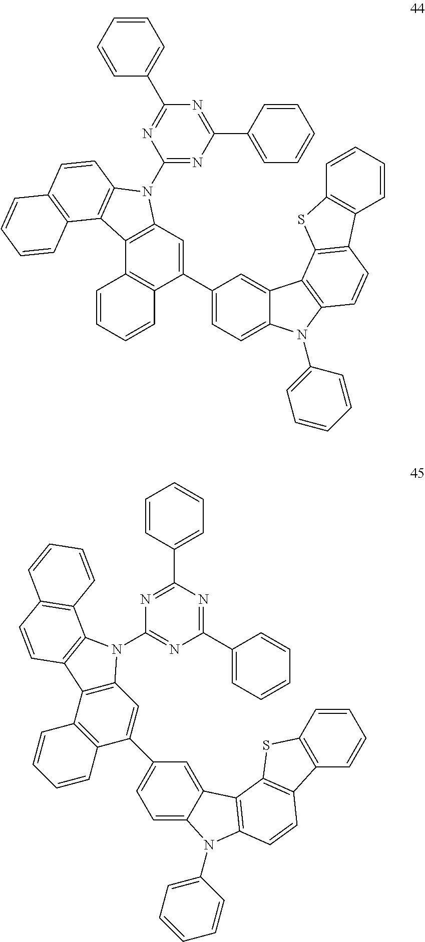 [SMILES: c1ccc(-c2nc(-c3ccccc3)nc(-n3c4ccc5ccccc5c4c4c5ccccc5c(-c5ccc6c(c5)c5c7sc8ccccc8c7ccc5n6-c5ccccc5)cc43)n2)cc1 ; c1ccc(-c2nc(-c3ccccc3)nc(-n3c4cc(-c5ccc6c(c5)c5c7sc8ccccc8c7ccc5n6-c5ccccc5)c5ccccc5c4c4ccc5ccccc5c43)n2)cc1]

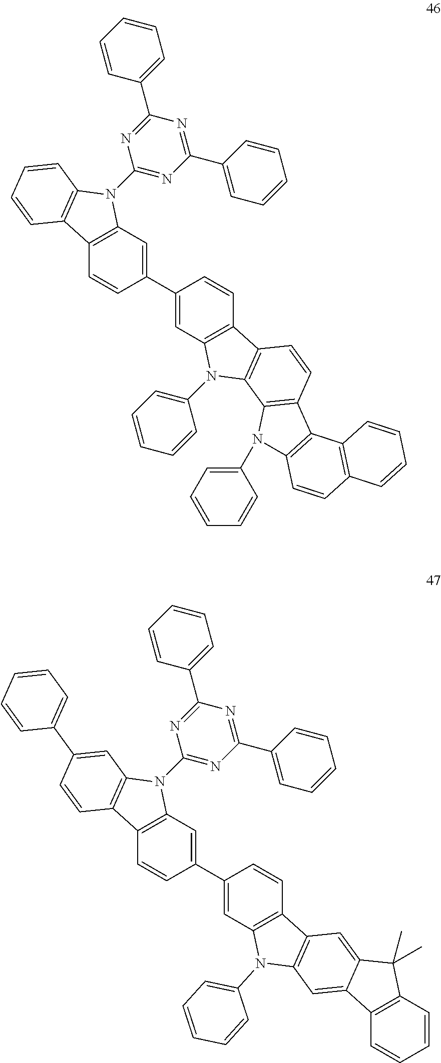 [SMILES: c1ccc(-c2nc(-c3ccccc3)nc(-n3c4ccccc4c4ccc(-c5ccc6c7ccc8c9c%10ccccc%10ccc9n(-c9ccccc9)c8c7n(-c7ccccc7)c6c5)cc43)n2)cc1 ; CC1(C)c2ccccc2-c2cc3c(cc21)c1ccc(-c2ccc4c5ccc(-c6ccccc6)cc5n(-c5nc(-c6ccccc6)nc(-c6ccccc6)n5)c4c2)cc1n3-c1ccccc1]

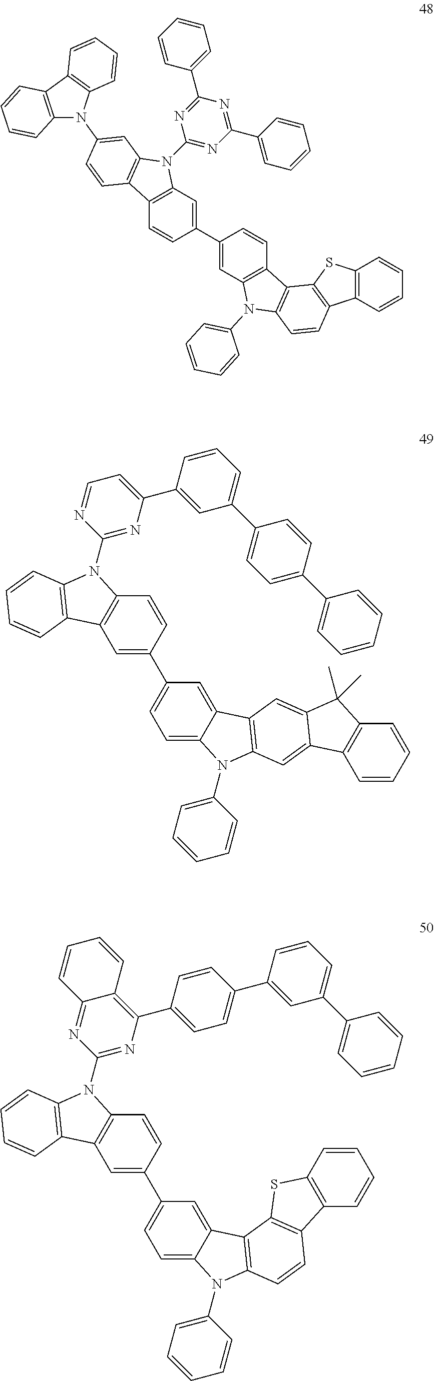 [SMILES: c1ccc(-c2nc(-c3ccccc3)nc(-n3c4cc(-c5ccc6c7c8sc9ccccc9c8ccc7n(-c7ccccc7)c6c5)ccc4c4ccc(-n5c6ccccc6c6ccccc65)cc43)n2)cc1 ; CC1(C)c2ccccc2-c2cc3c(cc21)c1cc(-c2ccc4c(c2)c2ccccc2n4-c2nccc(-c4cccc(-c5ccc(-c6ccccc6)cc5)c4)n2)ccc1n3-c1ccccc1 ; c1ccc(-c2cccc(-c3ccc(-c4nc(-n5c6ccccc6c6cc(-c7ccc8c(c7)c7c9sc%10ccccc%10c9ccc7n8-c7ccccc7)ccc65)nc5ccccc45)cc3)c2)cc1]

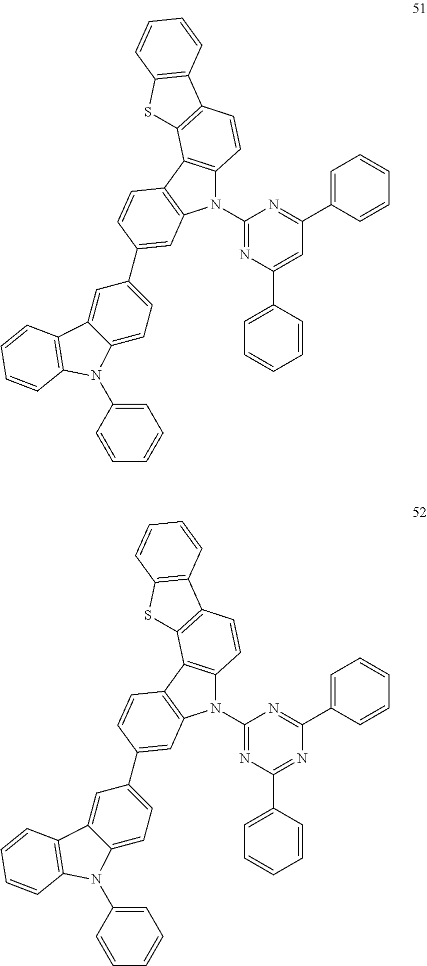 [SMILES: c1ccc(-c2cc(-c3ccccc3)nc(-n3c4cc(-c5ccc6c(c5)c5ccccc5n6-c5ccccc5)ccc4c4c5sc6ccccc6c5ccc43)n2)cc1 ; c1ccc(-c2nc(-c3ccccc3)nc(-n3c4cc(-c5ccc6c(c5)c5ccccc5n6-c5ccccc5)ccc4c4c5sc6ccccc6c5ccc43)n2)cc1]

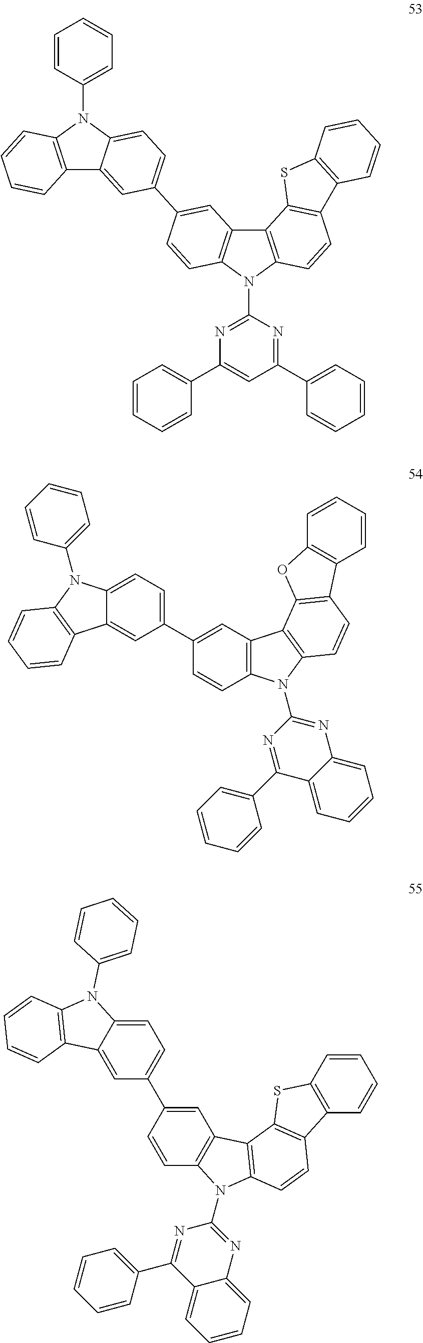 [SMILES: c1ccc(-c2cc(-c3ccccc3)nc(-n3c4ccc(-c5ccc6c(c5)c5ccccc5n6-c5ccccc5)cc4c4c5sc6ccccc6c5ccc43)n2)cc1 ; c1ccc(-c2nc(-n3c4ccc(-c5ccc6c(c5)c5ccccc5n6-c5ccccc5)cc4c4c5oc6ccccc6c5ccc43)nc3ccccc23)cc1 ; c1ccc(-c2nc(-n3c4ccc(-c5ccc6c(c5)c5ccccc5n6-c5ccccc5)cc4c4c5sc6ccccc6c5ccc43)nc3ccccc23)cc1]

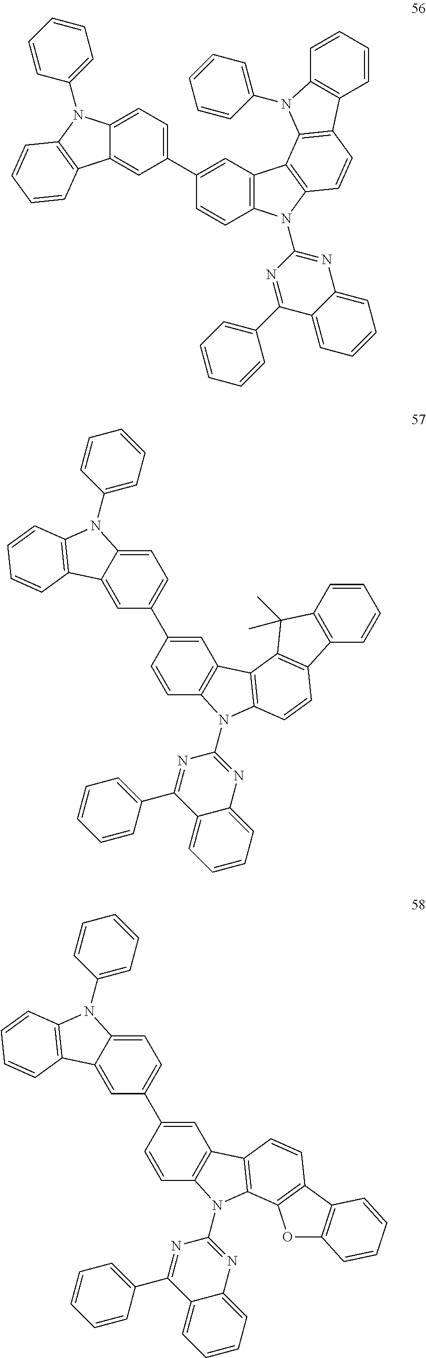 [SMILES: c1ccc(-c2nc(-n3c4ccc(-c5ccc6c(c5)c5ccccc5n6-c5ccccc5)cc4c4c3ccc3c5ccccc5n(-c5ccccc5)c34)nc3ccccc23)cc1 ; CC1(C)c2ccccc2-c2ccc3c(c21)c1cc(-c2ccc4c(c2)c2ccccc2n4-c2ccccc2)ccc1n3-c1nc(-c2ccccc2)c2ccccc2n1 ; c1ccc(-c2nc(-n3c4ccc(-c5ccc6c(c5)c5ccccc5n6-c5ccccc5)cc4c4ccc5c6ccccc6oc5c43)nc3ccccc23)cc1]

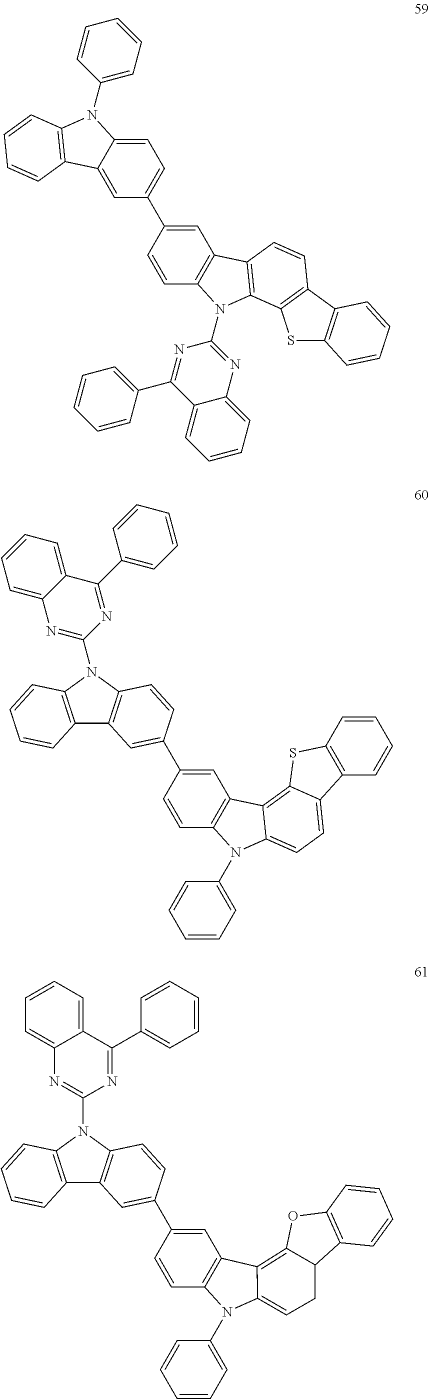

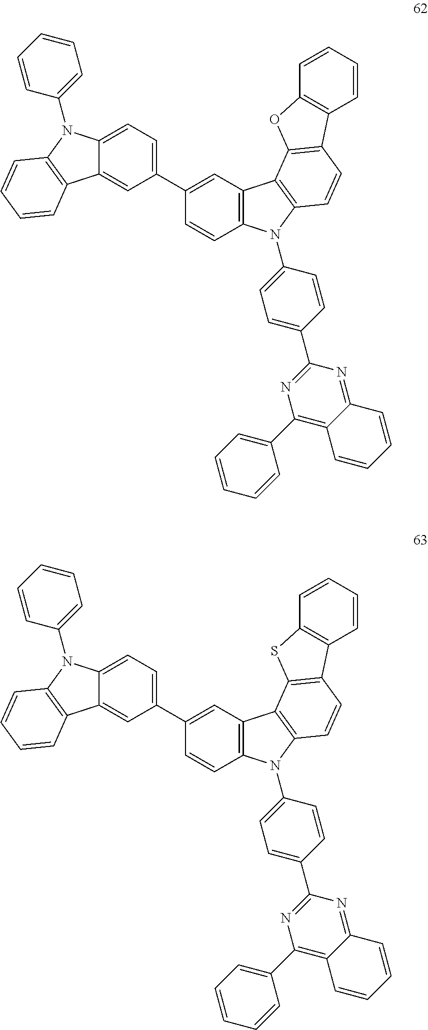 [SMILES: c1ccc(-c2nc(-c3ccc(-n4c5ccc(-c6ccc7c(c6)c6ccccc6n7-c6ccccc6)cc5c5c6oc7ccccc7c6ccc54)cc3)nc3ccccc23)cc1 ; c1ccc(-c2nc(-c3ccc(-n4c5ccc(-c6ccc7c(c6)c6ccccc6n7-c6ccccc6)cc5c5c6sc7ccccc7c6ccc54)cc3)nc3ccccc23)cc1]

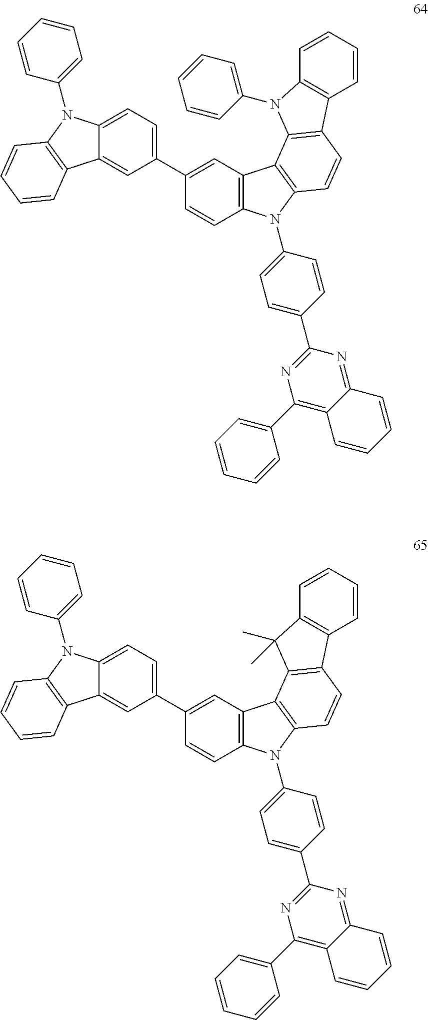 [SMILES: c1ccc(-c2nc(-c3ccc(-n4c5ccc(-c6ccc7c(c6)c6ccccc6n7-c6ccccc6)cc5c5c4ccc4c6ccccc6n(-c6ccccc6)c45)cc3)nc3ccccc23)cc1 ; CC1(C)c2ccccc2-c2ccc3c(c21)c1cc(-c2ccc4c(c2)c2ccccc2n4-c2ccccc2)ccc1n3-c1ccc(-c2nc(-c3ccccc3)c3ccccc3n2)cc1]

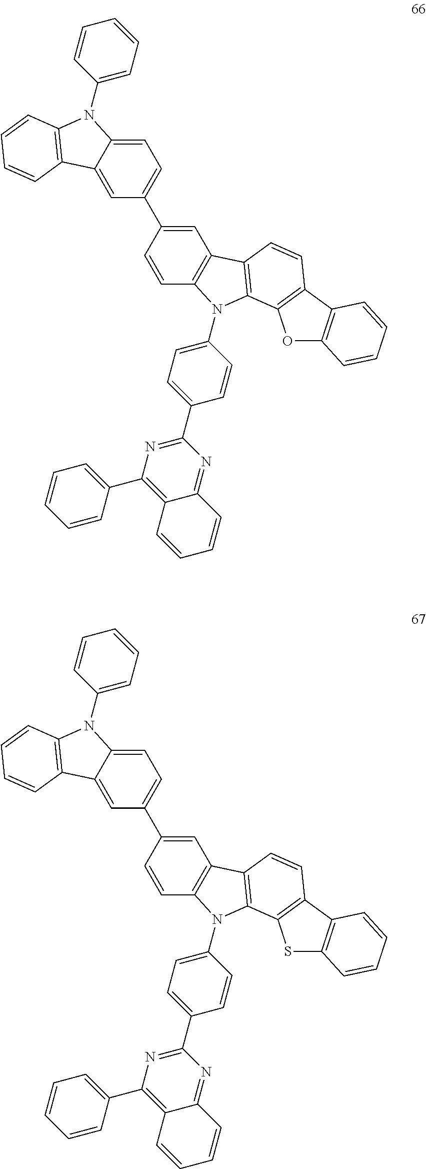 [SMILES: c1ccc(-c2nc(-c3ccc(-n4c5ccc(-c6ccc7c(c6)c6ccccc6n7-c6ccccc6)cc5c5ccc6c7ccccc7oc6c54)cc3)nc3ccccc23)cc1 ; c1ccc(-c2nc(-c3ccc(-n4c5ccc(-c6ccc7c(c6)c6ccccc6n7-c6ccccc6)cc5c5ccc6c7ccccc7sc6c54)cc3)nc3ccccc23)cc1]

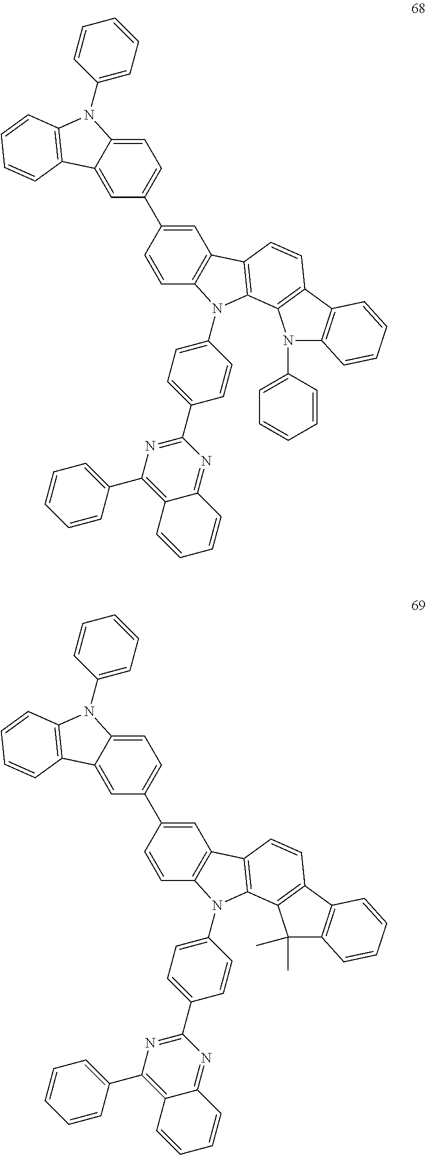 [SMILES: c1ccc(-c2nc(-c3ccc(-n4c5ccc(-c6ccc7c(c6)c6ccccc6n7-c6ccccc6)cc5c5ccc6c7ccccc7n(-c7ccccc7)c6c54)cc3)nc3ccccc23)cc1 ; CC1(C)c2ccccc2-c2ccc3c4cc(-c5ccc6c(c5)c5ccccc5n6-c5ccccc5)ccc4n(-c4ccc(-c5nc(-c6ccccc6)c6ccccc6n5)cc4)c3c21]

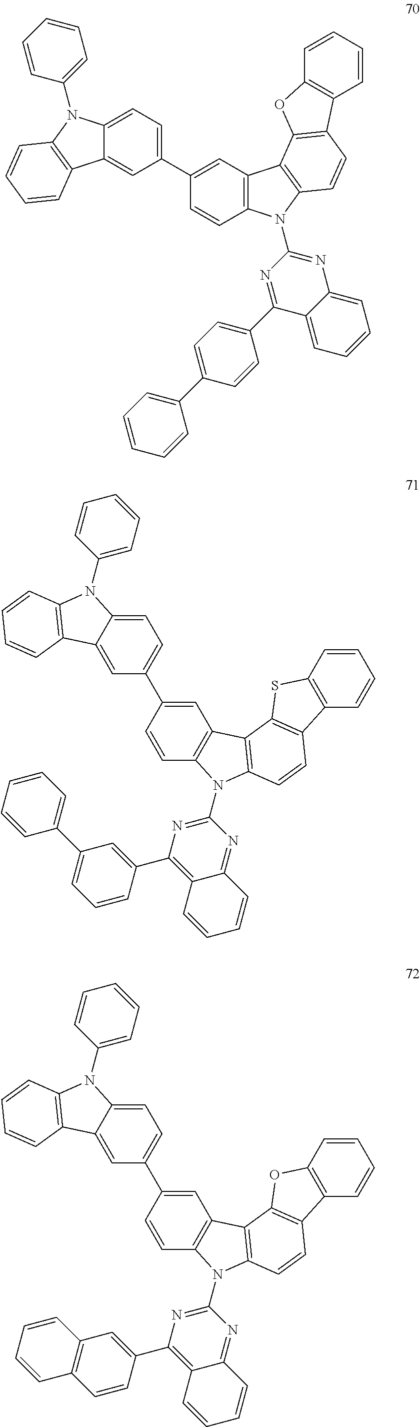 [SMILES: c1ccc(-c2ccc(-c3nc(-n4c5ccc(-c6ccc7c(c6)c6ccccc6n7-c6ccccc6)cc5c5c6oc7ccccc7c6ccc54)nc4ccccc34)cc2)cc1 ; c1ccc(-c2cccc(-c3nc(-n4c5ccc(-c6ccc7c(c6)c6ccccc6n7-c6ccccc6)cc5c5c6sc7ccccc7c6ccc54)nc4ccccc34)c2)cc1 ; c1ccc(-n2c3ccccc3c3cc(-c4ccc5c(c4)c4c6oc7ccccc7c6ccc4n5-c4nc(-c5ccc6ccccc6c5)c5ccccc5n4)ccc32)cc1]

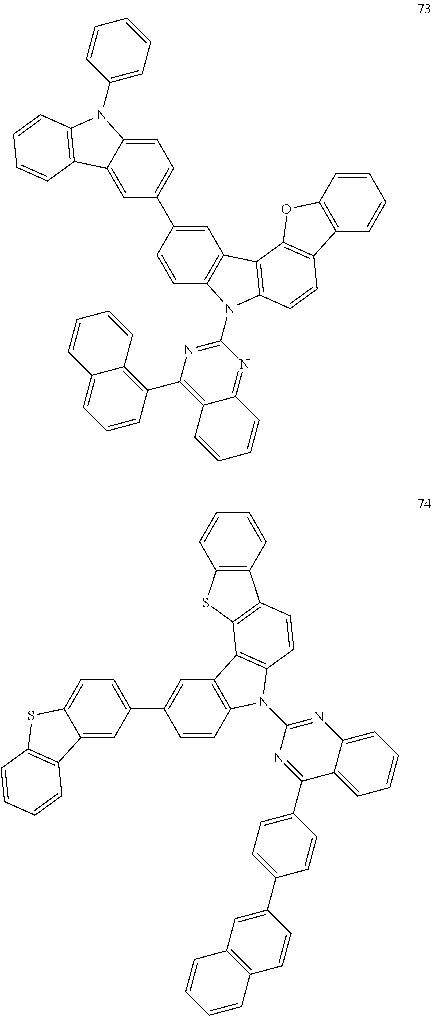 [SMILES: c1ccc(-n2c3ccccc3c3cc(-c4ccc5c(c4)c4c6oc7ccccc7c6ccc4n5-c4nc(-c5cccc6ccccc56)c5ccccc5n4)ccc32)cc1 ; c1ccc2cc(-c3ccc(-c4nc(-n5c6ccc(-c7ccc8sc9ccccc9c8c7)cc6c6c7sc8ccccc8c7ccc65)nc5ccccc45)cc3)ccc2c1]

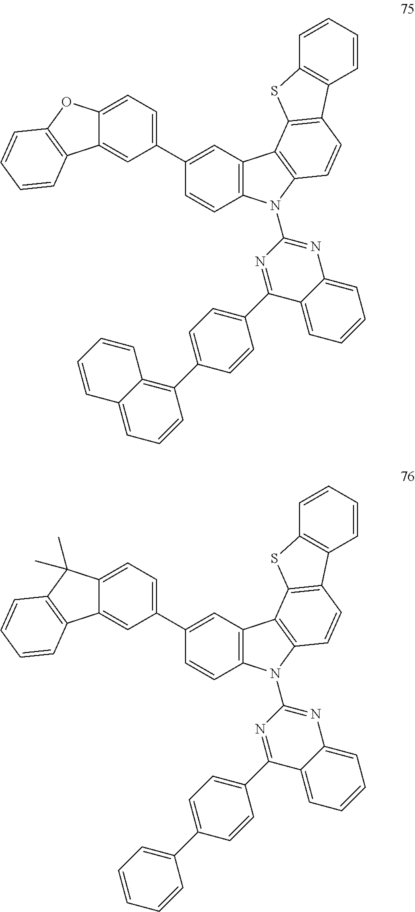 [SMILES: c1ccc2c(-c3ccc(-c4nc(-n5c6ccc(-c7ccc8oc9ccccc9c8c7)cc6c6c7sc8ccccc8c7ccc65)nc5ccccc45)cc3)cccc2c1 ; CC1(C)c2ccccc2-c2cc(-c3ccc4c(c3)c3c5sc6ccccc6c5ccc3n4-c3nc(-c4ccc(-c5ccccc5)cc4)c4ccccc4n3)ccc21]

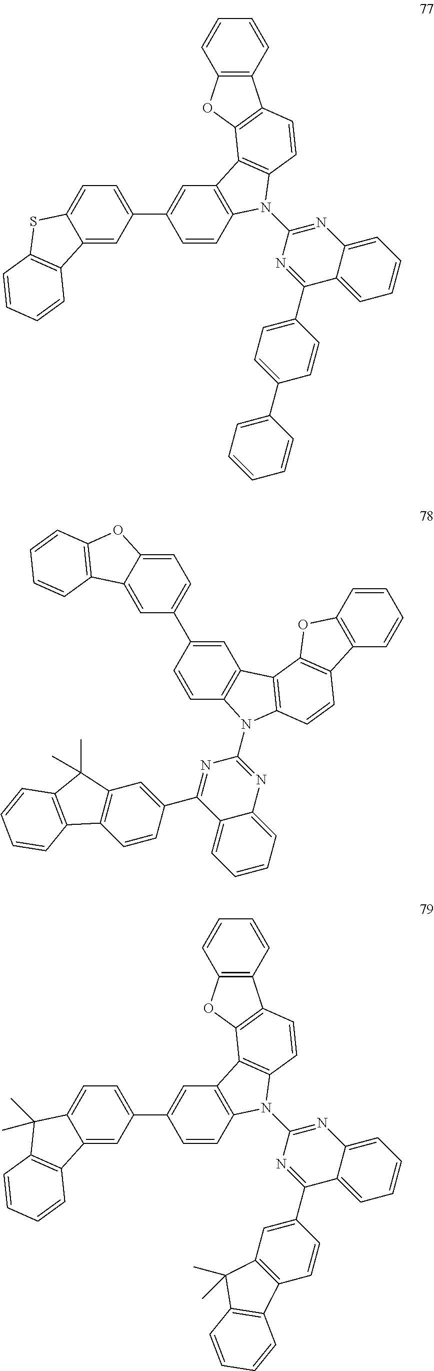

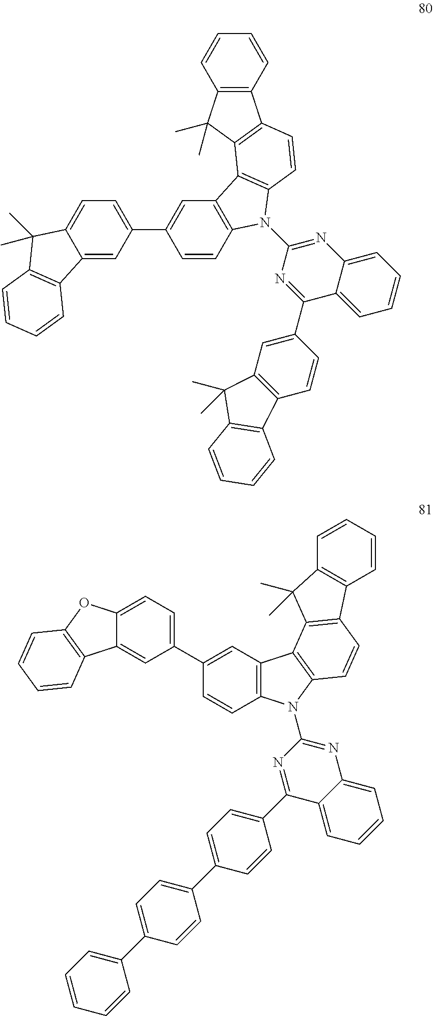 [SMILES: CC1(C)c2ccccc2-c2cc(-c3ccc4c(c3)c3c5c(ccc3n4-c3nc(-c4ccc6c(c4)C(C)(C)c4ccccc4-6)c4ccccc4n3)-c3ccccc3C5(C)C)ccc21 ; CC1(C)c2ccccc2-c2ccc3c(c21)c1cc(-c2ccc4oc5ccccc5c4c2)ccc1n3-c1nc(-c2ccc(-c3ccc(-c4ccccc4)cc3)cc2)c2ccccc2n1]

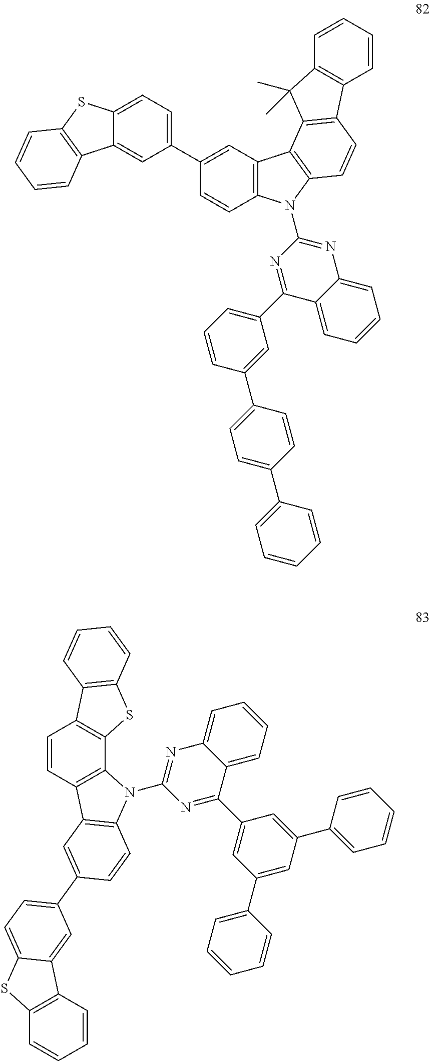 [SMILES: CC1(C)c2ccccc2-c2ccc3c(c21)c1cc(-c2ccc4sc5ccccc5c4c2)ccc1n3-c1nc(-c2cccc(-c3ccc(-c4ccccc4)cc3)c2)c2ccccc2n1 ; c1ccc(-c2cc(-c3ccccc3)cc(-c3nc(-n4c5ccc(-c6ccc7sc8ccccc8c7c6)cc5c5ccc6c7ccccc7sc6c54)nc4ccccc34)c2)cc1]

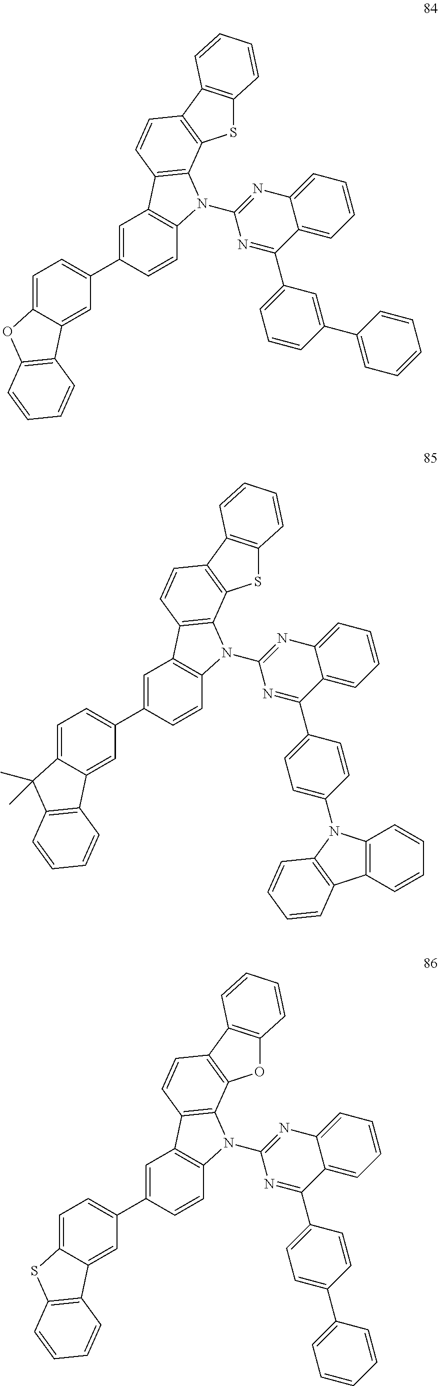 [SMILES: c1ccc(-c2cccc(-c3nc(-n4c5ccc(-c6ccc7oc8ccccc8c7c6)cc5c5ccc6c7ccccc7sc6c54)nc4ccccc34)c2)cc1 ; CC1(C)c2ccccc2-c2cc(-c3ccc4c(c3)c3ccc5c6ccccc6sc5c3n4-c3nc(-c4ccc(-n5c6ccccc6c6ccccc65)cc4)c4ccccc4n3)ccc21 ; c1ccc(-c2ccc(-c3nc(-n4c5ccc(-c6ccc7sc8ccccc8c7c6)cc5c5ccc6c7ccccc7oc6c54)nc4ccccc34)cc2)cc1]

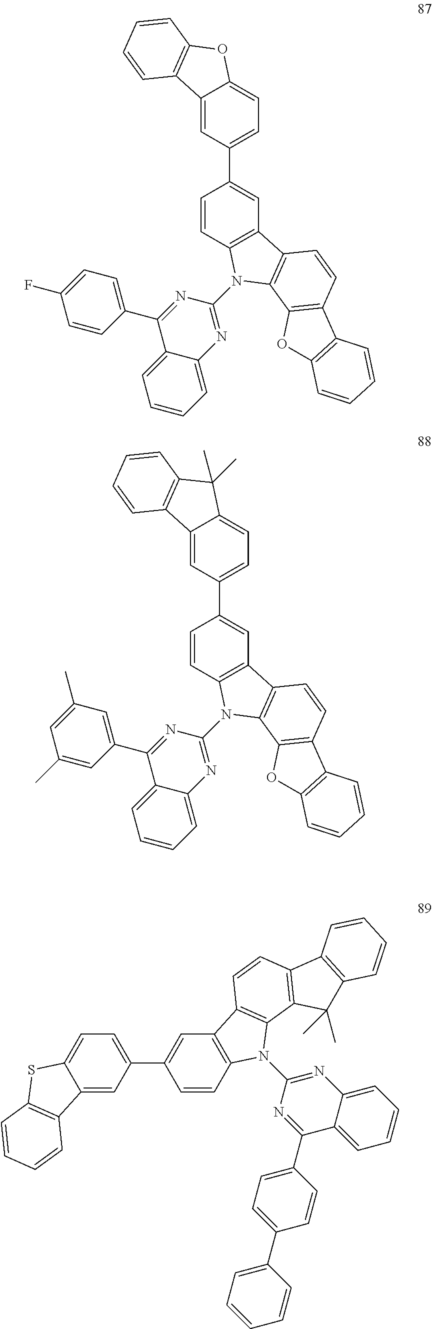

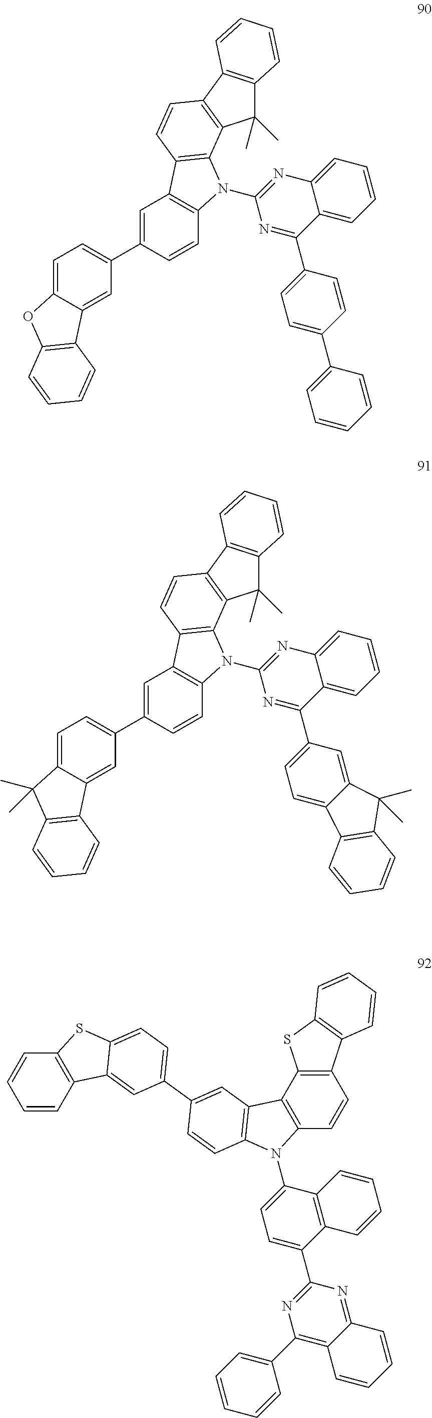

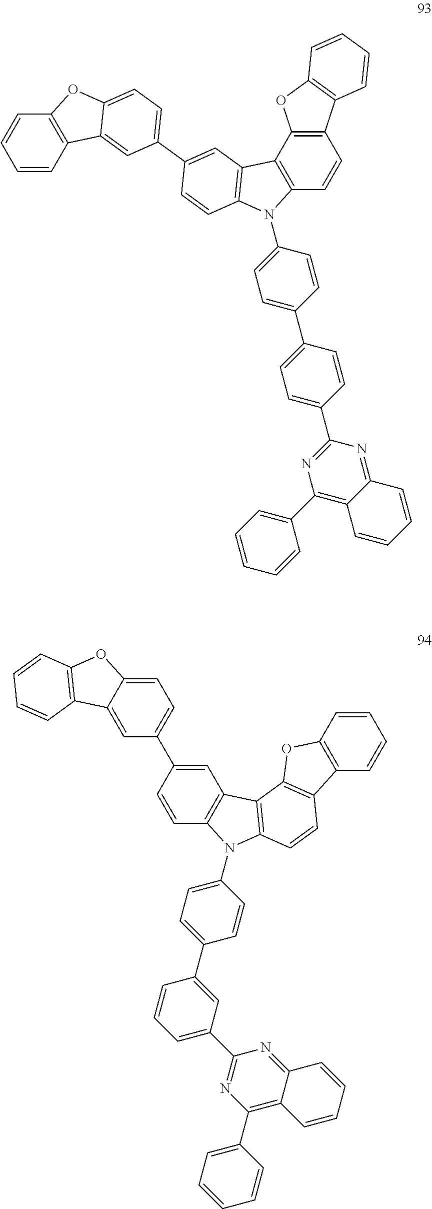 [SMILES: c1ccc(-c2nc(-c3ccc(-c4ccc(-n5c6ccc(-c7ccc8oc9ccccc9c8c7)cc6c6c7oc8ccccc8c7ccc65)cc4)cc3)nc3ccccc23)cc1 ; c1ccc(-c2nc(-c3cccc(-c4ccc(-n5c6ccc(-c7ccc8oc9ccccc9c8c7)cc6c6c7oc8ccccc8c7ccc65)cc4)c3)nc3ccccc23)cc1]

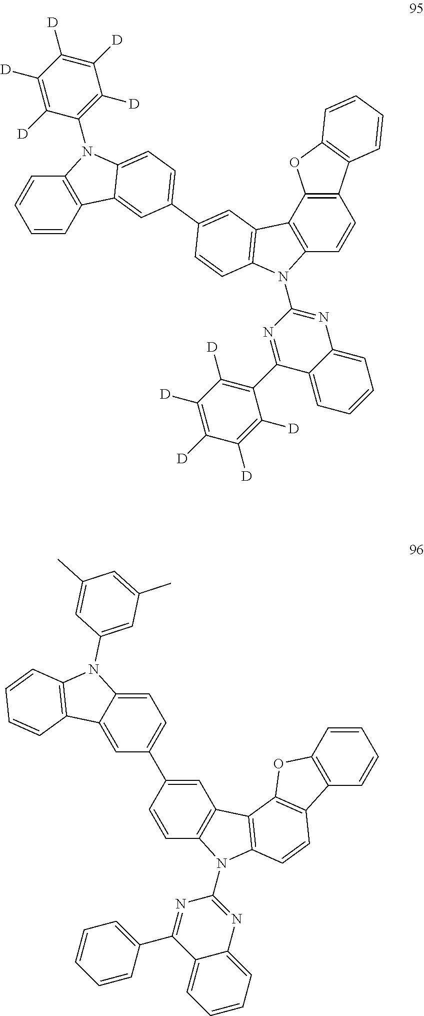 [SMILES: [2H]c1c([2H])c([2H])c(-c2nc(-n3c4ccc(-c5ccc6c(c5)c5ccccc5n6-c5c([2H])c([2H])c([2H])c([2H])c5[2H])cc4c4c5oc6ccccc6c5ccc43)nc3ccccc23)c([2H])c1[2H] ; Cc1cc(C)cc(-n2c3ccccc3c3cc(-c4ccc5c(c4)c4c6oc7ccccc7c6ccc4n5-c4nc(-c5ccccc5)c5ccccc5n4)ccc32)c1]

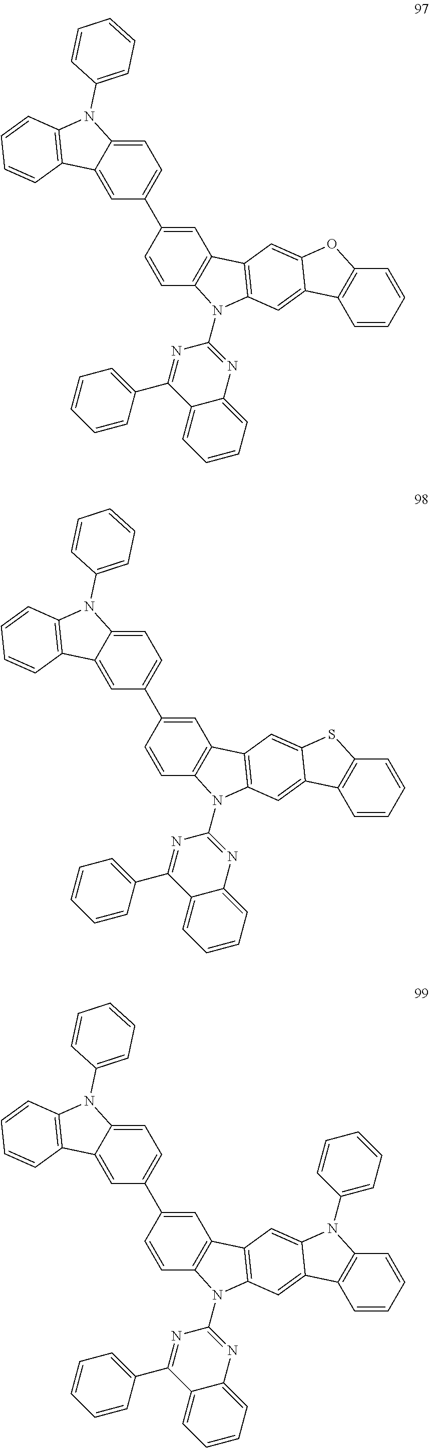 [SMILES: c1ccc(-c2nc(-n3c4ccc(-c5ccc6c(c5)c5ccccc5n6-c5ccccc5)cc4c4cc5oc6ccccc6c5cc43)nc3ccccc23)cc1 ; c1ccc(-c2nc(-n3c4ccc(-c5ccc6c(c5)c5ccccc5n6-c5ccccc5)cc4c4cc5sc6ccccc6c5cc43)nc3ccccc23)cc1 ; c1ccc(-c2nc(-n3c4ccc(-c5ccc6c(c5)c5ccccc5n6-c5ccccc5)cc4c4cc5c(cc43)c3ccccc3n5-c3ccccc3)nc3ccccc23)cc1]

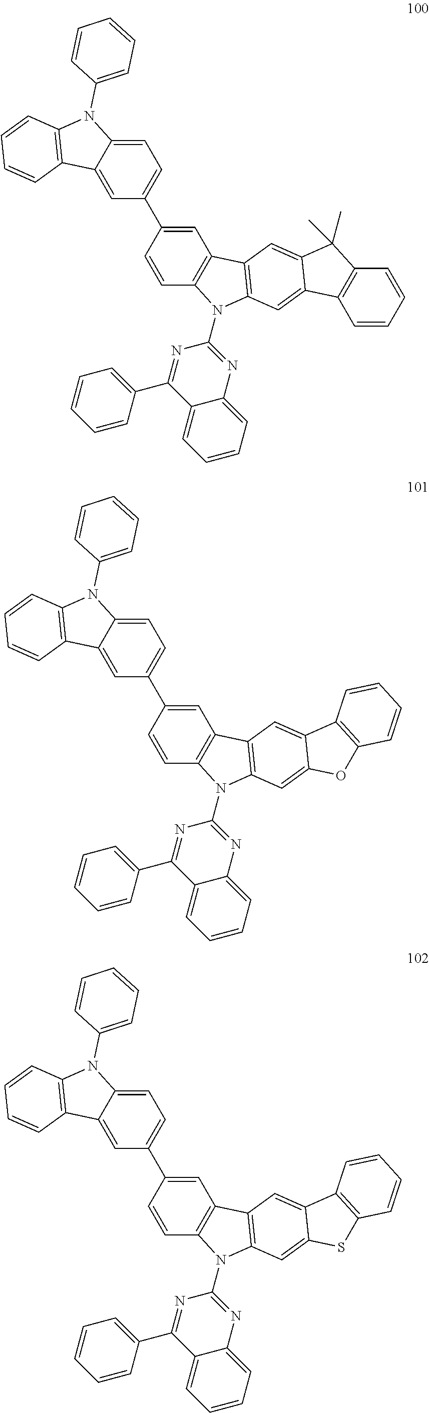 [SMILES: CC1(C)c2ccccc2-c2cc3c(cc21)c1cc(-c2ccc4c(c2)c2ccccc2n4-c2ccccc2)ccc1n3-c1nc(-c2ccccc2)c2ccccc2n1 ; c1ccc(-c2nc(-n3c4ccc(-c5ccc6c(c5)c5ccccc5n6-c5ccccc5)cc4c4cc5c(cc43)oc3ccccc35)nc3ccccc23)cc1 ; c1ccc(-c2nc(-n3c4ccc(-c5ccc6c(c5)c5ccccc5n6-c5ccccc5)cc4c4cc5c(cc43)sc3ccccc35)nc3ccccc23)cc1]

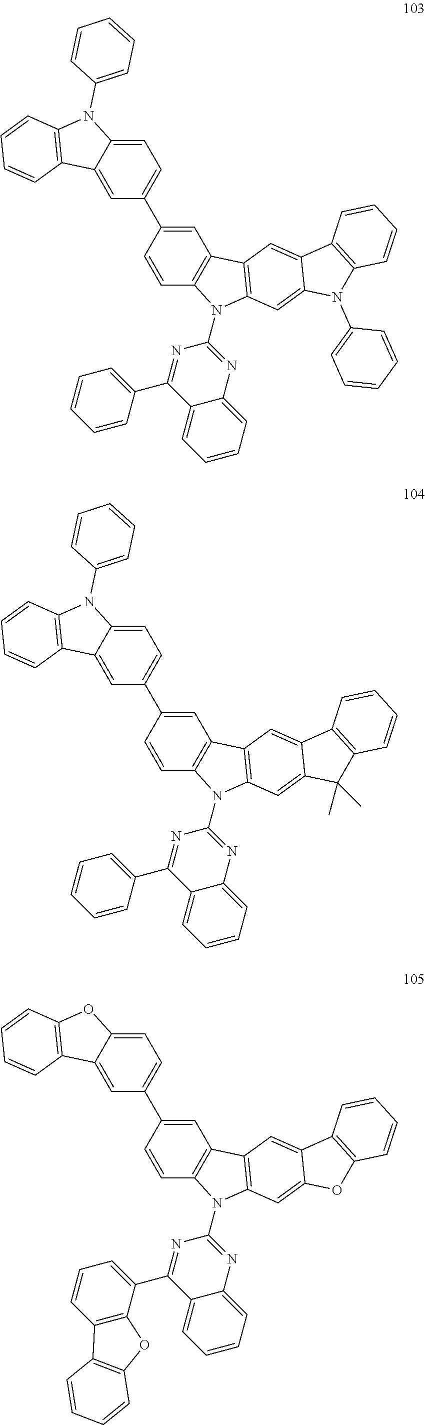 [SMILES: c1ccc(-c2nc(-n3c4ccc(-c5ccc6c(c5)c5ccccc5n6-c5ccccc5)cc4c4cc5c6ccccc6n(-c6ccccc6)c5cc43)nc3ccccc23)cc1 ; CC1(C)c2ccccc2-c2cc3c4cc(-c5ccc6c(c5)c5ccccc5n6-c5ccccc5)ccc4n(-c4nc(-c5ccccc5)c5ccccc5n4)c3cc21 ; c1ccc2c(-c3cccc4c3oc3ccccc34)nc(-n3c4ccc(-c5ccc6oc7ccccc7c6c5)cc4c4cc5c(cc43)oc3ccccc35)nc2c1]

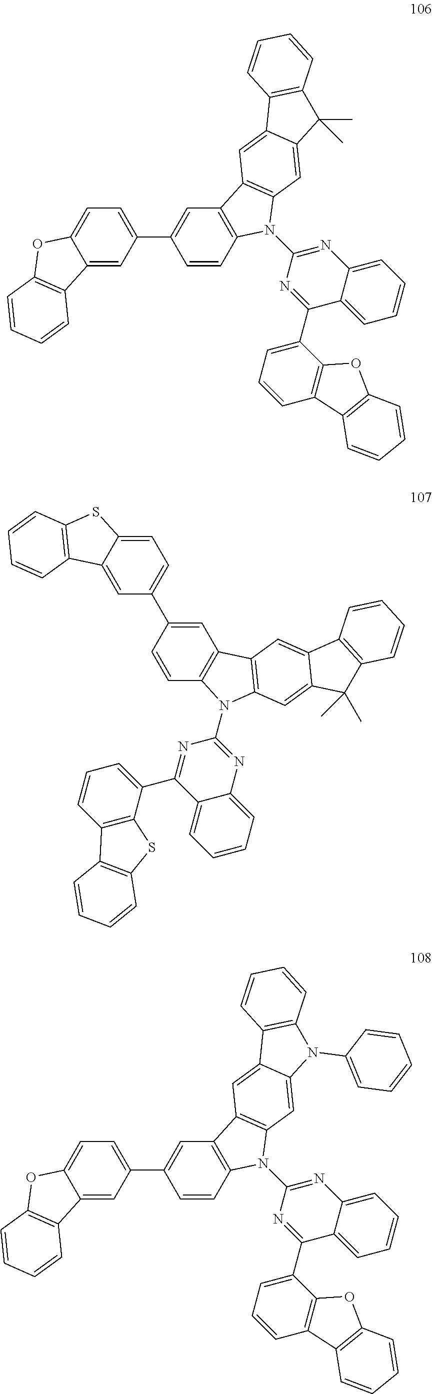 [SMILES: CC1(C)c2ccccc2-c2cc3c4cc(-c5ccc6oc7ccccc7c6c5)ccc4n(-c4nc(-c5cccc6c5oc5ccccc56)c5ccccc5n4)c3cc21 ; CC1(C)c2ccccc2-c2cc3c4cc(-c5ccc6sc7ccccc7c6c5)ccc4n(-c4nc(-c5cccc6c5sc5ccccc56)c5ccccc5n4)c3cc21 ; c1ccc(-n2c3ccccc3c3cc4c5cc(-c6ccc7oc8ccccc8c7c6)ccc5n(-c5nc(-c6cccc7c6oc6ccccc67)c6ccccc6n5)c4cc32)cc1]

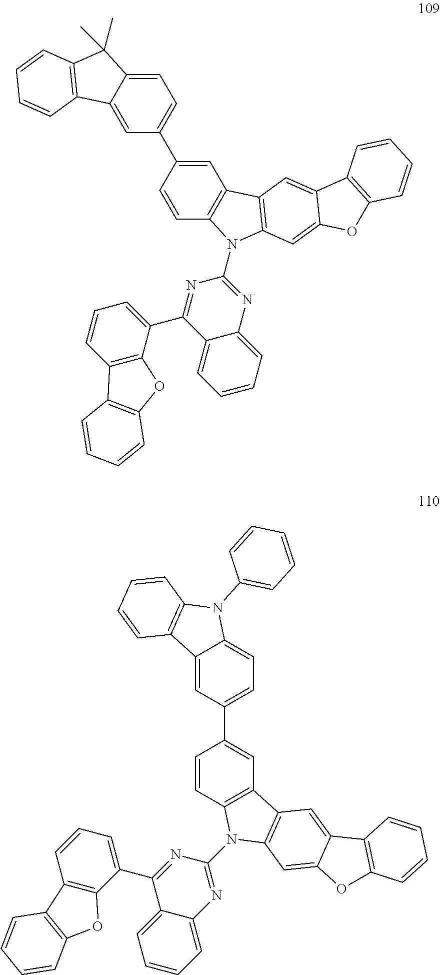 [SMILES: CC1(C)c2ccccc2-c2cc(-c3ccc4c(c3)c3cc5c(cc3n4-c3nc(-c4cccc6c4oc4ccccc46)c4ccccc4n3)oc3ccccc35)ccc21 ; c1ccc(-n2c3ccccc3c3cc(-c4ccc5c(c4)c4cc6c(cc4n5-c4nc(-c5cccc7c5oc5ccccc57)c5ccccc5n4)oc4ccccc46)ccc32)cc1]

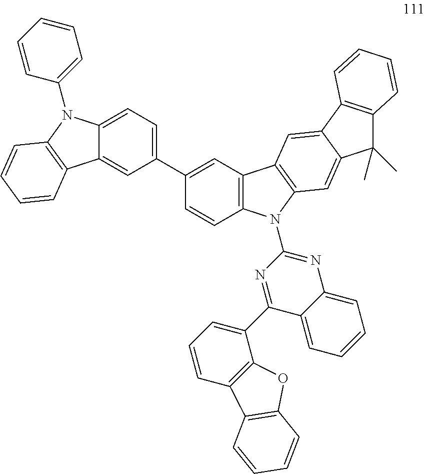 [SMILES: CC1(C)c2ccccc2-c2cc3c4cc(-c5ccc6c(c5)c5ccccc5n6-c5ccccc5)ccc4n(-c4nc(-c5cccc6c5oc5ccccc56)c5ccccc5n4)c3cc21]

The compound for an organic electronic material according to the present invention may be prepared as shown in the scheme below.

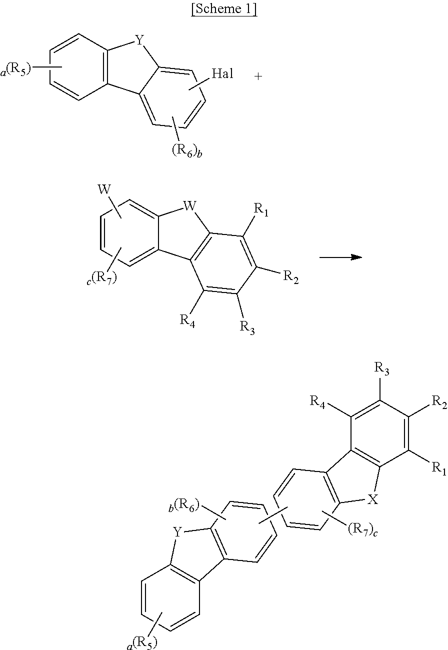

[In Scheme 1, X and Y, R₁ through R₇, and a through c are the same as defined in Chemical Formula 1, and Hal represents halogen and W represents hydrogen.]

Further, the present invention provides an organic electroluminescent device, and the organic electroluminescent device according to the present invention is characterized by including: a first electrode; a second electrode; and one or more organic material layers interposed between the first electrode and the second electrode. Here, the organic material layer may include one or more compounds for an organic electronic material of Chemical Formula 1. The organic material layer may include a luminescent layer, and the compound for an organic electronic material of Chemical Formula 1 is used as a host material in the luminescent layer.

When the compound for an organic electronic material of Chemical Formula 1 is used as a host material in the luminescent layer, one or more phosphorescent dopants may be included therein. The phosphorescent dopants used in the organic electroluminescent device of the present invention are not particularly limited, but a metal included in the phosphorescent dopants used in the organic electroluminescent device of the present invention is preferably selected from Ir, Pt and Cu.

Specifically, the compounds below are preferably used as compounds for the phosphorescent dopant.

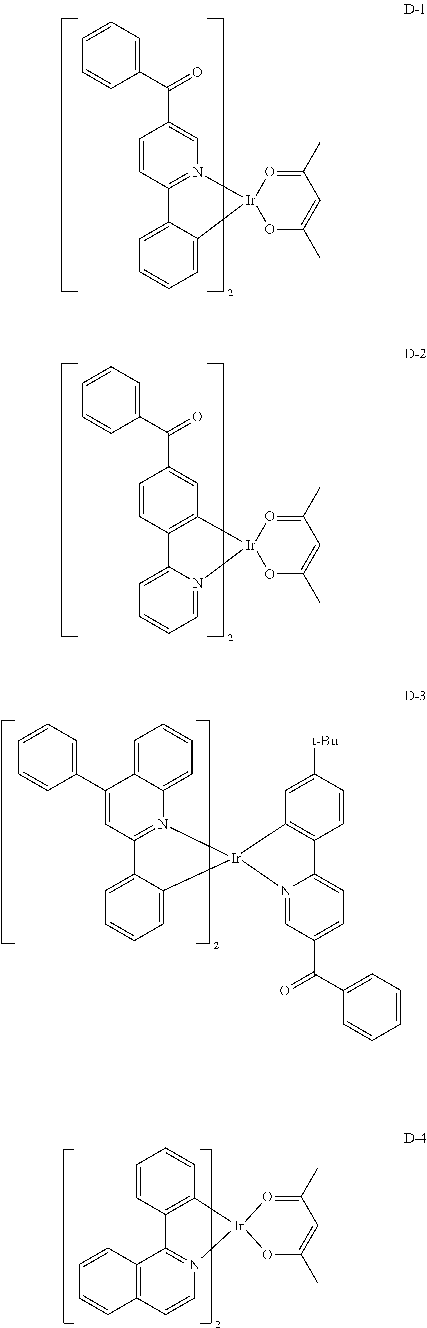

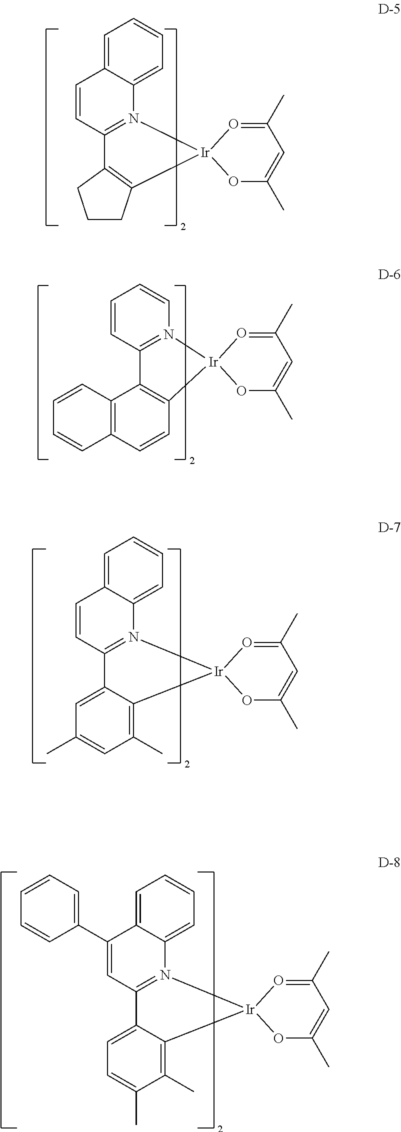

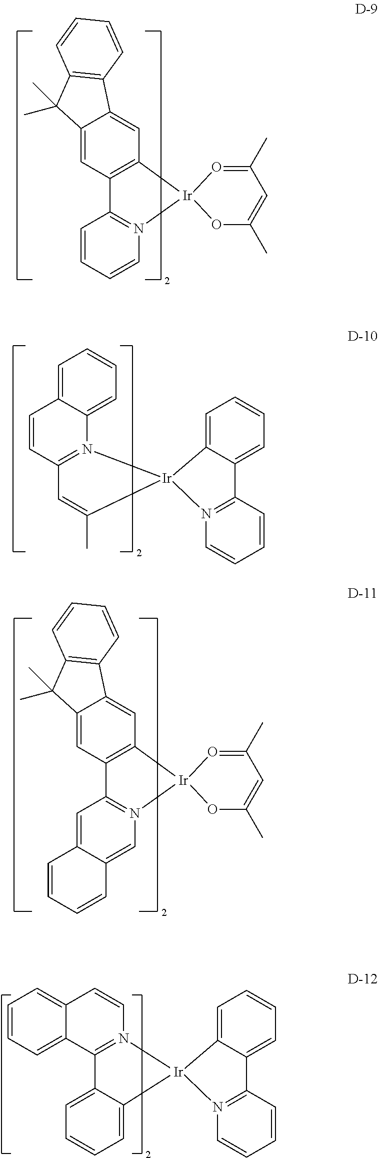

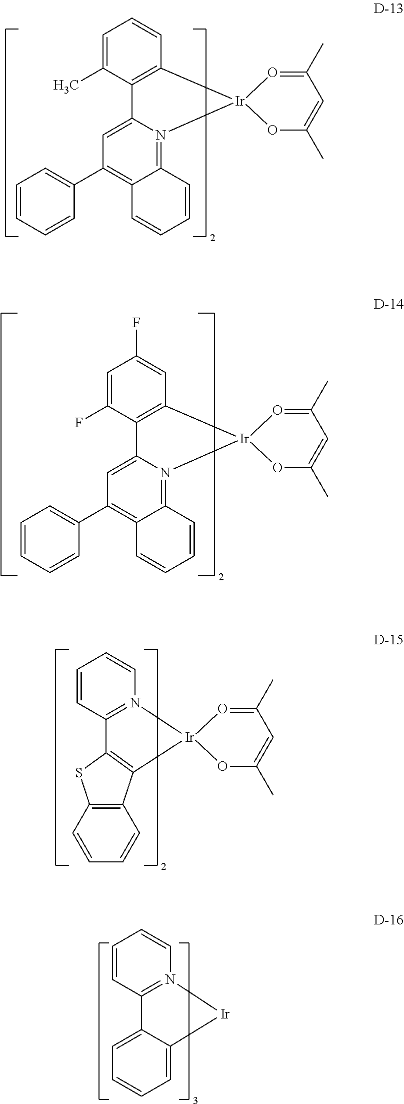

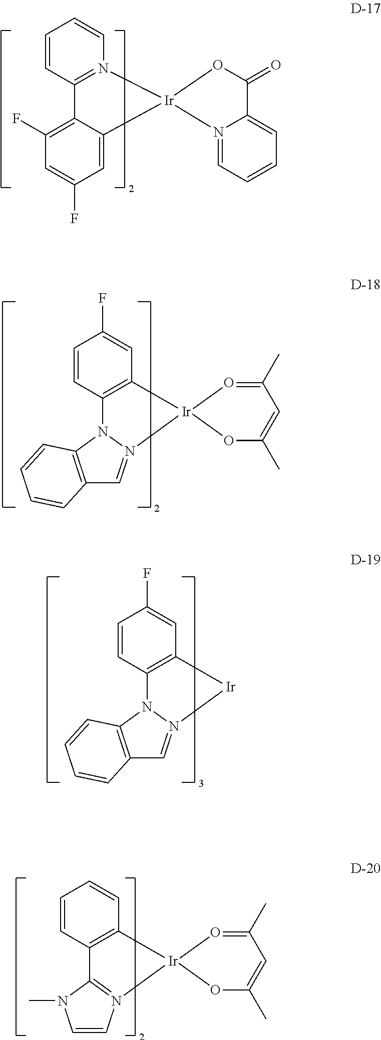

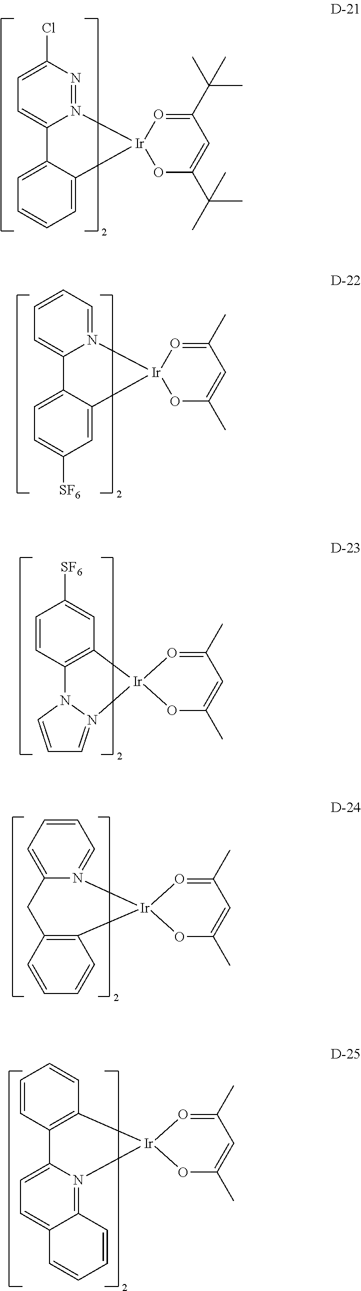

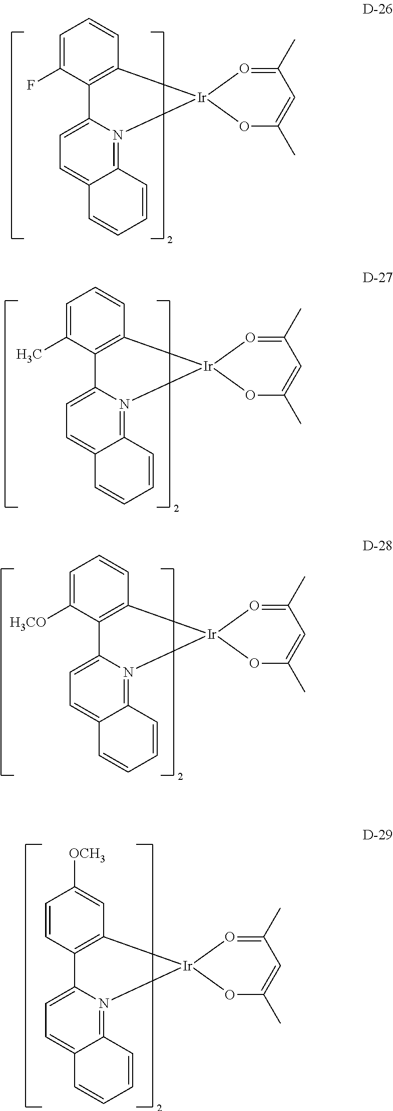

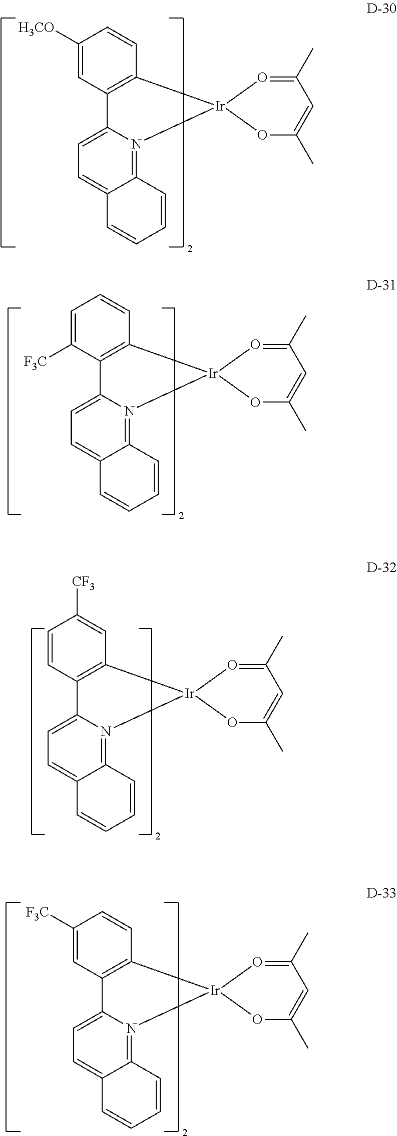

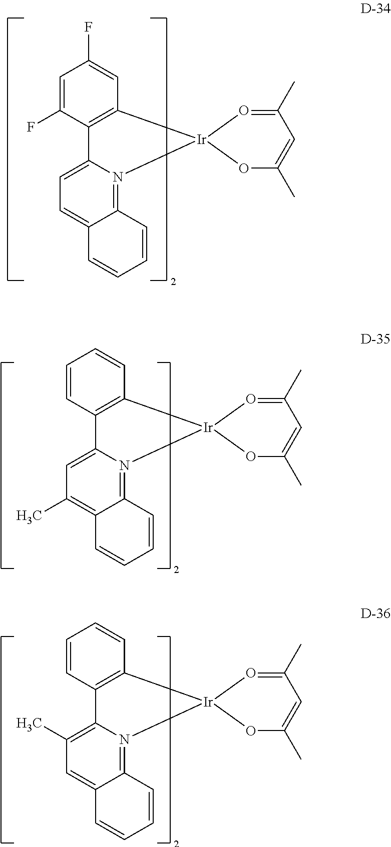

The organic electroluminescent device of the present invention may include the compound for an organic electronic material of Chemical Formula 1, and may further include one or more compounds selected from the group consisting of arylamine-based compounds or styrylarylamine-based compounds. The arylamine-based compounds or styrylarylamine-based compounds are exemplified in Korean Patent Application Nos. 10-2008-0123276, 10-2008-0107606, or 10-2008-0118428, but are not limited thereto.

Further, in the organic electroluminescent device of the present invention, the organic material layer may further include one or more metals or complex compounds selected from the group consisting of organic metals of Group I, Group II, 4th and 5th period transition metals, lanthanide metals, and d-transition elements, in addition to the compound for an organic electronic material of Chemical Formula 1, and the organic material layer may include a luminescent layer and a charge generating layer.

Further, the organic material layer may further include one or more organic luminescent layers including red, green, or blue luminescent compound at the same time, in addition to the compound for an organic electronic material, thereby manufacturing an organic electroluminescent device for emitting white light. The red, green, or blue luminescent compound is exemplified in Korean Patent Application Nos. 10-2008-0123276, 10-2008-0107606, or 10-2008-0118428, but not limited thereto.

In the organic electroluminescent device of the present invention, it is preferable to dispose at least one layer (hereinafter, referred to as “surface layer”), which selected from chalcogenide layers, metal halide layers, and metal oxide layers, on the inside surface of at least one side of a pair of electrodes. Specifically, it is preferable to dispose a metal chalcogenide (including oxides) layer of silicon and aluminum on an anode surface of a luminescent medium layer, and a metal halide layer or a metal oxide layer on a cathode surface of the luminescent medium layer. Stability of driving can be obtained by these. Preferable examples of the chalcogenides may include SiOx (1≦X≦2), AlOX (1≦X≦1.5), SiON, SiAlON, and the like, preferable examples of the metal halides may include LiF, MgF2, CaF2, rare earth metal fluoride, and the like, and preferable examples of the metal oxides may include Cs2O, Li2O, MgO, SrO, BaO, CaO and the like.

In the organic electroluminescent device of the present invention, it is also preferable to arrange a mixed region of an electron transport compound and a reductive dopant or a mixed region of a hole transport compound and an oxidative dopant on a surface of at least one of the pair of electrodes thus manufactured. Through this manner, the electron transport compound is reduced to an anion, which facilitates to inject or transport electrons into luminescent medium from the mixed region. In addition, the hole transport compound is oxidized into a cation, which facilitates to inject or transport holes into the luminescent medium from the mixed region. Preferable examples of the oxidative dopant may include various kinds of Lewis acid and acceptor compounds, and preferable examples of the reductive dopant may include alkaline metals, alkaline metal compounds, alkaline earth metals, rare metals, and a mixture thereof. Further, a layer of the reductive dopant may be used as the charge generating layer to manufacture a white organic electroluminescent device having two or more luminescent layers.

### Advantageous Effects

The compound for an organic electronic material according to the present invention has high electron transport efficiency, thereby preventing crystallization at the time of manufacture of a device, and allows a layer to be easily formed, thereby improving current characteristics of the device, and thus an OLED device having lowered driving voltage and improved power efficiency as well as superior luminous efficiency and lifespan characteristics can be manufactured.

### MODE OF THE INVENTION

Hereinafter, the present invention is further described by taking representative compounds of the present invention as examples with respect to the compound for an organic electronic material according to the invention, a preparing method thereof, and luminescent properties of a device, but those examples are provided only for illustration of the embodiments, and not intended to limit the scope of the invention.

### Preparation Example 1

### Preparation of Compound 1

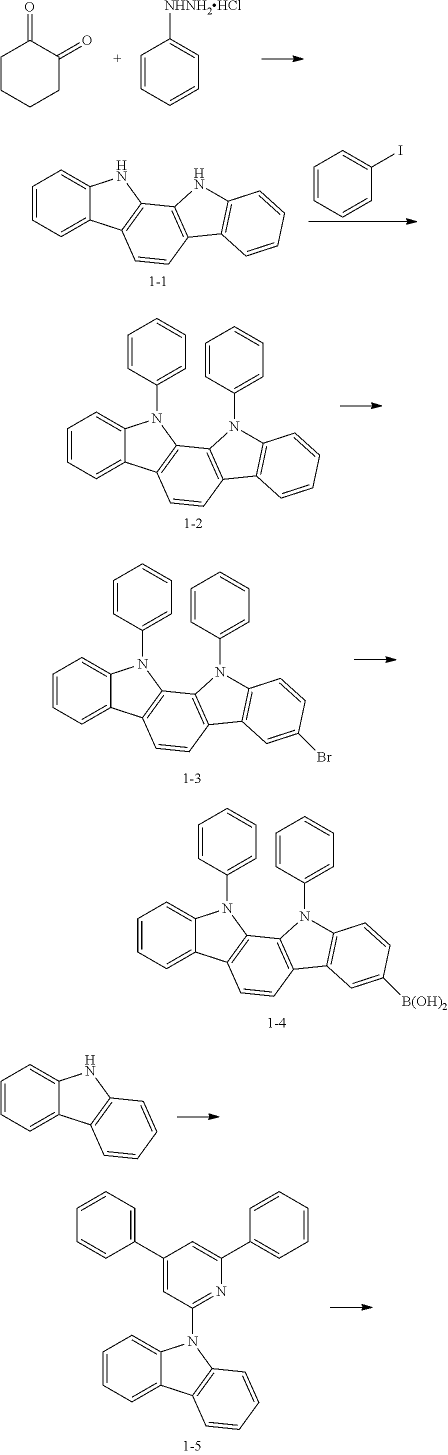

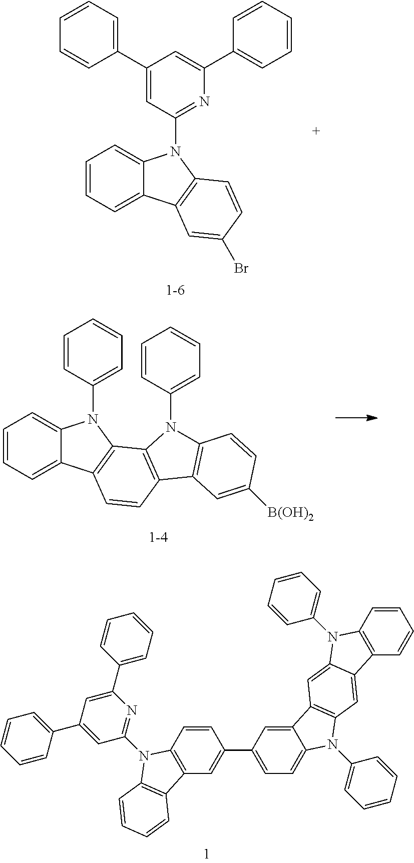 [SMILES: Brc1ccc2c(c1)c1ccccc1n2-c1cc(-c2ccccc2)cc(-c2ccccc2)n1 ; OB(O)c1ccc2c(c1)c1ccc3c4ccccc4n(-c4ccccc4)c3c1n2-c1ccccc1 ; c1ccc(-c2cc(-c3ccccc3)nc(-n3c4ccccc4c4cc(-c5ccc6c(c5)c5cc7c(cc5n6-c5ccccc5)c5ccccc5n7-c5ccccc5)ccc43)c2)cc1]

### Preparation of Compound 1-1

Cyclohexane-1,2-dione 25 g (0.22 mol) was put into a 1 L 2-neck round-bottom flask, and phenylhydrazine 70.9 g (0.49 mol), H₂SO₄ 1.18 mL (0.02 mol), and MeOH 720 mL (0.3M) were added thereinto, followed by heating to 100° C. Upon completion of the reaction after stirring for 4 hours, the resultant material was cooled down, and the thus generated solid was filtered and then washed with MeOH. The washed solid was put into a 1 L 2-neck round-bottom flask, and TFA (trifluoroacetic acid) 60 g and AcOH 600 mL were put thereinto, followed by heating and stirring at 100° C. for 12 hours. Upon completion of the reaction, washing with distilled water and then extraction with ethyl acetate were performed. The organic layer was dried over MgSO₄ and the solvent was removed by a rotary evaporator, followed by purification using column chromatography, thereby obtaining Compound 1-1 17 g (29%).

### Preparation of Compound 1-2

Compound 1-1 15 g (0.058 mol) was put into a 1 L 2-neck round-bottom flask, and iodobenzene 47 g (0.23 mol), CuI 33 g (0.17 mol), Cs₂CO₃ 76 g (0.23 mol), and quinoline 650 mL were added thereinto, followed by heating and stirring at 190° C. Upon completion of the reaction, the solvent was removed by using a distilling apparatus. Afterthat, washing with distilled water and then extraction with ethyl acetate were performed. The organic layer was dried over MgSO₄ and the solvent was removed by a rotary evaporator, followed by purification using column chromatography, thereby obtaining Compound 1-2 15 g (65%).

### Preparation of Compound 1-3

Compound 1-2 6.6 g (0.016 mol) was put into a 1 L 2-neck round-bottom flask, and NBS (N-bromosuccinimide) 3.3 g (0.018 mol) and THF (tetrahydrofuran) 300 mL were added thereinto, followed by stirring under reflux for 12 hours. Upon completion of the reaction, washing with distilled water and then extraction with ethyl acetate were performed. The organic layer was dried over MgSO₄ and the solvent was removed by a rotary evaporator, followed by washing with hexane, thereby obtaining Compound 1-3 7.2 g (92%).

### Preparation of Compound 1-4

Compound 1-3 4.8 g (0.010 mol) was put into a 500 mL round-bottom flask, and dried under vacuum. Then, the resultant flask was filled with nitrogen gas, and THF 100 Ml was put thereinto. The resultant material was cooled to −78° C. n-Butyllithium (2.5M) 6.2 mL (0.015 mol) was slowly added thereinto, followed by stirring for 1 hour while maintaining low temperature. Then, B(OMe)3 1.7 mL (0.015 mmol) was added thereinto at −78° C., followed by stirring under reflux for 12 hours. Upon completion of the reaction, 1M HCl was added thereinto. After 10 minutes, washing with distilled water and then extraction with ethylacetate were performed. The organic layer was dried over MgSO₄ and the solvent was removed by a rotary evaporator, followed by purification using column chromatography, thereby obtaining Compound 1-4 2.8 g (62%).

### Preparation of Compound 1-5

7H-benzo[c]carbazole 8.9 g (41.10 mmol), 2-chloro-4,6-diphenylpyridine 13.1 g (49.32 mmol), Pd(OAc)₂ 0.46 g, NaOt-bu 7.9 g (82.20 mmol), toluene 100 mL, and P(t-bu)₃ 2 mL (4.11 mmol, 50% in toluene) were put, followed by stirring under reflux. After 10 hours, the mixture was cooled to room temperature, and distilled water was added thereinto, followed by extraction with EA. Then, drying over anhydrous MgSO₄ and then drying under reduced pressure were performed, followed by column separation, thereby obtaining Compound 1-5 13.2 g (81%).

### Preparation of Compound 1-6

Compound 1-5 13.5 g (33.98 mmol) was put into a 1-neck flask, which was treated under vacuum and filled with argon. THF 500 mL was put thereinto, followed by stirring at 0° C. for 10 minutes. NBS 7.35 g (40.78 mmol) was added thereinto, followed by stirring at room temperature for one day. Upon completion of the reaction, extraction with distilled water and EA was performed. The organic layer was dried over anhydrous MgSO₄ and the solvent was removed by a rotary evaporator, and then column chromatography using hexane and EA as development solvent was performed, thereby obtaining Compound 1-6 13.8 g (82%).

### Preparation of Compound 1

Compound 1-4 7.9 g (17.4 mmol), Compound 1-6 9.9 g (20.88 mmol), Pd(PPh₃)₄ 0.8 g (0.7 mmol), 2M aqueous K₂CO₃ solution 20 mL, toluene 100 mL, and ethanol 50 mL were put, followed by stirring under reflux for 12 hours. Then, washing with distilled water and then extraction with EA were performed. Then, drying over anhydrous MgSO₄ and then distillation under reduced pressure were performed, followed by column separation, thereby obtaining Compound 1-8, 6 g (10.7 mmol, 62%).

MS/EIMS found 803, calculated 802.96

### Preparation Example 2

### Preparation of Compound 2

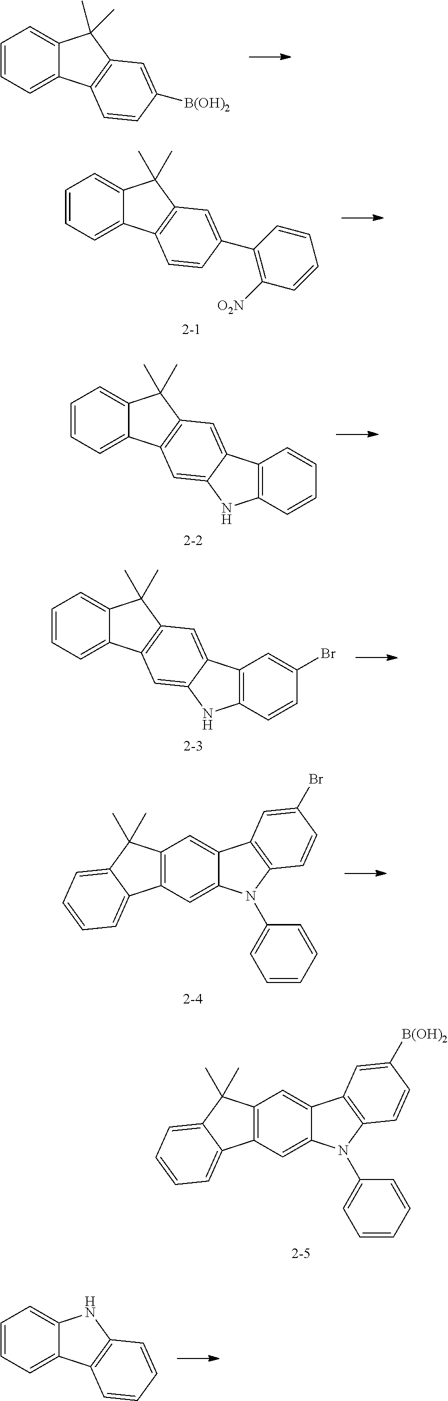

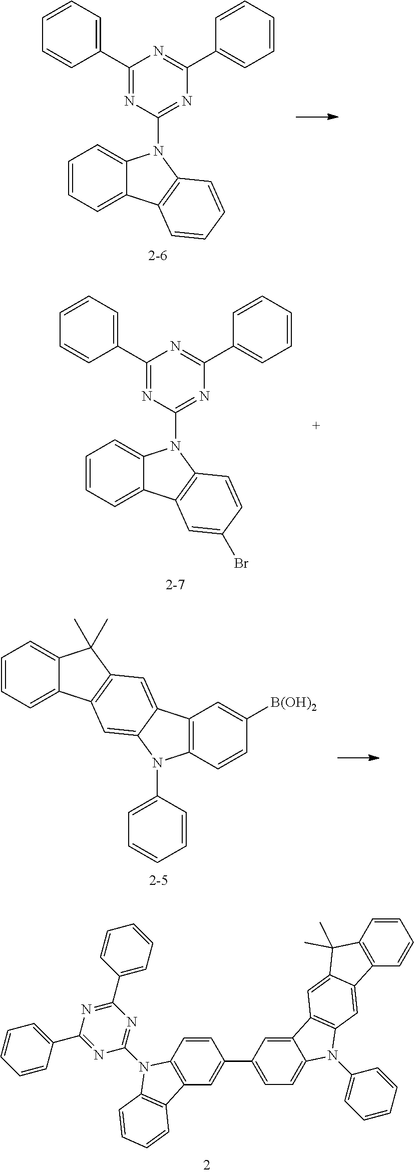 [SMILES: c1ccc(-c2nc(-c3ccccc3)nc(-n3c4ccccc4c4ccccc43)n2)cc1 ; Brc1ccc2c(c1)c1ccccc1n2-c1nc(-c2ccccc2)nc(-c2ccccc2)n1 ; CC1(C)c2ccccc2-c2cc3c(cc21)c1cc(B(O)O)ccc1n3-c1ccccc1 ; CC1(C)c2ccccc2-c2cc3c(cc21)c1cc(-c2ccc4c(c2)c2ccccc2n4-c2nc(-c4ccccc4)nc(-c4ccccc4)n2)ccc1n3-c1ccccc1]

### Preparation of Compound 2-1

1-bromo-2-nitrobenzene 15 g (0.074 mol) was put into a 1 L 2-neck round bottom flask, and then 9,9-dimethyl-9H-fluoren-2-ylboronic acid 23 g (0.096 mol), Pd(PPh₃)₄ 4.2 g (0.003 mol), Na₂CO₃ (2M) 111 mL, and ethanol 111 mL were put thereinto. Then, toluene 200 mL was added thereinto, followed by heating and stirring at 120° C. for 3 hours. Upon completion of the reaction, washing with distilled water and then extraction with ethyl acetate were performed. The organic layer was dried over MgSO₄ and the solvent was removed by a rotary evaporator, followed by purification using column chromatography, thereby obtaining Compound 2-1 22 g (95%).

### Preparation of Compound 2-2

Compound 2-1 24 g (0.076 mol) was put into a 1 L 2-neck round bottom flask, then triethylphosphite 200 mL and 1,2-dichlorobenzene 200 mL were added thereinto, followed by heating and stirring at 140° C. for 12 hours. Upon completion of the reaction, the solvent was distilled, and then washing with distilled water and extraction with ethyl acetate were performed. The organic layer was dried over MgSO₄ and the solvent was removed by a rotary evaporator, followed by purification using column chromatography, thereby obtaining Compound 2-2 7 g (33%).

### Preparation of Compound 2-3

Compound 2-2 8.1 g (0.028 mol) was put into a 1 L 2-neck round bottom flask, and then DMF (Dimethylformamide) 300 mL was put thereinto, followed by stirring under reflux at 0° C. for 10 minutes. NBS 5.08 g (0.028 mol) was dissolved in DMF 300 mL, and this was slowly added into the resultant material, followed by stirring under reflux at 0° C. for 6 hours. Upon completion of the reaction, the resultant material was neutralized with distilled water and extracted with ethyl acetate. The organic layer was dried over MgSO₄ and the solvent was removed by a rotary evaporator, and then column chromatography using ethylacetate as a development solvent was performed, thereby obtaining Compound 2-3 9 g (87%).

### Preparation of Compound 2-4

Compound 2-3 9 g (0.024 mol) was put into a 3 L 2-neck round bottom flask, and iodobenzene 6 g (0.029 mol), Pd(OAc)₂ 123 mg (0.0005 mol), P(t-Bu)₃ 50% 0.5 mL (0.002 mmol) and NaOt-Bu 4.7 g (0.049 mol) were added thereinto. After forming of vacuum condition, toluene 200 mL was put thereinto under a nitrogen atmosphere, followed by stirring at 120° C. for 12 hours. Upon completion of the reaction, the resultant material was neutralized with distilled water and extracted with ethyl acetate. The organic layer was dried over MgSO₄ and the solvent was removed by a rotary evaporator, and then column chromatography using ethylacetate as a development solvent was performed, thereby obtaining Compound 2-4 8 g (74%).

### Preparation of Compound 2-5

Compound 2-4 4.4 g (0.010 mol) was put into a 500 mL round-bottom flask, which was then dried under vacuum. Then, the resultant flask was filled with nitrogen gas, and THF 100 mL was put thereinto. The resultant material was cooled to −78° C. n-Butyllithium (2.5M) 6.2 mL (0.015 mol) was slowly added thereinto, followed by stirring under reflux for 1 hour while maintaining low temperature. Then, B(OMe)₃ 1.7 mL (0.015 mmol) was added thereinto at −78° C., followed by stirring for 12 hours. Upon completion of the reaction, 1M HCl was added into the resultant material, and, after 10 minutes, washing with distilled water and then extraction with ethylacetate were performed. The organic layer was dried over MgSO₄ and the solvent was removed by a rotary evaporator, followed by purification using column chromatography, thereby obtaining Compound 2-5 2.3 g (56%).

### Preparation of Compound 2-6

The same method as the preparation of Compound 1-5 was conducted by using 7H-benzo[c]carbazole 8.9 g (41.10 mmol), and 2-chloro-4,6-diphenyl-1,3,5-triazine 13.2 g (49.32 mmol), thereby obtaining Compound 2-6 14.2 g (87%).

### Preparation of Compound 2-7

The same method as the preparation of Compound 1-6 was conducted by using Compound 2-6 14.2 g (35.64 mmol), thereby obtaining Compound 2-7 14.6 g (86%).

### Preparation of Compound 2

The same method as the preparation of Compound 1 was conducted by using Compound 2-5 7.0 g (17.4 mmol) and Compound 2-7 9.9 g (20.88 mmol), thereby obtaining Compound 27.5 g (9.92 mmol, 57%).

MS/EIMS found 756, calculated 755.91

### Preparation Example 3

### Preparation of Compound 3

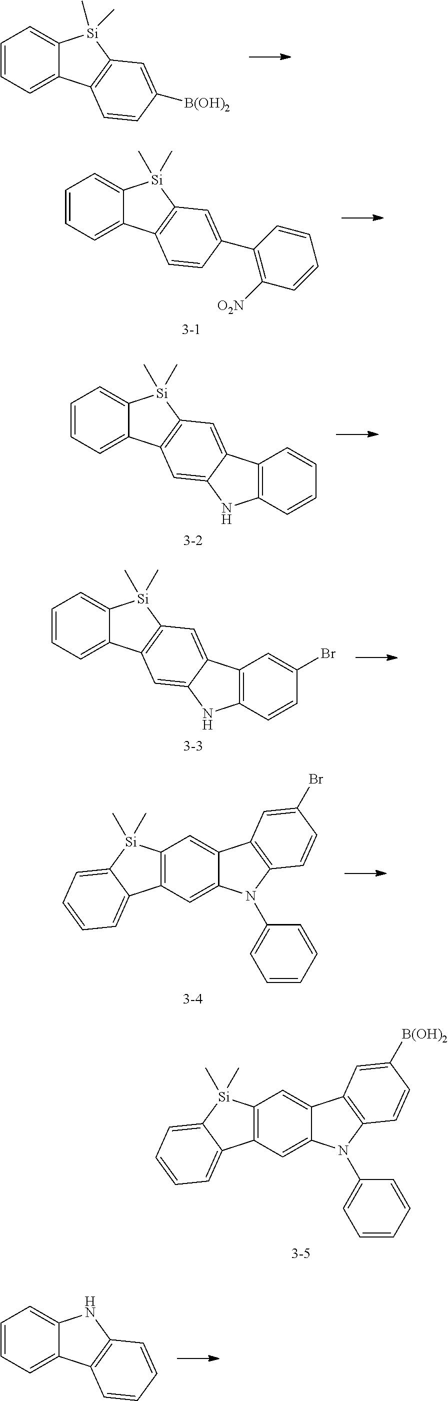

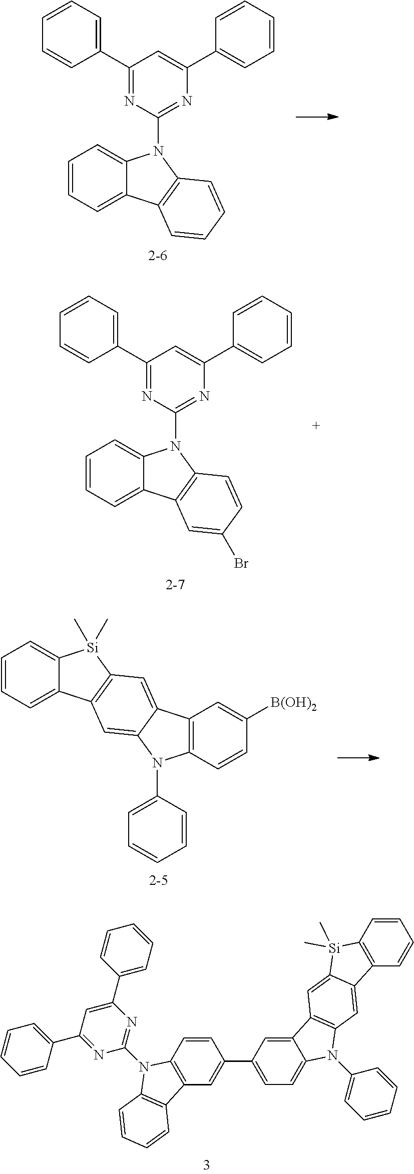 [SMILES: c1ccc(-c2cc(-c3ccccc3)nc(-n3c4ccccc4c4ccccc43)n2)cc1 ; Brc1ccc2c(c1)c1ccccc1n2-c1nc(-c2ccccc2)cc(-c2ccccc2)n1 ; C[Si]1(C)c2ccccc2-c2cc3c(cc21)c1cc(B(O)O)ccc1n3-c1ccccc1 ; C[Si]1(C)c2ccccc2-c2cc3c(cc21)c1cc(-c2ccc4c(c2)c2ccccc2n4-c2nc(-c4ccccc4)cc(-c4ccccc4)n2)ccc1n3-c1ccccc1]

### Preparation of Compound 3-1

1-bromo-2-nitrobenzene 15 g (0.074 mol) was put into a 1 L 2-neck round-bottom flask, and then, the same method as the preparation of Compound 2-1 by using 5,5-dimethyl-5H-dibenzo[b,d]silol-3-ylboronic acid 24.4 g (0.096 mol), thereby obtaining Compound 3-1 22.6 g (95%).

### Preparation of Compound 3-2

Compound 3-1 22.6 g (0.068 mol) was put into a 1 L 2-neck round-bottom flask, and then, the same method as the preparation of Compound 2-2 was conducted by using Compound 2-2, thereby obtaining Compound 3-2 7 g (35%).

### Preparation of Compound 3-3

Compound 3-2 8.4 g (0.028 mol) was put into a 1 L 2-neck round-bottom flask, and then, the same method as the preparation of Compound 2-3 was conducted by using Compound 2-3, thereby obtaining Compound 3-3 9.1 g (86%).

### Preparation of Compound 3-4

The same method as the preparation of Compound 2-4 was conducted by using Compound 3-3 9 g (0.024 mol) in a 3 L 2-neck round-bottom flask, thereby obtaining

Compound 3-4 7.7 g (71%).

### Preparation of Compound 3-5

Compound 3-4 4.5 g (0.010 mol) was put into a 500 mL round-bottom flask, and then, the same method as the preparation of Compound 2-5 was conducted by using Compound 2-5, thereby obtaining Compound 3-5 2.2 g (53%).

### Preparation of Compound 3-6

The same method as the preparation of Compound 1-5 was conducted by using 7H-benzo[c]carbazole 8.9 g (41.10 mmol) and 2-chloro-4,6-diphenylpyrimidine 13.2 g (49.32 mmol), thereby obtaining Compound 3-6 14.2 g (87%).

### Preparation of Compound 3-7

The same method as the preparation of Compound 1-6 was conducted by using Compound 3-6 14.2 g (35.64 mmol), thereby obtaining Compound 3-7 14.6 g (86%).

### Preparation of Compound 3

The same method as the preparation of Compound 1 was conducted by using Compound 3-5 7.3 g (17.4 mmol) and Compound 3-7 9.9 g (20.88 mmol), thereby obtaining Compound 37.1 g (9.2 mmol, 53%).

MS/EIMS found 771, calculated 770.99

### Preparation Example 4

### Preparation of Compound 4

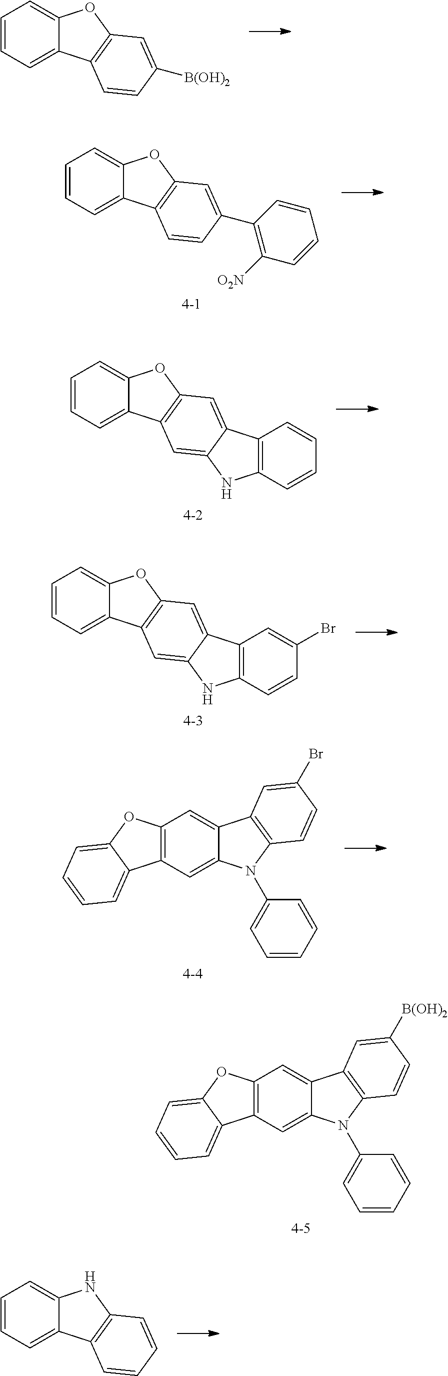

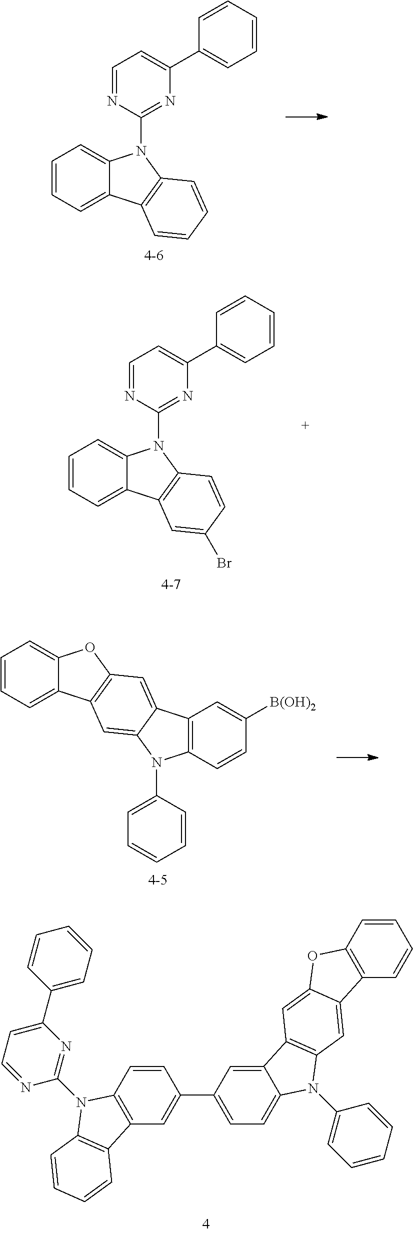 [SMILES: c1ccc(-c2ccnc(-n3c4ccccc4c4ccccc43)n2)cc1 ; Brc1ccc2c(c1)c1ccccc1n2-c1nccc(-c2ccccc2)n1 ; OB(O)c1ccc2c(c1)c1cc3oc4ccccc4c3cc1n2-c1ccccc1 ; c1ccc(-c2ccnc(-n3c4ccccc4c4cc(-c5ccc6c(c5)c5cc7oc8ccccc8c7cc5n6-c5ccccc5)ccc43)n2)cc1]

### Preparation of Compound 4-1

1-bromo-2-nitrobenzene 15 g (0.074 mol) was put into a 1 L 2-neck round-bottom flask, and then, the same method as the preparation of Compound 2-1 was conducted by using dibenzo[b,d]furan-3-ylboronic acid 20.3 g (0.096 mol), thereby obtaining Compound 4-1 18.4 g (86%).

### Preparation of Compound 4-2

Compound 4-1 19.7 g (0.068 mol) was put into a 1 L 2-neck round-bottom flask, and then, the same method as the preparation of Compound 2-2 was conducted by using Compound 2-2, thereby obtaining Compound 4-2 7.3 g (42%).

### Preparation of Compound 4-3

Compound 4-2 7.2 g (0.028 mol) was put into a 1 L 2-neck round-bottom flask, and then, the same method as the preparation of Compound 2-3 was conducted, thereby obtaining Compound 4-3 7.9 g (84%).

### Preparation of Compound 4-4

The same method as the preparation of Compound 2-4 was conducted by using Compound 4-3 8.1 g (0.024 mol) in a 3 L 2-neck round-bottom flask, thereby obtaining Compound 4-4 7.5 g (76%).

### Preparation of Compound 4-5

The same method as the preparation of Compound 2-5 was conducted by using Compound 4-4 4.1 g (0.010 mol) in a 500 mL round-bottom flask, thereby obtaining Compound 4-5 1.9 g (51%).

### Preparation of Compound 4-6

The same method as the preparation of Compound 1-5 was conducted by using 7H-benzo[c]carbazole 8.9 g (41.10 mmol) and 2-chloro-4-diphenylpyrimidine 9.4 g (49.32 mmol), thereby obtaining Compound 4-6 10.8 g (82%).

### Preparation of Compound 4-7

The same method as the preparation of Compound 1-6 was conducted by using Compound 4-6 11.5 g (35.64 mmol), thereby obtaining Compound 4-7 10.8 g (76%).

### Preparation of Compound 4

The same method as the preparation of Compound 1 was conducted by using Compound 4-5 6.6 g (17.4 mmol) and Compound 4-7 8.4 g (20.88 mmol), thereby obtaining Compound 45.6 g (8.6 mmol, 49%).

MS/EIMS found 653, calculated 652.74

### Preparation Example 5

### Preparation of Compound 7

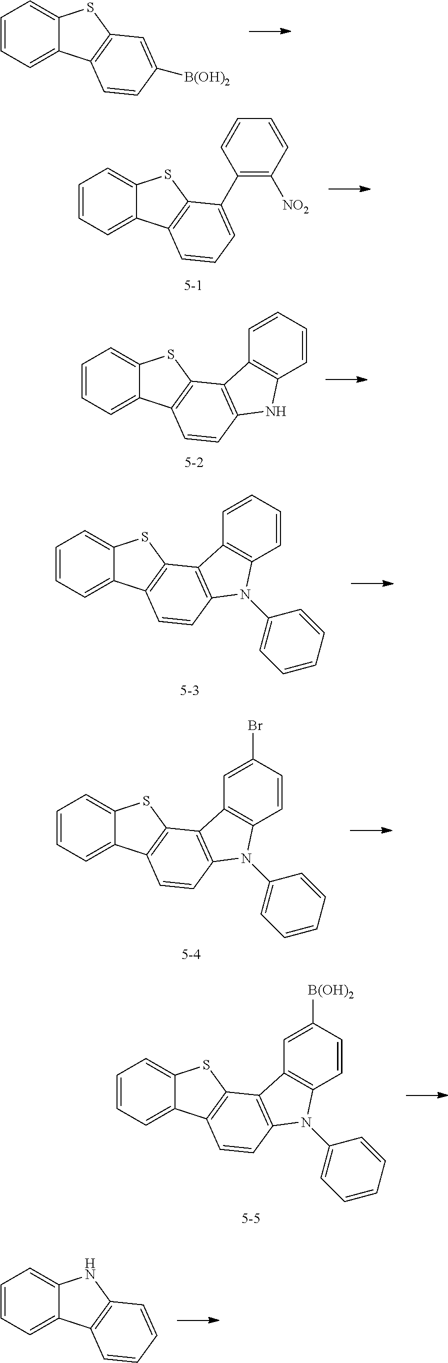

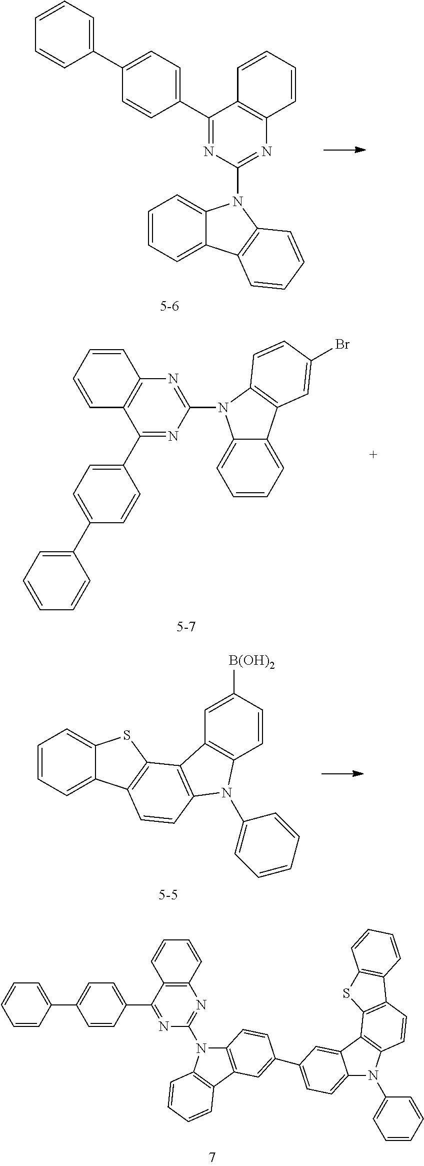 [SMILES: c1ccc(-c2ccc(-c3nc(-n4c5ccccc5c5ccccc54)nc4ccccc34)cc2)cc1 ; Brc1ccc2c(c1)c1ccccc1n2-c1nc(-c2ccc(-c3ccccc3)cc2)c2ccccc2n1 ; OB(O)c1ccc2c(c1)c1c3sc4ccccc4c3ccc1n2-c1ccccc1 ; c1ccc(-c2ccc(-c3nc(-n4c5ccccc5c5cc(-c6ccc7c(c6)c6c8sc9ccccc9c8ccc6n7-c6ccccc6)ccc54)nc4ccccc34)cc2)cc1]

### Preparation of Compound 5-1

1-bromo-2-nitrobenzene 15 g (0.074 mol) was put into a 1 L 2-neck round-bottom flask, and then, the same method as the preparation of Compound 2-1 was conducted by using dibenzo[b,d]thiophen-4-ylboronic acid 21.9 g (0.096 mol), thereby obtaining Compound 5-1 18.5 g (82%).

### Preparation of Compound 5-2

The same method as the preparation of Compound 2-2 was conducted by using Compound 5-1 20.8 g (0.068 mol) in a 1 L 2-neck round-bottom flask, thereby obtaining Compound 5-2 6.9 g (37%).

### Preparation of Compound 5-3

The same method as the preparation of Compound 2-3 was conducted by using Compound 5-2 7.7 g (0.028 mol) in a 1 L 2-neck round-bottom flask, thereby obtaining Compound 5-3 7.4 g (76%).

### Preparation of Compound 5-4

The same method as the preparation of Compound 2-4 was conducted by using Compound 5-3 8.4 g (0.024 mol) in a 3 L 2-neck round-bottom flask, thereby obtaining Compound 5-4 7.7 g (75%).

### Preparation of Compound 5-5

The same method as the preparation of Compound 2-5 was conducted by using Compound 5-4 4.3 g (0.010 mol) in a 500 mL round-bottom flask, thereby obtaining

Compound 5-5 2.0 g (52%).

### Preparation of Compound 5-6

The same method as the preparation of Compound 1-5 was conducted by using 7H-benzo[c]carbazole 8.9 g (41.10 mmol) and 4-(biphenyl-4-yl)-2-chloroquinazoline 15.6 g (49.32 mmol), thereby obtaining Compound 5-6 10.9 g (52%).

### Preparation of Compound 5-7

The same method as the preparation of Compound 1-6 was conducted by using Compound 5-6 15.9 g (35.64 mmol), thereby obtaining Compound 5-7 14.6 g (78%).

### Preparation of Compound 7

The same method as the preparation of Compound 1 was conducted by using Compound 5-5 6.8 g (17.4 mmol) and Compound 5-7 11 g (20.88 mmol), thereby obtaining Compound 75.8 g (7.3 mmol, 42%).

MS/EIMS found 795, calculated 794.96

### Preparation Example 6

### Preparation of Compound 18

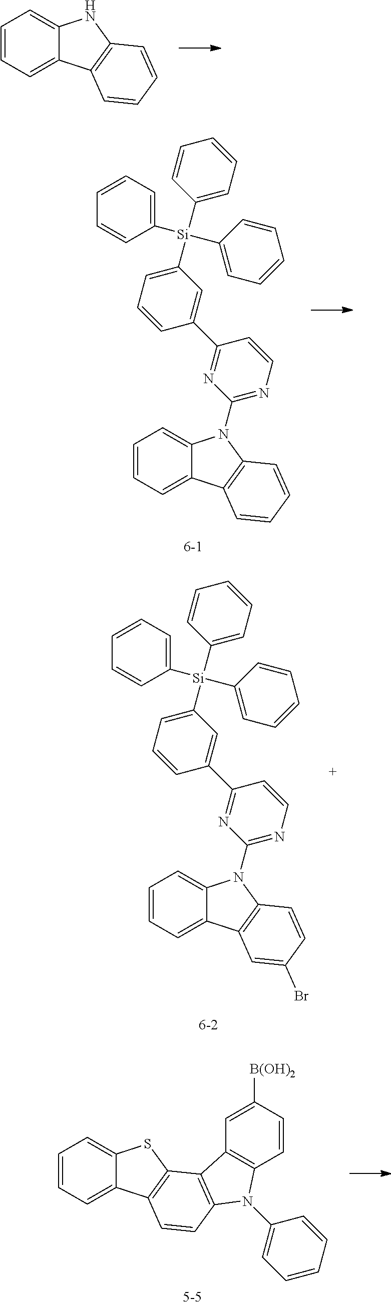 [SMILES: c1ccc2c(c1)[nH]c1ccccc12 ; c1ccc([Si](c2ccccc2)(c2ccccc2)c2cccc(-c3ccnc(-n4c5ccccc5c5ccccc54)n3)c2)cc1 ; Brc1ccc2c(c1)c1ccccc1n2-c1nccc(-c2cccc([Si](c3ccccc3)(c3ccccc3)c3ccccc3)c2)n1 ; OB(O)c1ccc2c(c1)c1c3sc4ccccc4c3ccc1n2-c1ccccc1]

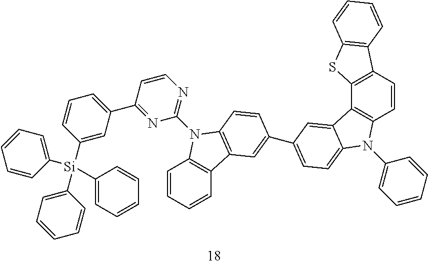 [SMILES: c1ccc(-n2c3ccc(-c4ccc5c(c4)c4ccccc4n5-c4nccc(-c5cccc([Si](c6ccccc6)(c6ccccc6)c6ccccc6)c5)n4)cc3c3c4sc5ccccc5c4ccc32)cc1]

### Preparation of Compound 6-1

The same method as the preparation of Compound 1-5 was conducted by using 7H-benzo[c]carbazole 8.9 g (41.10 mmol) and 2-chloro-4-(3-(triphenylsilyl)phenyl)pyrimidine 22.1 g (49.32 mmol), thereby obtaining Compound 6-1 16.9 g (71%).

### Preparation of Compound 6-2

The same method as the preparation of Compound 1-6 was conducted by using Compound 6-1 20.7 g (35.64 mmol), thereby obtaining Compound 6-2 16.2 g (69%).

### Preparation of Compound 18

The same method as the preparation of Compound 1 was conducted by using Compound 5-5 6.8 g (17.4 mmol) and Compound 6-2 13.8 g (20.88 mmol), thereby obtaining Compound 187.6 g (8.2 mmol, 47%).

MS/EIMS found 928, calculated 927.20

### Preparation Example 7

### Preparation of Compound 30

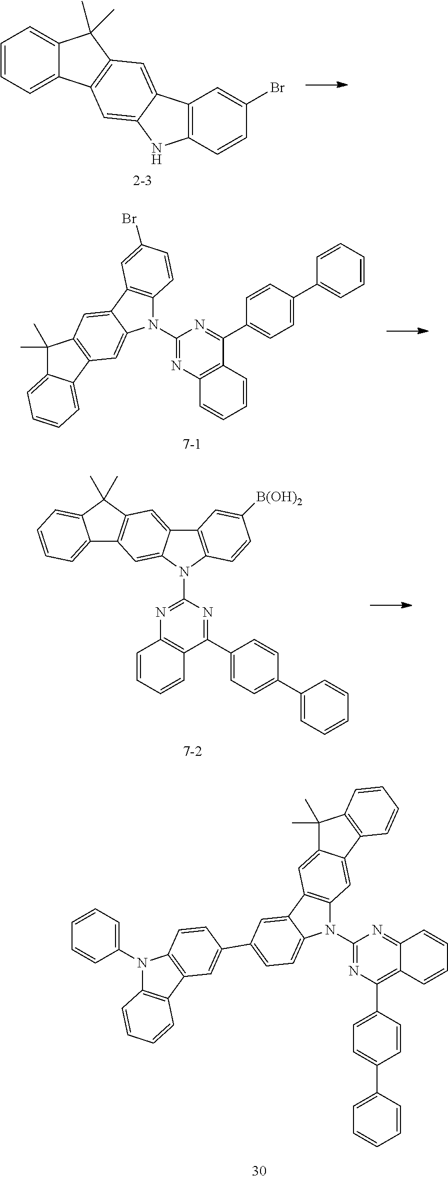 [SMILES: CC1(C)c2ccccc2-c2cc3[nH]c4ccc(Br)cc4c3cc21 ; CC1(C)c2ccccc2-c2cc3c(cc21)c1cc(Br)ccc1n3-c1nc(-c2ccc(-c3ccccc3)cc2)c2ccccc2n1 ; CC1(C)c2ccccc2-c2cc3c(cc21)c1cc(B(O)O)ccc1n3-c1nc(-c2ccc(-c3ccccc3)cc2)c2ccccc2n1 ; CC1(C)c2ccccc2-c2cc3c(cc21)c1cc(-c2ccc4c(c2)c2ccccc2n4-c2ccccc2)ccc1n3-c1nc(-c2ccc(-c3ccccc3)cc2)c2ccccc2n1]

### Preparation of Compound 7-1

Compound 2-3 9 g (0.024 mol) was put into a 3 L 2-neck round-bottom flask, and the same method as the preparation of Compound 2-4 was conducted by using 4-(biphenyl-4-yl)-2-chloroquinazoline 9.2 g (0.029 mol), thereby obtaining Compound 7-1 8.6 g (56%).

### Preparation of Compound 7-2

The same method as the preparation of Compound 2-5 was conducted by using Compound 7-1 6.4 g (0.010 mol) in a 500 mL round-bottom flask, thereby obtaining Compound 7-2 3.2 g (53%).

### Preparation of Compound 30

The same method as the preparation of Compound 1 was conducted by using Compound 7-2 10.6 g (17.4 mmol) and 3-bromo-9-phenyl-9H-carbazole 6.7 g (20.88 mmol), thereby obtaining Compound 309.9 g (12.3 mmol, 71%).

MS/EIMS found 805, calculated 804.98

### Preparation Example 8

### Preparation of Compound 31

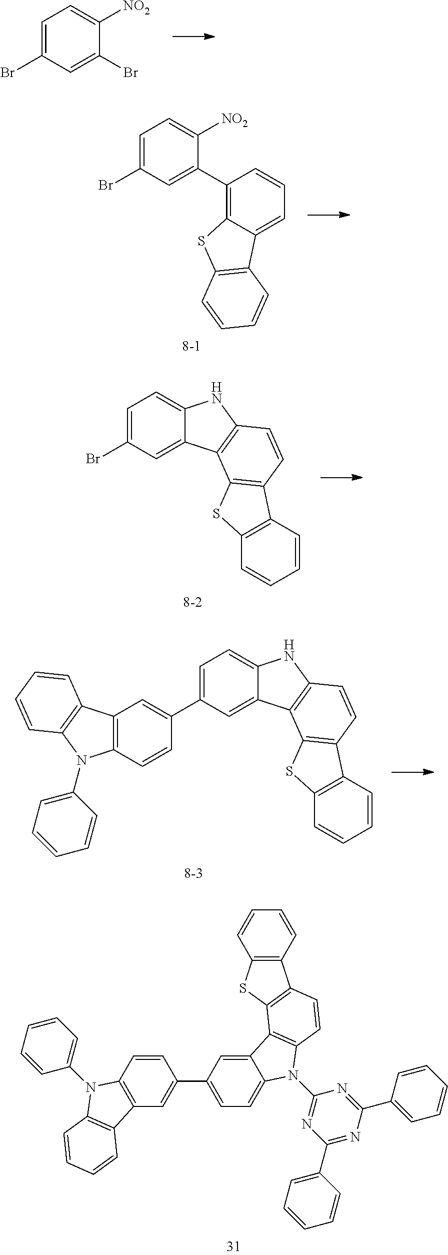 [SMILES: O=[N+]([O-])c1ccc(Br)cc1Br ; O=[N+]([O-])c1ccc(Br)cc1-c1cccc2c1sc1ccccc12 ; Brc1ccc2[nH]c3ccc4c5ccccc5sc4c3c2c1 ; c1ccc(-n2c3ccccc3c3cc(-c4ccc5[nH]c6ccc7c8ccccc8sc7c6c5c4)ccc32)cc1 ; c1ccc(-c2nc(-c3ccccc3)nc(-n3c4ccc(-c5ccc6c(c5)c5ccccc5n6-c5ccccc5)cc4c4c5sc6ccccc6c5ccc43)n2)cc1]

### Synthesis of Compound 8-1

2,4-dibromonitrobenzene (37 g, 131.5 mmol), 4-dibenzothiophenboronic acid (20 g, 87.69 mmol), Pd(PPh₃)₄ (3.0 g, 2.63 mmol), toluene (400 ml), and 1.5M Na₂CO₃ (100 ml) were put, followed by stirring at 100° C. After 4 hours, the resultant material was cooled to room temperature, and then extracted with EA, followed by washing with distilled water. Afterthat, drying over magnesium sulfate and then distillation under reduced pressure were performed, followed by column separation, thereby obtaining Compound 8-1, (16 g, 47.48%).

### Synthesis of Compound 8-2

Compound 8-1 (16 g, 41.64 mmol), triethylphosphite 100 mL, and 1,2-dichlorobenzene 100 mL were put, followed by stirring at 100° C. After 4 hours, the resultant material was cooled to room temperature. Then, distillation under reduced pressure was performed, followed by column separation, thereby obtaining Compound 8-2 (5 g, 34.08%).

### Synthesis of Compound 8-3

Compound 8-2 (5 g, 14.19 mmol), N-phenylcarbazole-3-boronic acid (4.9 g, 17.03 mmol), Pd(PPh₃)₄ (0.82 g, 0.71 mmol), toluene (100 ml), 2M K₂CO₃ (20 ml), and ethanol (20 ml) were put, followed by stirring under reflux. After 8 hours, the resultant material was cooled to room temperature and then extracted with EA. Washing with distilled water and drying over magnesium sulfate were performed. Then, distillation under reduced pressure was performed, followed by column separation, thereby obtaining Compound 8-3 (6 g, 82.16%).

### Synthesis of Compound 31

Compound 8-3 (6 g, 11.65 mmol) and 2-chloro-4,6-diphenyltriazine (3.74 g, 13.99 mmol) were dissolved in DMF, and then NaH (0.69 g, 17.48 mmol) was added thereinto, followed by stirring at room temperature for 15 hours. Methanol and distilled water were put thereinto, and the thus generated solid was filtered. Then, column separation with the respect to the solid was performed, thereby obtaining compound 31 5 g (57.543%).

MS/EIMS found 719.85, calculated 719.20

### Preparation Example 9

### Preparation of Compound 37

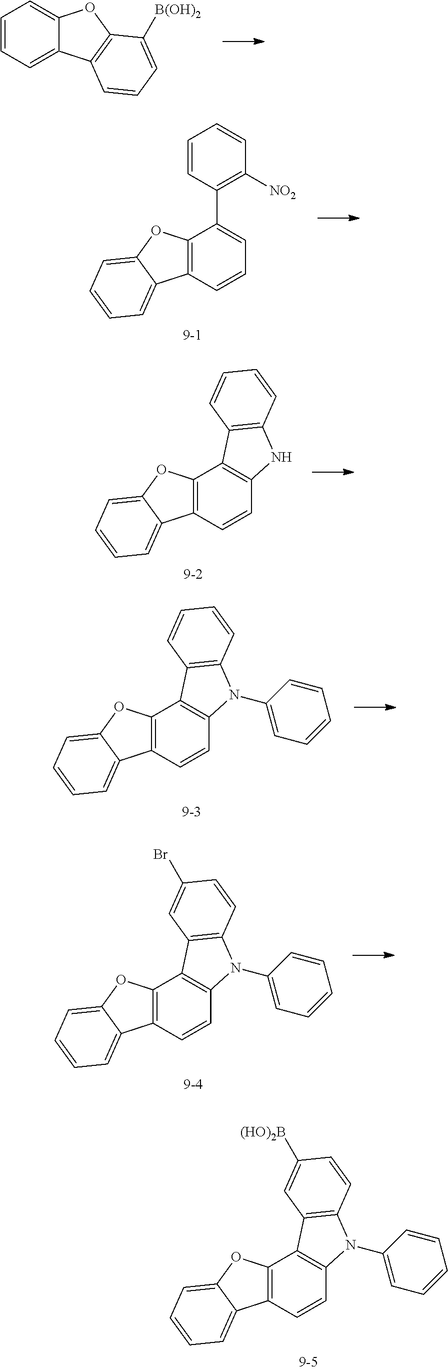 [SMILES: OB(O)c1cccc2c1oc1ccccc12 ; O=[N+]([O-])c1ccccc1-c1cccc2c1oc1ccccc12 ; c1ccc2c(c1)[nH]c1ccc3c4ccccc4oc3c12 ; c1ccc(-n2c3ccccc3c3c4oc5ccccc5c4ccc32)cc1 ; Brc1ccc2c(c1)c1c3oc4ccccc4c3ccc1n2-c1ccccc1 ; OB(O)c1ccc2c(c1)c1c3oc4ccccc4c3ccc1n2-c1ccccc1]

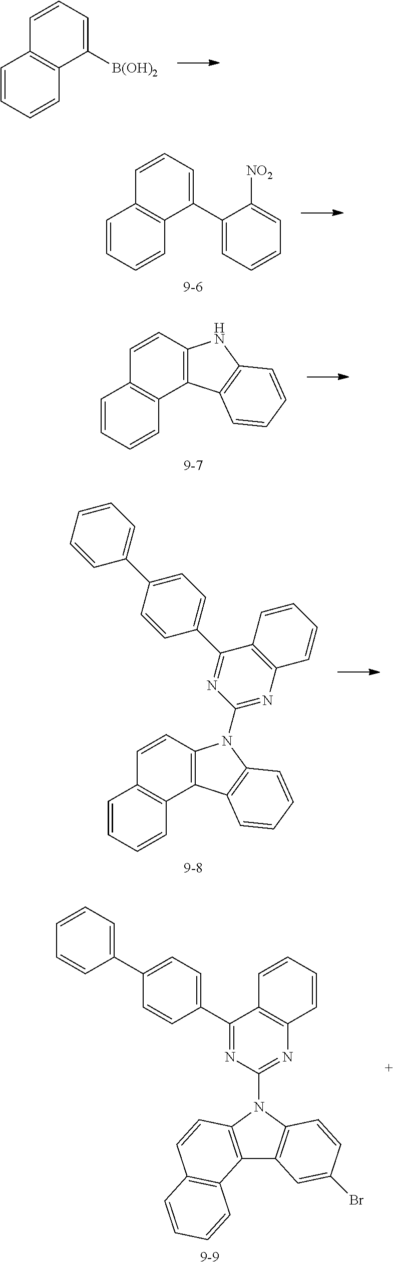 [SMILES: OB(O)c1cccc2ccccc12 ; O=[N+]([O-])c1ccccc1-c1cccc2ccccc12 ; c1ccc2c(c1)ccc1[nH]c3ccccc3c12 ; c1ccc(-c2ccc(-c3nc(-n4c5ccccc5c5c6ccccc6ccc54)nc4ccccc34)cc2)cc1 ; Brc1ccc2c(c1)c1c3ccccc3ccc1n2-c1nc(-c2ccc(-c3ccccc3)cc2)c2ccccc2n1]

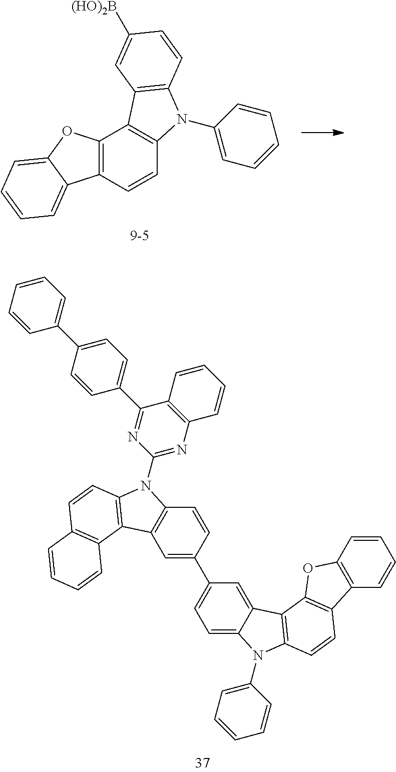 [SMILES: OB(O)c1ccc2c(c1)c1c3oc4ccccc4c3ccc1n2-c1ccccc1 ; c1ccc(-c2ccc(-c3nc(-n4c5ccc(-c6ccc7c(c6)c6c8oc9ccccc9c8ccc6n7-c6ccccc6)cc5c5c6ccccc6ccc54)nc4ccccc34)cc2)cc1]

### Preparation of Compound 9-1

1-bromo-2-nitrobenzene 15 g (0.074 mol) was put into a 1 L 2-neck round-bottom flask, and then, the same method as the preparation of Compound 2-1 was conducted by using dibenzo[b,d]furan-4-ylboronic acid 21.9 g (0.096 mol), thereby obtaining Compound 9-1 18.5 g (82%).

### Preparation of Compound 9-2

The same method as the preparation of Compound 2-2 was conducted by using Compound 9-1 20.8 g (0.068 mol) in a 1 L 2-neck round-bottom flask, thereby obtaining Compound 9-2 6.9 g (37%).

### Preparation of Compound 9-3

The same method as the preparation of Compound 2-3 was conducted by using Compound 9-2 7.7 g (0.028 mol) in a 1 L 2-neck round-bottom flask, thereby obtaining Compound 9-3 7.4 g (76%).

### Preparation of Compound 9-4

The same method as the preparation of Compound 2-4 was conducted by using Compound 9-3 8.4 g (0.024 mol) in a 3 L 2-neck round-bottom flask, thereby obtaining Compound 9-4 7.7 g (75%).

### Preparation of Compound 9-5

The same method as the preparation of Compound 2-5 was conducted by using Compound 9-4 4.3 g (0.010 mol) in a 500 mL round-bottom flask, thereby obtaining Compound 9-5 2.0 g (52%).

### Preparation of Compound 9-6

1-bromo-2-nitrobenzene 15 g (0.074 mol) was put into a 1 L 2-neck round-bottom flask, and then, the same method as the preparation of Compound 2-1 was conducted by using naphthalen-1-ylboronic acid 16.5 g (0.096 mol), thereby obtaining Compound 9-6 16.4 g (89%).

### Preparation of Compound 9-7

The same method as the preparation of Compound 2-2 was conducted by using Compound 9-6 18.9 g (0.076 mol) in a 1 L 2-neck round-bottom flask, thereby obtaining Compound 9-7 11.2 g (68%).

### Preparation of Compound 9-8

The same method as the preparation of Compound 1-5 was conducted by using Compound 9-7 8.9 g (41.10 mmol) and 4-(biphenyl-4-yl)-2-chloroquinazoline 15.6 g (49.32 mmol), thereby obtaining Compound 9-8 10 g (42%).

### Preparation of Compound 9-9

The same method as the preparation of Compound 1-6 was conducted by using Compound 9-8 17.7 g (35.64 mmol), thereby obtaining Compound 9-9 14.2 g (69%).

### Preparation of Compound 37

The same method as the preparation of Compound 1 was conducted by using Compound 8-5 6.6 g (17.4 mmol) and Compound 8-9 12.0 g (20.88 mmol), thereby obtaining Compound 377.3 g (8.8 mmol, 51%).

MS/EIMS found 829, calculated 828.95

### Preparation Example 10

### Preparation of Compound 40

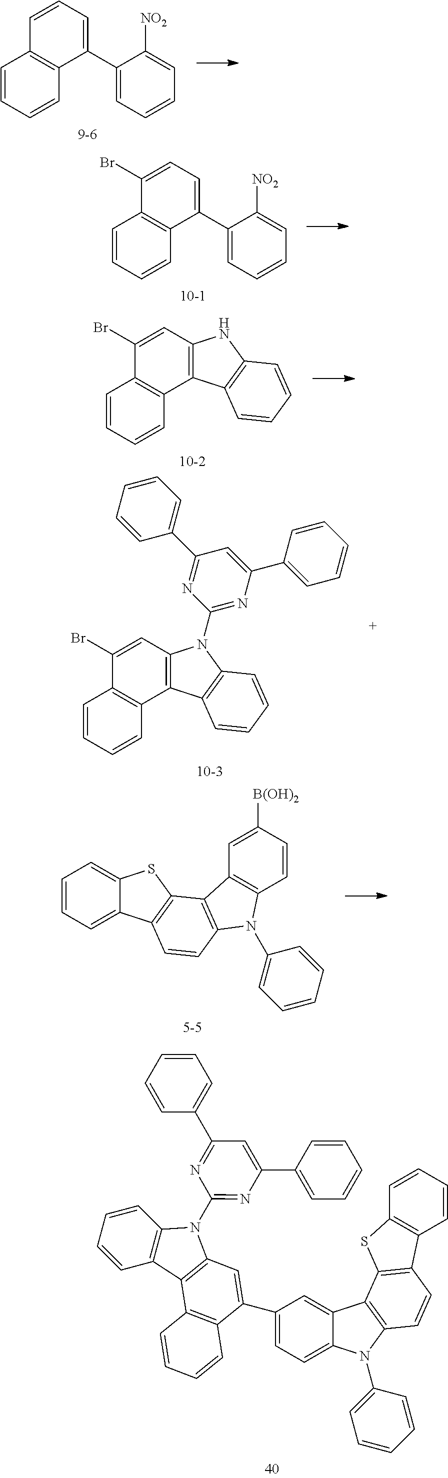

### Preparation of Compound 10-1

Compound 9-6 9.0 g (36.1 mmol) and N-bromosuccinimide 7.6 g (43.3 mmol) were dissolved in dichloromethane 300 mL, followed by stirring at room temperature for 12 hours. Distillation under reduced pressure was performed, and the thus obtained solid was sequentially washed with distilled water, methanol, and then hexane, thereby obtaining Compound 10-1 9.6 g (81.3%).

### Preparation of Compound 10-2

The same method as the preparation of Compound 2-2 was conducted by using Compound 10-124.9 g (0.076 mol) in a 1 L 2-neck round-bottom flask, thereby obtaining Compound 10-211.9 g (52%).

### Preparation of Compound 10-3

The same method as the preparation of Compound 1-5 was conducted by using Compound 10-212.2 g (41.10 mmol) and 2-chloro-4,6-diphenyl-1,3,5-triazine 13.2 g (49.32 mmol), thereby obtaining Compound 10-313.4 g (62%).

### Preparation of Compound 40

The same method as the preparation of Compound 1 was conducted by using Compound 5-5 6.8 g (17.4 mmol) and Compound 10-311 g (20.88 mmol), thereby obtaining Compound 407.3 g (9.2 mmol, 53%).

MS/EIMS found 795, calculated 794.96

### Preparation Example 11

### Preparation of Compound 62

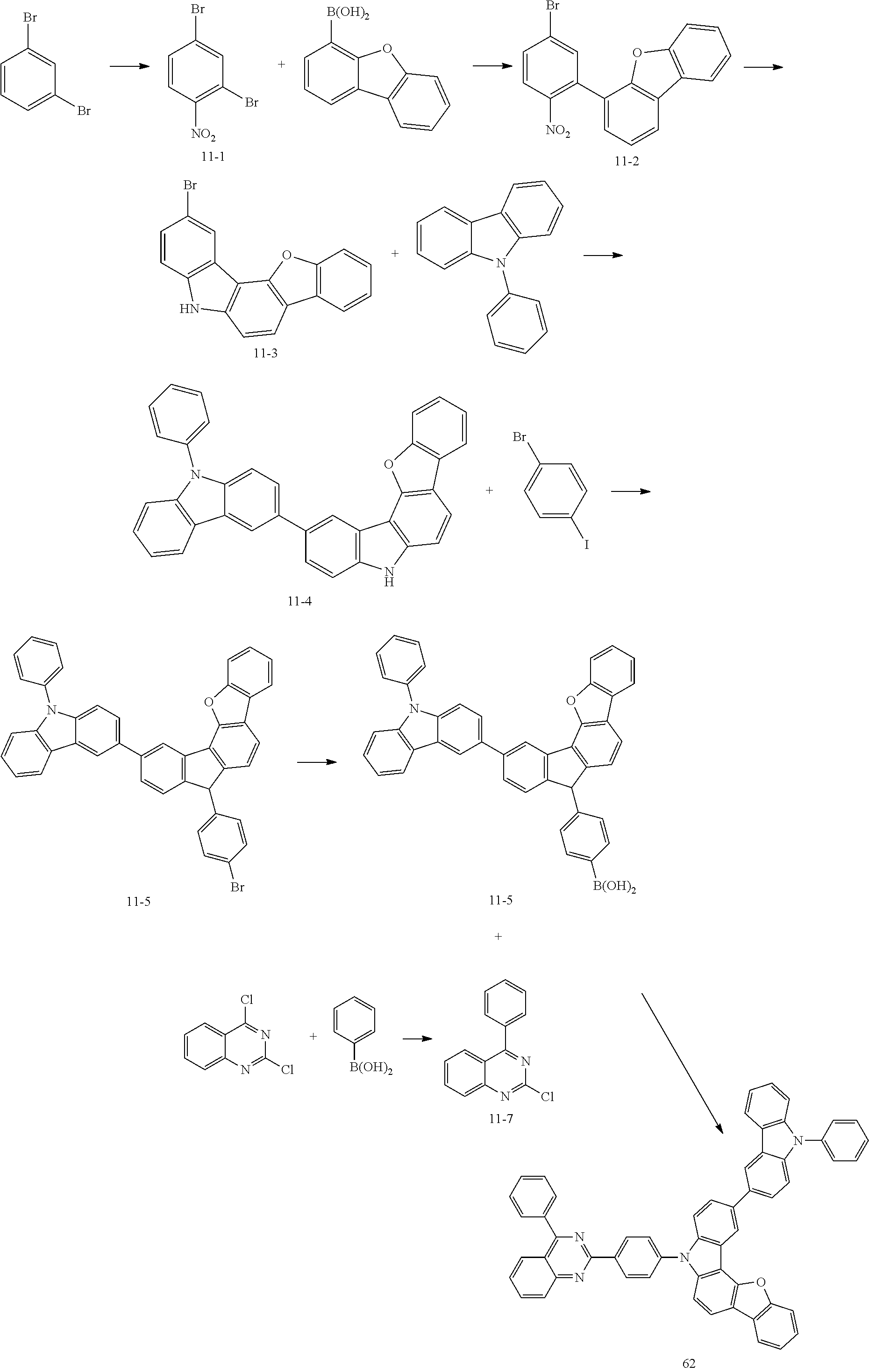

### Preparation of Compound 11-1

Sulfuric acid (124 mL) and 60% nitric acid (28 mL) were mixed, and then cooled to 0° C. Then, 1,3-dibromobenzene (50 g, 0.21 mol) was slowly added dropwise thereto, followed by stirring for 30 minutes. After the stirring was completed, ice water was added thereto, followed by extraction with EA (300 mL). The organic layer was dried over anhydrous magnesium sulfate, followed by distillation under reduced pressure and then silica column purification, thereby obtaining Compound 11-1 (20 g, 34%).

### Preparation of Compound 11-2

Compound 11-1 (30 g, 106.8 mmol), dibenzo[b,d]furan-4-ylboronic acid (22.6 g, 106.8 mmol), Pd(PPh₃)₄ (6.2 g, 5.4 mmol) and K₂CO₃ (34 g, 321 mmol) were put into a mixed solvent of toluene (500 mL)/EtOH (100 mL)/purified water (100 mL), followed by stirring at 80° C. for 2 hours. After the reaction was completed, the resultant material was cooled to room temperature, and then stood. Then, the water layer was removed and the organic layer was concentrated, followed by silica column purification, thereby obtaining Compound 11-2 (20 g, 51%).

### Preparation of Compound 11-3

Compound 11-2 (20 g, 54.3 mmol) was dissolved in 1,2-dichlorobenzene (100 mL), and P(OEt)₃ (100 mL) was added thereto, followed by stirring at 150° C. for 20 hours. The resultant mixture was cooled to room temperature, and then 1,2-dichlororbenzene and P(OEt)₃, which are the solvent, were removed by distillation under reduced pressure. Afterthat, extraction with EA (500 mL) and distilled water (50 mL) were performed, and then the organic layer was dried over anhydrous magnesium sulfate, and distilled under reduced pressure, followed by silica column purification, thereby obtaining Compound 11-3 (12 g, 66%).

### Preparation of Compound 11-4

Compound 11-2 (6 g, 17.85 mmol), 9-phenyl-9H-carbazol-3-ylboronic acid (6.2 g, 21.6 mmol), Pd(PPh₃)₄ (1 g, 0.87 mmol) and K₂CO₃ (7.4 g, 53.5 mmol) were put into a mixed solution of toluene (100 mL)/EtOH (20 mL)/purified water (20 mL), followed by stirring at 120° C. for 3 hours. After the reaction was completed, the resultant material was cooled to room temperature, and then stood. Then, the water layer was removed and the organic layer was concentrated, followed by silica column purification, thereby obtaining Compound 11-4 (6.8 g, 76%).

### Preparation of Compound 11-5

Compound 11-4 (4.6 g, 9.23 mmol), 1-bromo-4-iodobenzene (5.22 g, 18.45 mmol), CuI (880 mg, 4.62 mmol), 1,2-diaminoethane (1.24 mL, 18.45 mmol) and Cs₂CO₃(9.02 g, 27.7 mmol) were put into toluene (100 mL), followed by stirring at 120° C. for 20 hours. After the reaction was completed, the resultant material was cooled to room temperature, and then stood. Then, the water layer was removed and the organic layer was concentrated, followed by silica column purification, thereby obtaining Compound 11-5 (5.6 g, 93%).

### Preparation of Compound 11-6

Compound 11-5 (5.6 g, 8.57 mmol) was dissolved in THF (50 mL), and n-BuLi (4.1 ml, 2.5M in hexane) was slowly added thereto at −78° C. The mixture was stirred at the same temperature for 1 hour, and then triisoproxyborane (3 mL) was added thereto, followed by stirring at room temperature for 2 hours. Upon completion of the stirring, the reaction was stopped by using an aqueous ammonium chloride solution 20 mL, followed by washing with distilled water and extraction with EA were performed. Then, the organic layer was dried over anhydrous magnesium sulfate, followed by distillation under reduced pressure and recrystallization, thereby obtaining Compound 11-6 (2.6 g, 49%).

### Preparation of Compound 11-7

2,4-dichloroquinazoline (16 g, 80.4 mmol), phenylboronic acid (11.8 g, 96.5 mmol), Na₂CO₃ (25.56 g, 241 mmol) and Pd(PPh₃)₄ (4.6 g, 4 mmol) were put in a mixed solution of toluene (100 mL)/EtOH (20 mL)/purified water (20 mL), followed by stirring at 80° C. for 15 hours. After the reaction was completed, the resultant material was cooled to room temperature, and then stood. Then, the water layer was removed and the organic layer was concentrated, followed by silica column purification, thereby obtaining Compound 11-7 (14 g, 72%).

### Preparation of Compound 62

Compound 11-6 (9.28 g, 15 mmol), Compound 11-7 (3 g, 12.46 mmol), Pd(PPh₃)₄ (716 mg, 0.62 mmol) and K₂CO₃ (5.2 g, 37.4 mmol) were put in a mixed solution of toluene (50 mL)/EtOH (10 mL)/purified water (10 mL), followed by stirring at 120° C. for 5 hours. After the reaction was completed, the resultant material was cooled to room temperature, and then stood. Then, the water layer was removed and the organic layer was concentrated, followed by silica column purification, thereby obtaining Compound 62 (6.1 g, 63%).

MS/EIMS found 778.90, calculated 778.27

### Preparation Example 12

### Preparation of Compound 70

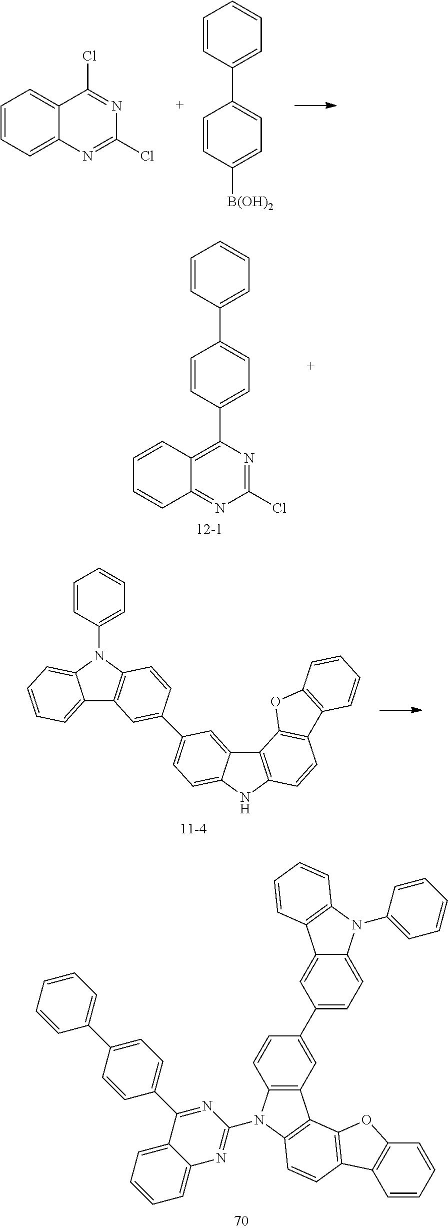

### Preparation of Compound 12-1

2,4-dichloroquinazoline (5 g, 25.1 mmol), biphenyl-4-ylboronic acid (5.4 g, 27.3 mmol), Na₂CO₃ (8 g, 75.3 mmol) and Pd(PPh₃)₄ (1.45 g, 1.26 mmol) were put in a mixed solution of toluene (120 mL)/EtOH (30 mL)/purified water (30 mL), followed by stirring at 120° C. for 15 hours. After the reaction was completed, the resultant material was cooled to room temperature, and then stood. Then, the water layer was removed and the organic layer was concentrated, followed by silica column purification, thereby obtaining Compound 12-1 (6.2 g, 78%).

### Preparation of Compound 70

Compound 11-4 (2.3 g, 4.6 mmol) and Compound 12-1 (1.75 g, 5.5 mmol) were suspended in DMF (33 mL), and then 60% NaH (221 mg, 5.5 mmol) was put thereinto at room temperature, followed by stirring for 12 hours. After the stirring was completed, purified water (500 mL) was put thereinto, followed by filtering under reduced pressure. The thus obtained solid was subjected to sequential trituration with MeOH/EA, DMF, and EA/THF, and then dissolved in MC, followed by silica filtering and then trituration with MeOH/EA, thereby obtaining Compound 70 (1.7 g, 47%).

MS/EIMS found 778.90, calculated 778.27

### Example 1

### Manufacture of OLED device Using Compound for Organic Electronic Material According to the Present Invention

An OLED device was manufactured by using a luminescent material of the present invention. First, a transparent electrode ITO thin film (15Ω/□) obtained from glass for an OLED (manufactured by Samsung-Corning Inc.) was subjected to ultrasonic washing with trichloroethylene, acetone, ethanol, and distilled water, sequentially, and stored in isopropanol before use. Next, an ITO substrate was installed on a substrate holder of a vacuum vapor deposition apparatus, and [4,4′,4″-tris(N,N-(2-naphthyl)-phenylamino)triphenylamine] was put in a cell of the vacuum vapor deposition apparatus, which was then evacuated until a vacuum degree in the chamber reached 10⁻⁶ torr. Then, electric current was applied to the cell to perform varporization, thereby forming a hole injection layer having a thickness of 60 nm on the ITO substrate. Then, N,N′-di(4-biphenyl)-N,N′-di(4-biphenyl)-4,4′-diaminobiphenyl was put in another cell of the vacuum vapor deposition apparatus, and electric current was applied to the cell to perform vaporization, thereby forming a hole transport layer having a thickness of 20 nm on the hole injection layer. After forming the hole injection layer and the hole transport layer, a luminescent layer was formed thereon as follows. Compound 31 as a host material was put in a cell of the vacuum vapor deposition apparatus, and D-16 as a dopant was put in another cell thereof, and then the two materials were vaporized at different rates to perform doping of 10 wt %, thereby depositing a luminescent layer having a thickness of 30 nm on the hole transport layer. Then, a luminescent layer having a thickness of 30 nm was deposited on the hole transport layer. Then, Alq[tris(8-hydroxyquinoline)-aluminum(III)] was deposited on the luminescent layer in a thickness of 20 nm, as an electron transport layer. Then, Lig (lithium quinolate) was deposited in a thickness of 1 to 2 nm, as an electron injection layer, and then an Al cathode was formed to have a thickness of 150 nm by using another vacuum vapor deposition apparatus, thereby manufacturing an OLED device. Respective compounds according to the materials were purified by vacuum sublimation under 10-6 torr, and used as luminescent materials for an OLED.

As a result, a current of 7.24 mA/cm² flowed at a voltage of 5.6 V, and green light emission of 2600 cd/m² was confirmed.

### Example 2

### Manufacture of OLED Device Using Compound for Organic Electronic Material According to the Present Invention

An OLED device was manufactured by the same method as Example 1 except that Compound 51 as a luminescent material was used for a host. As a result, a current of 2.25 mA/cm² flowed at a voltage of 4.8 V, and green light emission of 930 cd/m² was confirmed.

### Example 3

### Manufacture of OLED Device Using Compound for Organic Electronic Material According to the Present Invention

An OLED device was manufactured by the same method as Example 1 except that Compound 52 as a luminescent material was used for a host.

As a result, a current of 4.76 mA/cm² flowed at a voltage of 5.3 V, and green light emission of 1820 cd/m² was confirmed.

### Comparative Example

### Manufacture of OLED Device Using Luminescent of the Related Art

An OLED device was manufactured by the same method as Example 1 except that 4,4′-N,N′-dicarbazole-biphenyl as a luminescent material was used for a host to form a luminescent layer, aluminum(III)bis(2-methyl-8-quinolinato)4-phenylphenolate as a hole blocking layer was deposited on the luminescent layer in a thickness of 10 nm.

As a result, a current of 9.52 mA/cm² flowed at a voltage of 7.2 V, and green light emission of 3000 cd/m² was confirmed.

### Example 4

### Manufacture of OLED Device Using Compound for Organic Electronic Material According to the Present Invention

An OLED device was manufactured by using a luminescent material of the present invention. First, a transparent electrode ITO thin film (15Ω/□) obtained from glass for an OLED (manufactured by Samsung-Corning Inc.) was subjected to ultrasonic washing with trichloroethylene, acetone, ethanol, and distilled water, sequentially, and stored in isopropanol before use. Next, an ITO substrate was installed on a substrate holder of a vacuum vapor deposition apparatus, and N1,N1′-([1,1′-biphenyl]-4,4′-diyl)bis(N1-(naphthalen-1-yl)-N4,N4-diphenylbenzene-1,4-diamine was put in a cell of the vacuum vapor deposition apparatus, which was then evacuated until vacuum degree in the chamber reached 10-6 torr. Then, electric current was applied to the cell to perform vaporization, thereby forming a hole injection layer having a thickness of 60 nm on the ITO substrate. Then, N,N′-di(4-biphenyl)-N,N′-di(4-biphenyl)-4,4′-diaminobiphenyl was put in another cell of the vacuum vapor deposition apparatus, and electric current was applied to the cell to perform vaporization, thereby forming a hole transport layer having a thickness of 20 nm on the hole injection layer. After forming the hole injection layer and the hole transport layer, a luminescent layer was formed thereon as follows. Compound 70 as a host material was put in a cell of the vacuum vapor deposition apparatus, and D-7 as a dopant was put in another cell thereof, and then the two materials were vaporized at different rates to perform doping of 4 wt %, thereby depositing a luminescent layer having a thickness of 30 nm on the hole transport layer. Subsequently, 2-(4-(9,10-di(naphthalen-2-yl)anthracen-2-yl)phenyl)-1-phenyl-1H-benzo[d]imidazole was put in a cell of the vacuum deposition apparatus, and Lithium quinolate was put in another cell of the vacuum deposition apparatus, and then the two materials were vaporized at different rates to perform doping of 50 wt %, thereby forming an electron transport layer of 30 nm on the luminescent layer. Next, lithium quinolate was deposited in a thickness of 2 nm, as an electron injection layer, and then an Al cathode was deposited in a thickness of 150 nm by using another vacuum deposition apparatus, thereby manufacturing an OLED device. Respective compounds according to the materials were purified by vacuum sublimation under 10⁻⁶ torr, and used as luminescent materials for an OLED.

As a result, a current of 15.1 mA/cm² flowed at a voltage of 4.8V, and red light emission of 1800 cd/m² was confirmed.

### Example 5

### Manufacture of OLED Device Using Compound for Organic Electronic Material According to the Present Invention

An OLED device was manufactured by the same method as Example 1 except that, a luminescent material, Compound 62 was used for a host and Compound D-7 was used for a dopant.

As a result, a current of 6.72 mA/cm² flowed at a voltage of 3.3 V, and red light emission of 820 cd/m² was confirmed.

### Example 6

### Manufacture of OLED Device Using Compound for Organic Electronic Material According to the Present Invention

An OLED device was manufactured by the same method as Example 1 except that, a luminescent material, Compound 61 was used for a host and Compound D-7 was used for a dopant.

As a result, a current of 13.2 mA/cm² flowed at a voltage of 4.5 V, and red light emission of 1320 cd/m² was confirmed.

### Comparative Example

### Manufacture of OLED Device Using Luminescent of the Related Art

An OLED device was manufactured by the same method as Example 1 except that a luminescent material, 4,4′-N,N′-dicarbazole-biphenyl was used for a host and Compound D-11 was used for a dopant to form a luminescent layer, aluminum(III)bis(2-methyl-8-quinolinato)4-phenylphenolate as a hole blocking layer was deposited on the luminescent layer in a thickness of 10 nm.

As a result, a current of 54.4 mA/cm² flowed at a voltage of 9.4 V, and red light emission of 2300 cd/m² was confirmed.

It could be confirmed that the compound for an organic electronic material developed by the present invention had superior excellent luminous properties as compared with the material of the related art. In addition, a device using the compound for an organic electronic material according to the present invention as a host material has excellent luminous properties, and further, can induce an increase in power efficiency by lowering a driving voltage, thereby improving power consumption.

1. A compound for an organic electronic material expressed by Chemical Formula 1 below: 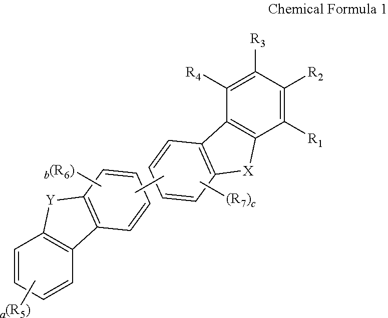
wherein Chemical Formula 1, X represents —O—, —S—, —CR₁₁R₁₂— or N-L₁-Ar₁; Y represents —O—, —S—, —CR₁₃R₁₄— or N-L₂-Ar₂; but Y necessarily represents N-L₁-Ar₁ when X represents —O—, —S— or —CR₁₁R₁₂—; X necessarily represents N-L₂-Ar₂ when Y represents —O—, —S— or —CR₁₃R₁₄—; one of R₁ through R₄ is linked to an adjacent substituent via 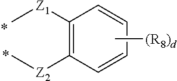
to form a fused ring, the others thereof independently represent hydrogen, deuterium, halogen, substituted or unsubstituted (C1-C30)alkyl, substituted or unsubstituted (C6-C30)aryl, substituted or unsubstituted (C3-C30)heteroaryl, substituted or unsubstituted (C3-C30)cycloalkyl, substituted or unsubstituted 5- to 7-membered heterocycloalkyl, substituted or unsubstituted (C6-C30)ar(C1-C30)alkyl, substituted or unsubstituted (C6-C30)aryl fused with one or more cycloalkyl, 5- to 7-membered heterocycloalkyl fused with one or more substituted or unsubstituted aromatic rings, (C3-C30)cycloalkyl fused with one or more substituted or unsubstituted aromatic rings, —NR₂₁R₂₂, —SiR₂₃R₂₄R₂₅, —SR₂₆, —OR₂₇, (C2-C30)alkenyl, (C2-C30)alkynyl, cyano, nitro, or hydroxy; L₁ and L₂ independently represent a single bond, substituted or unsubstituted (C6-C30)arylene, or substituted or unsubstituted (C3-C30)heteroarylene; Ar₁ and Ar₂ independently represent hydrogen, deuterium, halogen, substituted or unsubstituted (C1-C30)alkyl, substituted or unsubstituted (C6-C30)aryl, or substituted or unsubstituted (C3-C30)heteroaryl; one of Z₁ and Z₂ represents a single bond, and the other thereof represents —O—, —S—, —CR₃₁R₃₂—, —SiR₃₃R₃₄—, or —NR₃₅—; R₅ through R₅ independently represent hydrogen, deuterium, halogen, substituted or unsubstituted (C1-C30)alkyl, substituted or unsubstituted (C6-C30)aryl, substituted or unsubstituted (C3-C30)heteroaryl, substituted or unsubstituted (C3-C30)cycloalkyl, substituted or unsubstituted 5- to 7-membered heterocycloalkyl, substituted or unsubstituted (C6-C30)ar(C1-C30)alkyl, substituted or unsubstituted (C6-C30)aryl fused with one or more (C3-C30)cycloalkyl, 5- to 7-membered heterocycloalkyl fused with one or more substituted or unsubstituted aromatic rings, (C3-C30)cycloalkyl fused with one or more substituted or unsubstituted aromatic rings, —NR₂₁R₂₂, —SiR₂₃R₂₄R₂₅, —SR₂₆, —OR₂₇, (C2-C30)alkenyl, (C2-C30)alkynyl, cyano, nitro, or hydroxy; R₁₁ through R₁₄, R₂₁ through R₂₇, and R₃₁ through R₃₅ independently represent hydrogen, deuterium, halogen, substituted or unsubstituted (C1-C30)alkyl, substituted or unsubstituted (C6-C30)aryl, substituted or unsubstituted (C2-C30)heteroaryl, substituted or unsubstituted (C3-C30)cycloalkyl, substituted or unsubstituted 5- to 7-membered heterocycloalkyl, substituted or unsubstituted (C6-C30)ar(C1-C30)alkyl, substituted or unsubstituted (C6-C30)aryl fused with one or more (C3-C30)cycloalkyl, 5- to 7-membered heterocycloalkyl fused with one or more substituted or unsubstituted aromatic rings, or (C3-C30)cycloalkyl fused with one or more substituted or unsubstituted aromatic rings; a through d independently represent an integer of 1 to 4, and they may be the same or different from one another when they independently represent an integer of 2 or more; b and c independently represent an integer of 1 to 3, and they may be the same or different from each other when they independently represent an integer of 2 or more; and the heterocycloalkyl, heteroarylene, and heteroaryl each include one or more hetero atoms selected from B, N, O, S, P(═O), Si and P.

2. The compound of claim 1, wherein substituents further substituted at L₁, L₂, Ar₁, Ar₂, Z₁, Z₂, R₁ through R₈, R₁₁ through R₁₄, R₂₁ through R₂₇ and R₃₁ through R₃₅ independently represent one or more selected from the group consisting of deuterium, halogen, (C1-C30)alkyl, (C1-C30)alkyl substituted or unsubstituted with halogen, (C6-C30)aryl, (C2-C30)heteroaryl, (C2-C30)heteroaryl substituted or unsubstituted with (C1-C30)alkyl, (C2-C30)heteroaryl substituted or unsubstituted with (C6-C30)aryl, (C3-C30)cycloalkyl, 5- to 7-membered heterocycloalkyl, tri(C1-C30)alkylsilyl, tri(C1-C30)arylsilyl, di(C1-C30)alkyl(C6-C30)arylsilyl, (C1-C30)alkyldi(C6-C30)arylsilyl, (C2-C30)alkenyl, (C2-C30)alkynyl, cyano, carbazolyl, di(C1-C30)alkylamino, di(C6-C30)arylamino, (C1-C30)alkyl(C6-C30)arylamino, di(C6-C30)arylboronyl, di(C1-C30)alkylboronyl, (C1-C30)alkyl(C6-C30)arylboronyl, (C6-C30)ar(C1-C30)alkyl, (C1-C30)alkyl(C6-C30)aryl, carboxyl, nitro, and hydroxy.

3. The compound of claim 1, wherein the 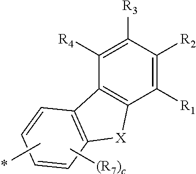
is selected from the structures below: 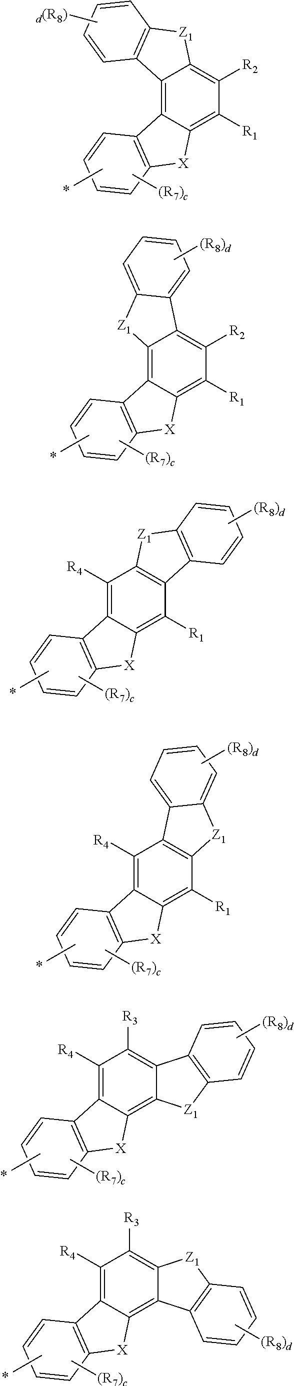 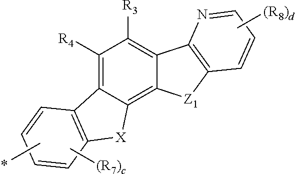
Wherein, X represents —O—, —S—, —CR₁₁R₁₂— or N-L₁-Ar₁; Z₁ represents —O—, —S—, —CR₃₁R₃₂—, —SiR₃₃R₃₄— or —NR₃₅—; R₁ through R₄ independently represent hydrogen, deuterium, halogen, substituted or unsubstituted (C1-C30)alkyl, substituted or unsubstituted (C6-C30)aryl, substituted or unsubstituted (C3-C30)heteroaryl, substituted or unsubstituted (C3-C30)cycloalkyl, substituted or unsubstituted 5- to 7-membered heterocycloalkyl, substituted or unsubstituted (C6-C30)ar(C1-C30)alkyl, substituted or unsubstituted (C6-C30)aryl fused with one or more (C3-C30)cycloalkyl, 5- to 7-membered heterocycloalkyl fused with one or more substituted or unsubstituted aromatic rings, (C3-C30)cycloalkyl fused with one or more substituted or unsubstituted aromatic rings, —NR₂₁R₂₂, —SiR₂₃R₂₄R₂₅, —SR₂₆, —OR₂₇, (C2-C30)alkenyl, (C2-C30)alkynyl, cyano, nitro, or hydroxy; and R₇, R₈, R₂₁ through R₂₇, R₃₁ through R₃₅, and c and d are the same as defined in claim 1.

4. The compound of claim 1, wherein the 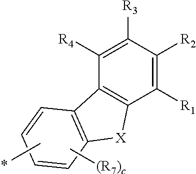
represents 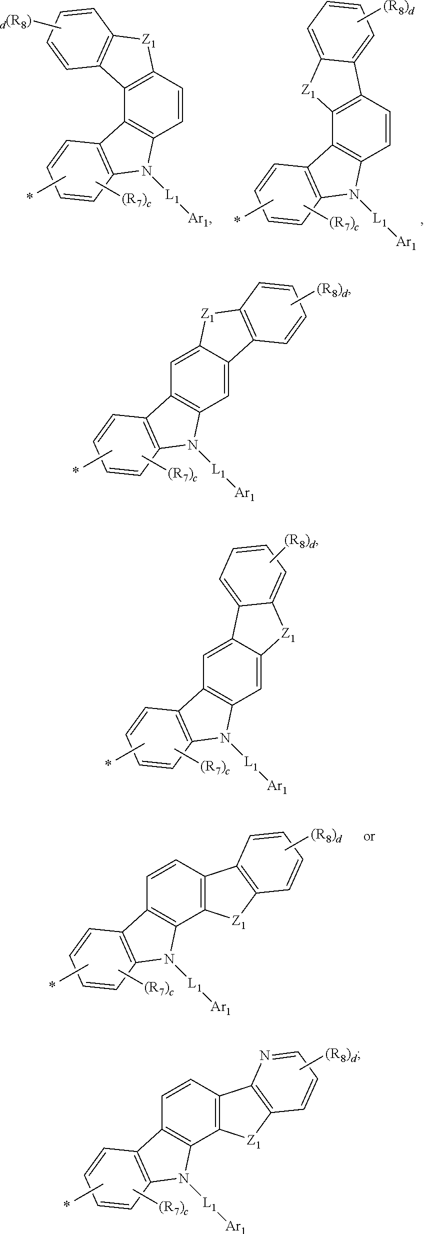
Y represents —O—, —S—, —CR₁₃R₁₄— or N-L₂-Ar₂; Z₁ represents —O—, —S—, —CR₃₁R₃₂—, —SiR₃₃R₃₄— or —NR₃₅—, L₁ and L₂ independently represent a single bond, (C6-C30)arylene, or (C3-C30)heteroarylene; Ar₁ and Ar₂ independently represent hydrogen, deuterium, halogen, (C1-C30)alkyl, (C6-C30)aryl, or (C3-C30)heteroaryl; R₅ through R₈ independently represent hydrogen, deuterium, halogen, (C1-C30)alkyl, (C6-C30)aryl, or (C3-C30)heteroaryl; R₁₃, R₁₄, R₃₁ through R₃₅ independently represent hydrogen, deuterium, (C1-C30)alkyl, (C6-C30)aryl, or (C3-C30)heteroaryl; the arylene and heteroarylene of L₁ and L₂, the alkyl, aryl, or heteroaryl of R₅ through R₈, the alkyl, aryl, or heteroaryl of Ar₁ and Ar₂, R₁₃, R₁₄, R₃₁ through R₃₅ independently may be further substituted from one or more selected from the group consisting of deuterium, halogen, (C1-C30)alkyl, (C1-C30)alkyl substituted with halogen, (C6-C30)aryl, (C3-C30)heteroaryl, (C3-C30)heteroaryl substituted with (C6-C30)aryl, (C3-C30)cycloalkyl, N-carbazolyl, (C6-C30)ar(C1-C30)alkyl, and (C1-C30)alkyl(C6-C30)aryl.

5. The compound of claim 1, wherein the compound for an organic electronic material is selected from the compounds below:

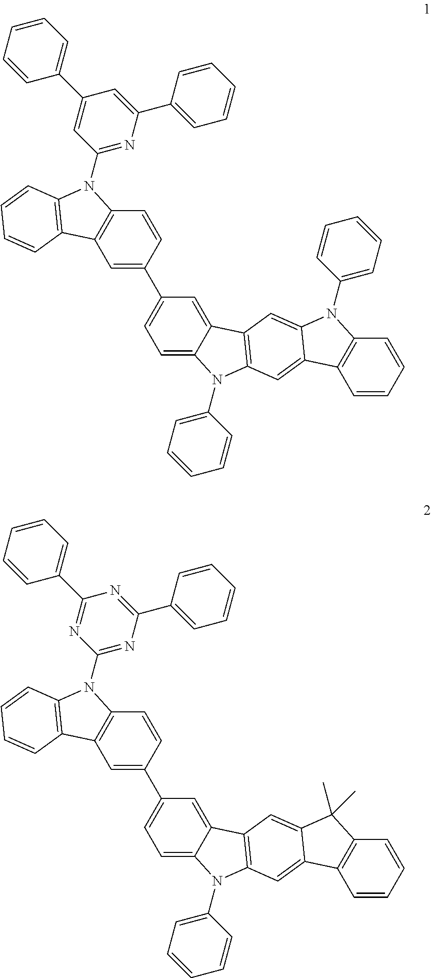 [SMILES: c1ccc(-c2cc(-c3ccccc3)nc(-n3c4ccccc4c4cc(-c5ccc6c(c5)c5cc7c(cc5n6-c5ccccc5)c5ccccc5n7-c5ccccc5)ccc43)c2)cc1 ; CC1(C)c2ccccc2-c2cc3c(cc21)c1cc(-c2ccc4c(c2)c2ccccc2n4-c2nc(-c4ccccc4)nc(-c4ccccc4)n2)ccc1n3-c1ccccc1]

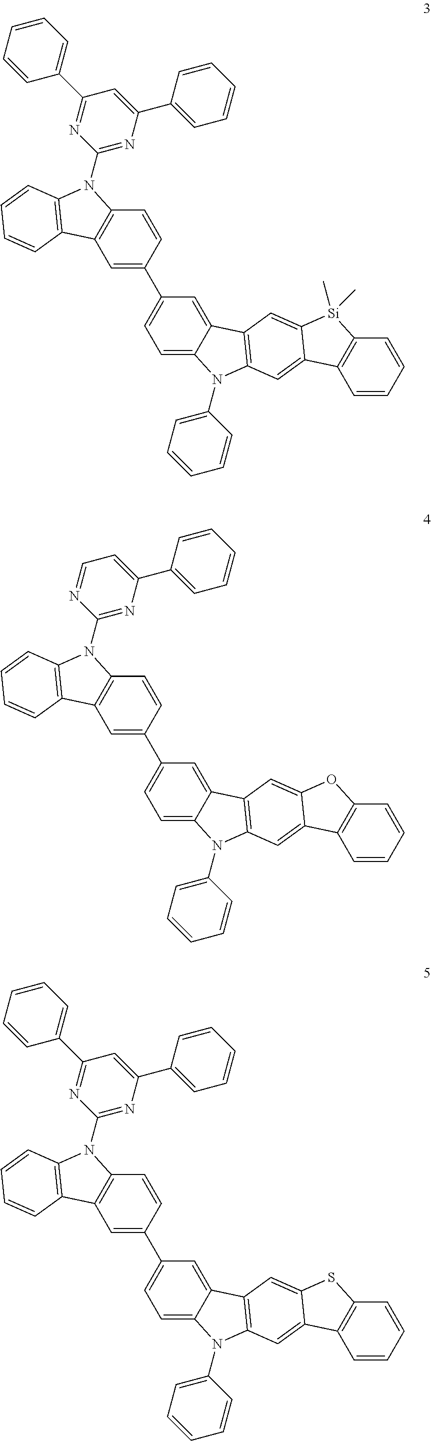 [SMILES: C[Si]1(C)c2ccccc2-c2cc3c(cc21)c1cc(-c2ccc4c(c2)c2ccccc2n4-c2nc(-c4ccccc4)cc(-c4ccccc4)n2)ccc1n3-c1ccccc1 ; c1ccc(-c2ccnc(-n3c4ccccc4c4cc(-c5ccc6c(c5)c5cc7oc8ccccc8c7cc5n6-c5ccccc5)ccc43)n2)cc1 ; c1ccc(-c2cc(-c3ccccc3)nc(-n3c4ccccc4c4cc(-c5ccc6c(c5)c5cc7sc8ccccc8c7cc5n6-c5ccccc5)ccc43)n2)cc1]

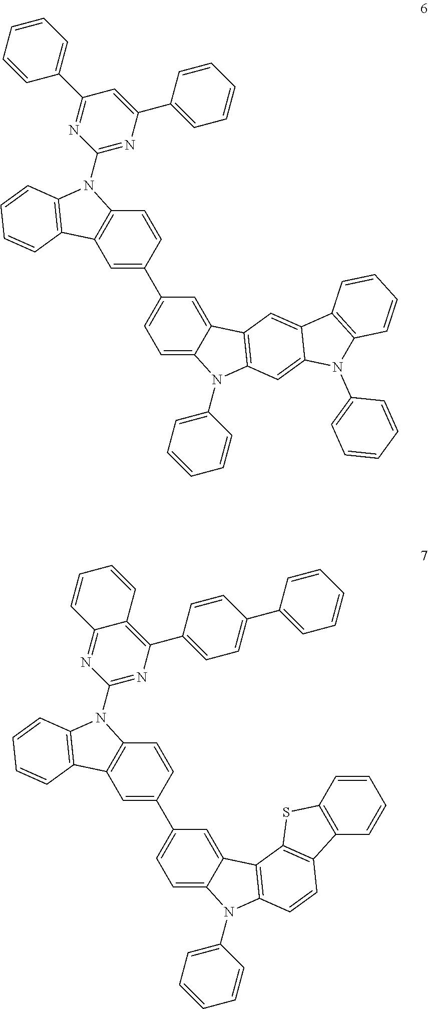 [SMILES: c1ccc(-c2cc(-c3ccccc3)nc(-n3c4ccccc4c4cc(-c5ccc6c(c5)c5cc7c8ccccc8n(-c8ccccc8)c7cc5n6-c5ccccc5)ccc43)n2)cc1 ; c1ccc(-c2ccc(-c3nc(-n4c5ccccc5c5cc(-c6ccc7c(c6)c6c8sc9ccccc9c8ccc6n7-c6ccccc6)ccc54)nc4ccccc34)cc2)cc1]

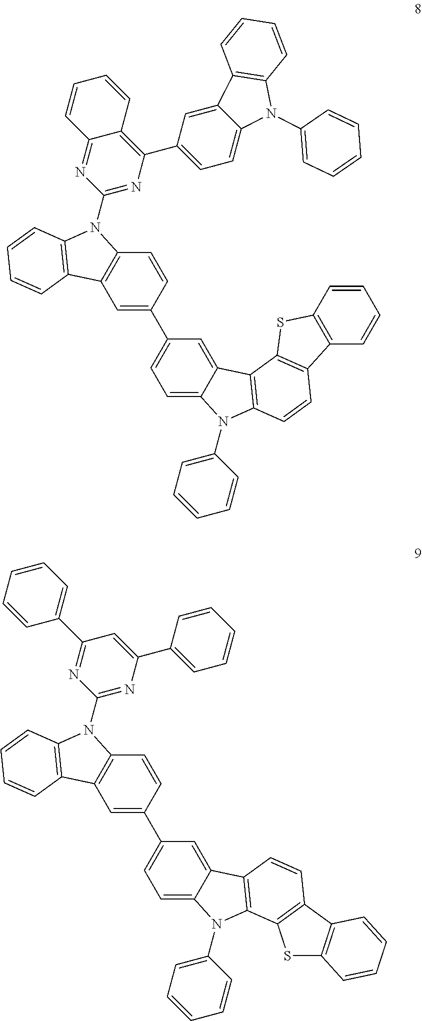 [SMILES: c1ccc(-n2c3ccccc3c3cc(-c4nc(-n5c6ccccc6c6cc(-c7ccc8c(c7)c7c9sc%10ccccc%10c9ccc7n8-c7ccccc7)ccc65)nc5ccccc45)ccc32)cc1 ; c1ccc(-c2cc(-c3ccccc3)nc(-n3c4ccccc4c4cc(-c5ccc6c(c5)c5ccc7c8ccccc8sc7c5n6-c5ccccc5)ccc43)n2)cc1]

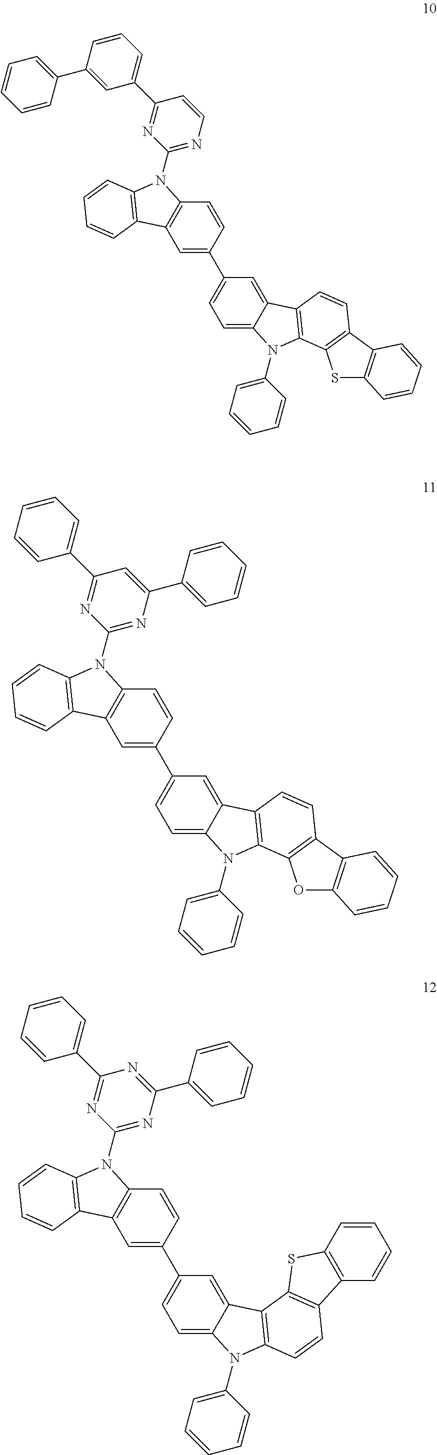 [SMILES: c1ccc(-c2cccc(-c3ccnc(-n4c5ccccc5c5cc(-c6ccc7c(c6)c6ccc8c9ccccc9sc8c6n7-c6ccccc6)ccc54)n3)c2)cc1 ; c1ccc(-c2cc(-c3ccccc3)nc(-n3c4ccccc4c4cc(-c5ccc6c(c5)c5ccc7c8ccccc8oc7c5n6-c5ccccc5)ccc43)n2)cc1 ; c1ccc(-c2nc(-c3ccccc3)nc(-n3c4ccccc4c4cc(-c5ccc6c(c5)c5c7sc8ccccc8c7ccc5n6-c5ccccc5)ccc43)n2)cc1]

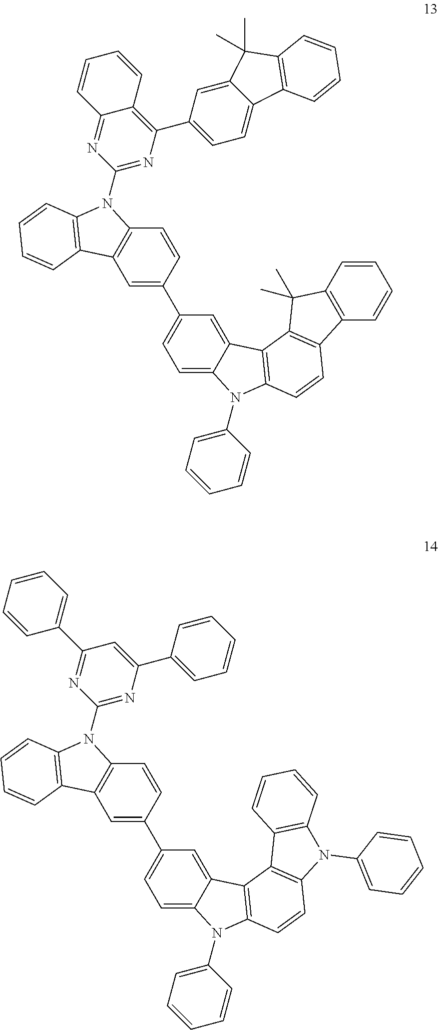 [SMILES: CC1(C)c2ccccc2-c2ccc(-c3nc(-n4c5ccccc5c5cc(-c6ccc7c(c6)c6c8c(ccc6n7-c6ccccc6)-c6ccccc6C8(C)C)ccc54)nc4ccccc34)cc21 ; c1ccc(-c2cc(-c3ccccc3)nc(-n3c4ccccc4c4cc(-c5ccc6c(c5)c5c7c8ccccc8n(-c8ccccc8)c7ccc5n6-c5ccccc5)ccc43)n2)cc1]

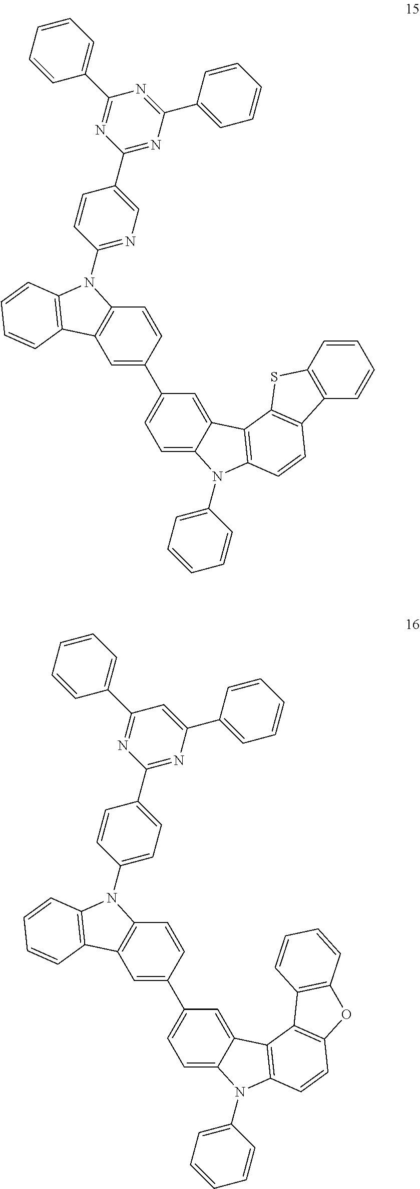 [SMILES: c1ccc(-c2nc(-c3ccccc3)nc(-c3ccc(-n4c5ccccc5c5cc(-c6ccc7c(c6)c6c8sc9ccccc9c8ccc6n7-c6ccccc6)ccc54)nc3)n2)cc1 ; c1ccc(-c2cc(-c3ccccc3)nc(-c3ccc(-n4c5ccccc5c5cc(-c6ccc7c(c6)c6c8c(ccc6n7-c6ccccc6)oc6ccccc68)ccc54)cc3)n2)cc1]

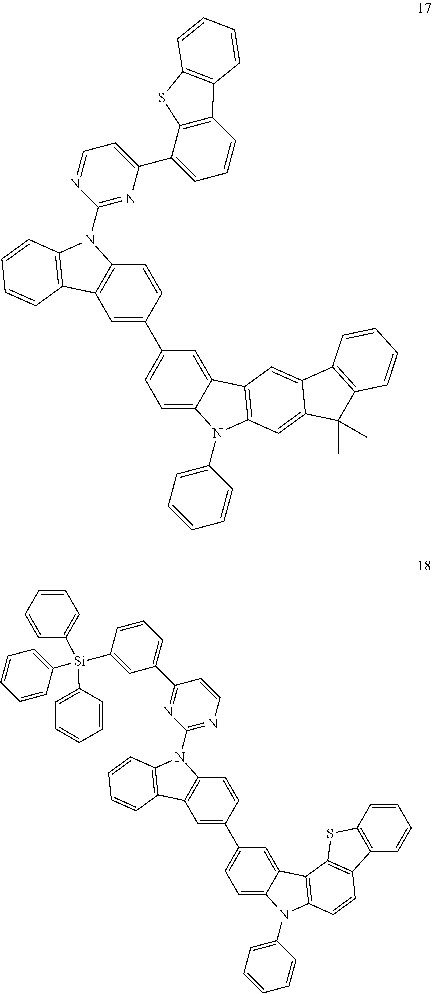 [SMILES: CC1(C)c2ccccc2-c2cc3c4cc(-c5ccc6c(c5)c5ccccc5n6-c5nccc(-c6cccc7c6sc6ccccc67)n5)ccc4n(-c4ccccc4)c3cc21 ; c1ccc(-n2c3ccc(-c4ccc5c(c4)c4ccccc4n5-c4nccc(-c5cccc([Si](c6ccccc6)(c6ccccc6)c6ccccc6)c5)n4)cc3c3c4sc5ccccc5c4ccc32)cc1]

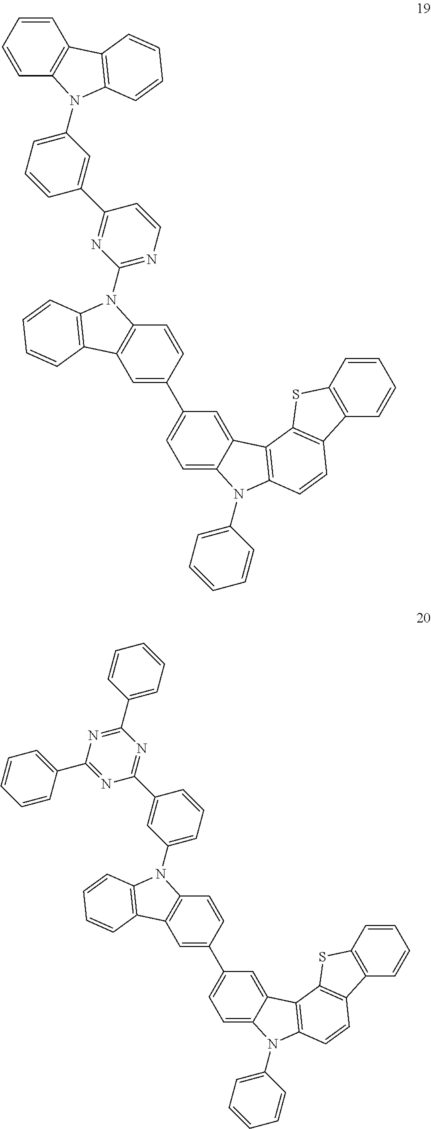 [SMILES: c1ccc(-n2c3ccc(-c4ccc5c(c4)c4ccccc4n5-c4nccc(-c5cccc(-n6c7ccccc7c7ccccc76)c5)n4)cc3c3c4sc5ccccc5c4ccc32)cc1 ; c1ccc(-c2nc(-c3ccccc3)nc(-c3cccc(-n4c5ccccc5c5cc(-c6ccc7c(c6)c6c8sc9ccccc9c8ccc6n7-c6ccccc6)ccc54)c3)n2)cc1]

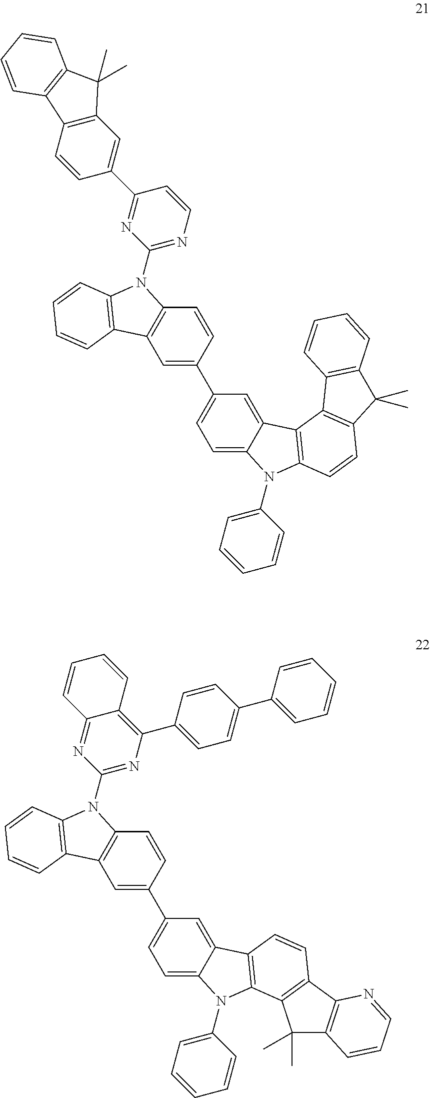 [SMILES: CC1(C)c2ccccc2-c2ccc(-c3ccnc(-n4c5ccccc5c5cc(-c6ccc7c(c6)c6c8c(ccc6n7-c6ccccc6)C(C)(C)c6ccccc6-8)ccc54)n3)cc21 ; CC1(C)c2cccnc2-c2ccc3c4cc(-c5ccc6c(c5)c5ccccc5n6-c5nc(-c6ccc(-c7ccccc7)cc6)c6ccccc6n5)ccc4n(-c4ccccc4)c3c21]

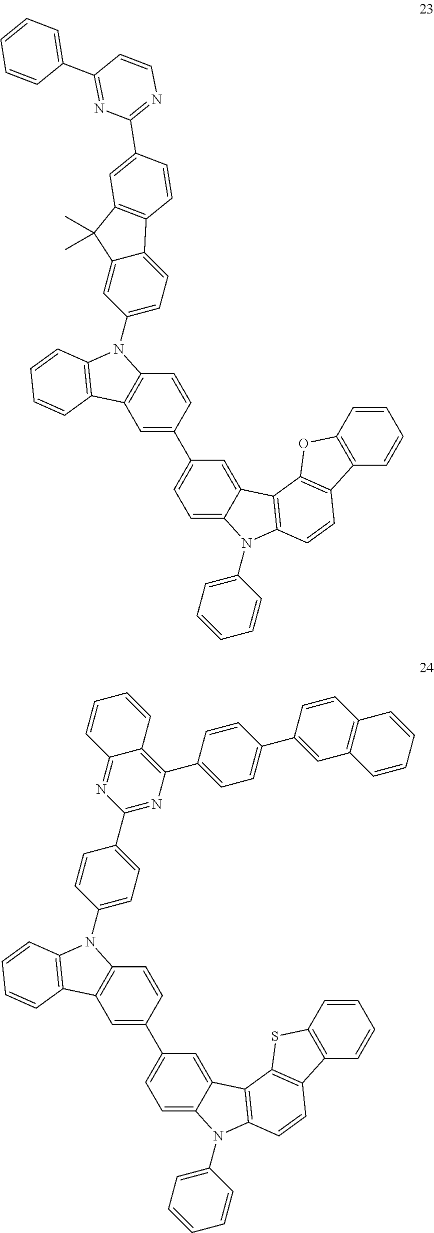 [SMILES: CC1(C)c2cc(-c3nccc(-c4ccccc4)n3)ccc2-c2ccc(-n3c4ccccc4c4cc(-c5ccc6c(c5)c5c7oc8ccccc8c7ccc5n6-c5ccccc5)ccc43)cc21 ; c1ccc(-n2c3ccc(-c4ccc5c(c4)c4ccccc4n5-c4ccc(-c5nc(-c6ccc(-c7ccc8ccccc8c7)cc6)c6ccccc6n5)cc4)cc3c3c4sc5ccccc5c4ccc32)cc1]

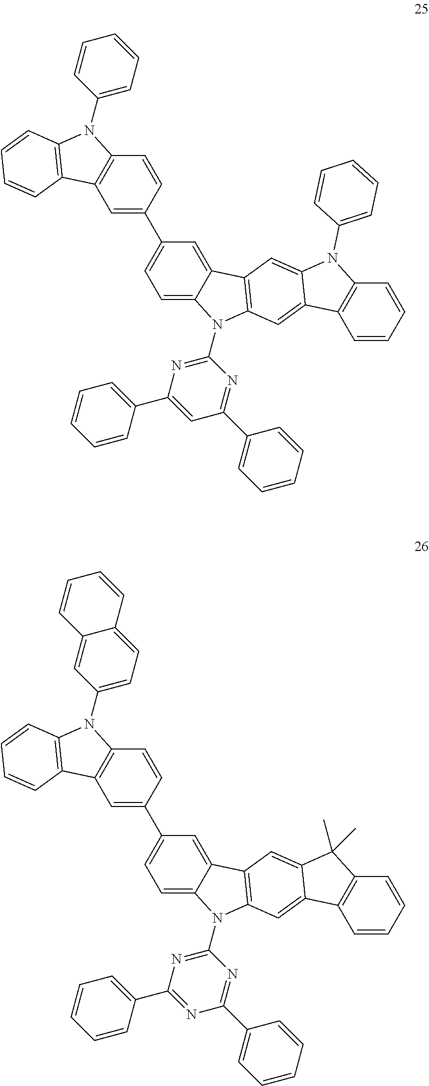 [SMILES: c1ccc(-c2cc(-c3ccccc3)nc(-n3c4ccc(-c5ccc6c(c5)c5ccccc5n6-c5ccccc5)cc4c4cc5c(cc43)c3ccccc3n5-c3ccccc3)n2)cc1 ; CC1(C)c2ccccc2-c2cc3c(cc21)c1cc(-c2ccc4c(c2)c2ccccc2n4-c2ccc4ccccc4c2)ccc1n3-c1nc(-c2ccccc2)nc(-c2ccccc2)n1]

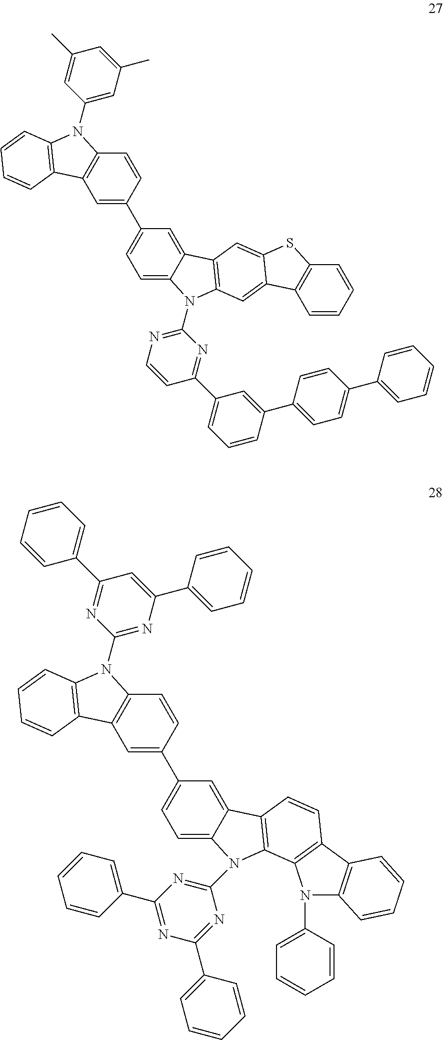 [SMILES: Cc1cc(C)cc(-n2c3ccccc3c3cc(-c4ccc5c(c4)c4cc6sc7ccccc7c6cc4n5-c4nccc(-c5cccc(-c6ccc(-c7ccccc7)cc6)c5)n4)ccc32)c1 ; c1ccc(-c2cc(-c3ccccc3)nc(-n3c4ccccc4c4cc(-c5ccc6c(c5)c5ccc7c8ccccc8n(-c8ccccc8)c7c5n6-c5nc(-c6ccccc6)nc(-c6ccccc6)n5)ccc43)n2)cc1]

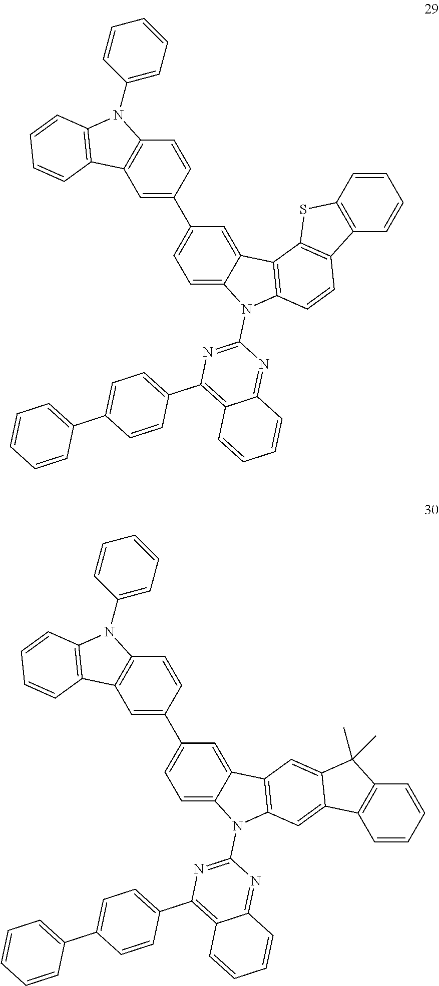 [SMILES: c1ccc(-c2ccc(-c3nc(-n4c5ccc(-c6ccc7c(c6)c6ccccc6n7-c6ccccc6)cc5c5c6sc7ccccc7c6ccc54)nc4ccccc34)cc2)cc1 ; CC1(C)c2ccccc2-c2cc3c(cc21)c1cc(-c2ccc4c(c2)c2ccccc2n4-c2ccccc2)ccc1n3-c1nc(-c2ccc(-c3ccccc3)cc2)c2ccccc2n1]

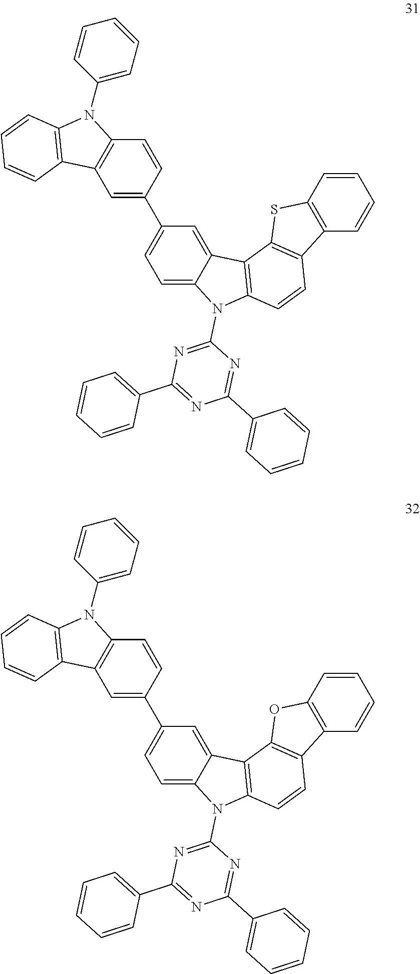 [SMILES: c1ccc(-c2nc(-c3ccccc3)nc(-n3c4ccc(-c5ccc6c(c5)c5ccccc5n6-c5ccccc5)cc4c4c5sc6ccccc6c5ccc43)n2)cc1 ; c1ccc(-c2nc(-c3ccccc3)nc(-n3c4ccc(-c5ccc6c(c5)c5ccccc5n6-c5ccccc5)cc4c4c5oc6ccccc6c5ccc43)n2)cc1]

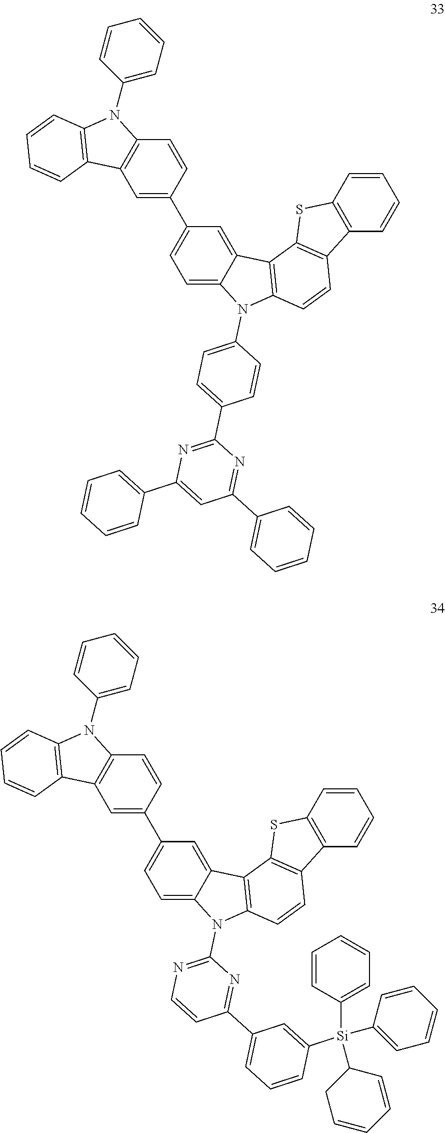 [SMILES: c1ccc(-c2cc(-c3ccccc3)nc(-c3ccc(-n4c5ccc(-c6ccc7c(c6)c6ccccc6n7-c6ccccc6)cc5c5c6sc7ccccc7c6ccc54)cc3)n2)cc1 ; C1=CCC([Si](c2ccccc2)(c2ccccc2)c2cccc(-c3ccnc(-n4c5ccc(-c6ccc7c(c6)c6ccccc6n7-c6ccccc6)cc5c5c6sc7ccccc7c6ccc54)n3)c2)C=C1]

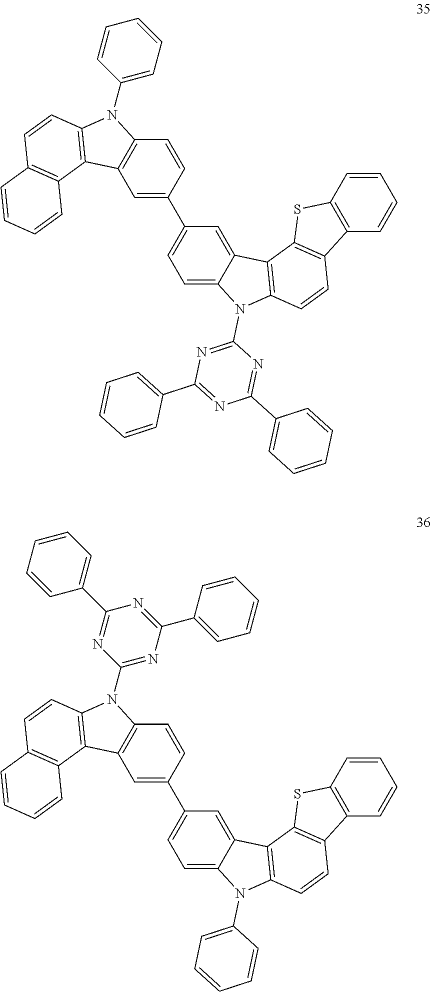 [SMILES: c1ccc(-c2nc(-c3ccccc3)nc(-n3c4ccc(-c5ccc6c(c5)c5c7ccccc7ccc5n6-c5ccccc5)cc4c4c5sc6ccccc6c5ccc43)n2)cc1 ; c1ccc(-c2nc(-c3ccccc3)nc(-n3c4ccc(-c5ccc6c(c5)c5c7sc8ccccc8c7ccc5n6-c5ccccc5)cc4c4c5ccccc5ccc43)n2)cc1]

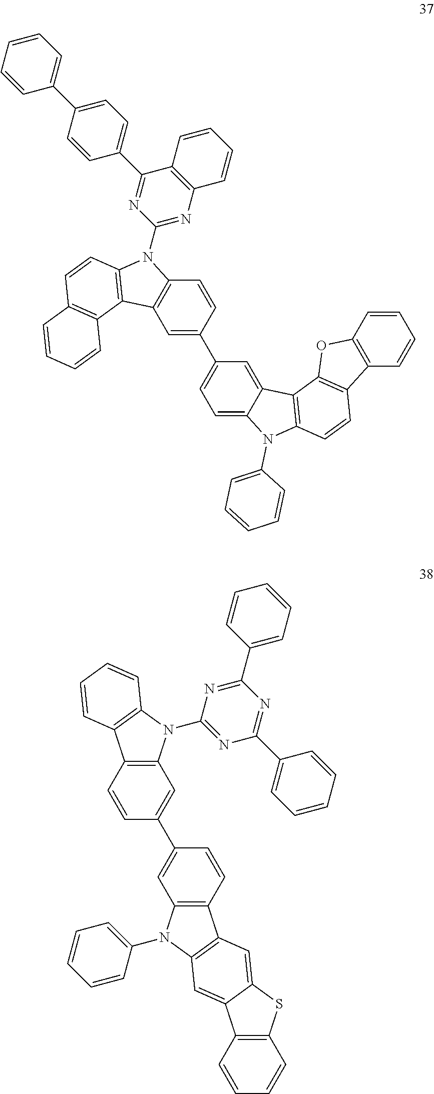 [SMILES: c1ccc(-c2ccc(-c3nc(-n4c5ccc(-c6ccc7c(c6)c6c8oc9ccccc9c8ccc6n7-c6ccccc6)cc5c5c6ccccc6ccc54)nc4ccccc34)cc2)cc1 ; c1ccc(-c2nc(-c3ccccc3)nc(-n3c4ccccc4c4ccc(-c5ccc6c7cc8sc9ccccc9c8cc7n(-c7ccccc7)c6c5)cc43)n2)cc1]

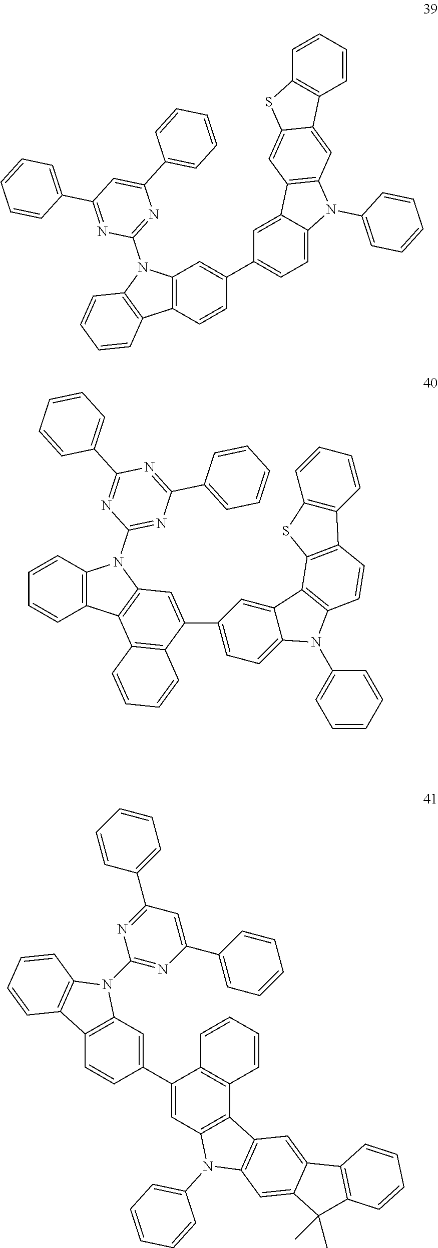

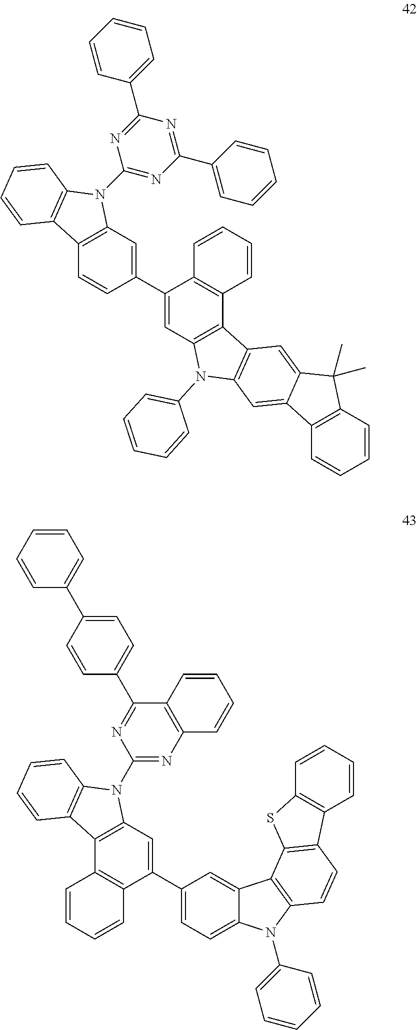 [SMILES: CC1(C)c2ccccc2-c2cc3c(cc21)c1c2ccccc2c(-c2ccc4c5ccccc5n(-c5nc(-c6ccccc6)nc(-c6ccccc6)n5)c4c2)cc1n3-c1ccccc1 ; c1ccc(-c2ccc(-c3nc(-n4c5ccccc5c5c6ccccc6c(-c6ccc7c(c6)c6c8sc9ccccc9c8ccc6n7-c6ccccc6)cc54)nc4ccccc34)cc2)cc1]

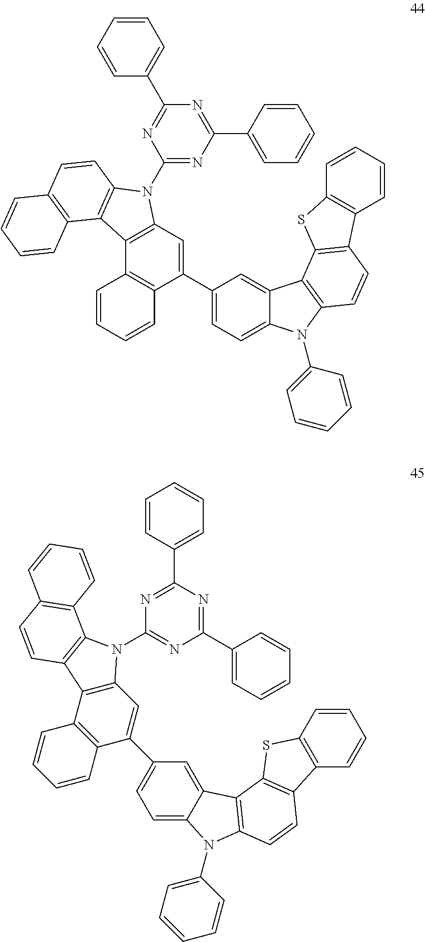 [SMILES: c1ccc(-c2nc(-c3ccccc3)nc(-n3c4ccc5ccccc5c4c4c5ccccc5c(-c5ccc6c(c5)c5c7sc8ccccc8c7ccc5n6-c5ccccc5)cc43)n2)cc1 ; c1ccc(-c2nc(-c3ccccc3)nc(-n3c4cc(-c5ccc6c(c5)c5c7sc8ccccc8c7ccc5n6-c5ccccc5)c5ccccc5c4c4ccc5ccccc5c43)n2)cc1]

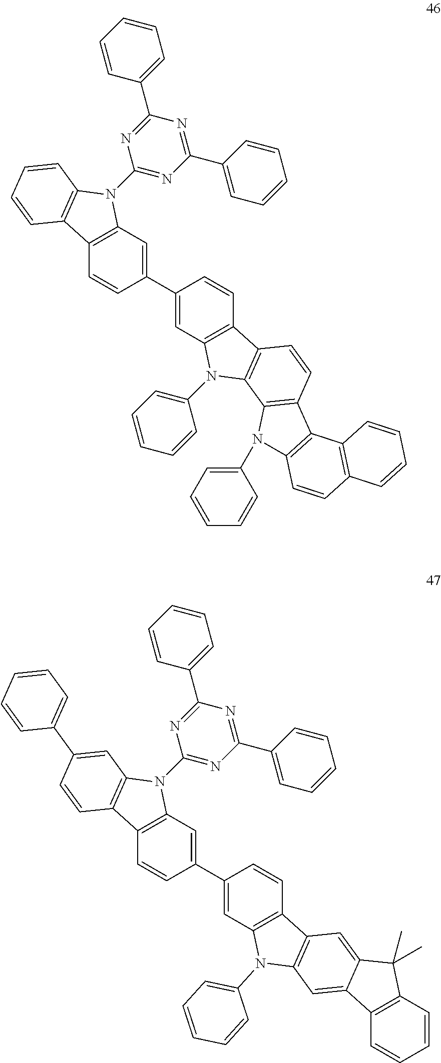 [SMILES: c1ccc(-c2nc(-c3ccccc3)nc(-n3c4ccccc4c4ccc(-c5ccc6c7ccc8c9c%10ccccc%10ccc9n(-c9ccccc9)c8c7n(-c7ccccc7)c6c5)cc43)n2)cc1 ; CC1(C)c2ccccc2-c2cc3c(cc21)c1ccc(-c2ccc4c5ccc(-c6ccccc6)cc5n(-c5nc(-c6ccccc6)nc(-c6ccccc6)n5)c4c2)cc1n3-c1ccccc1]

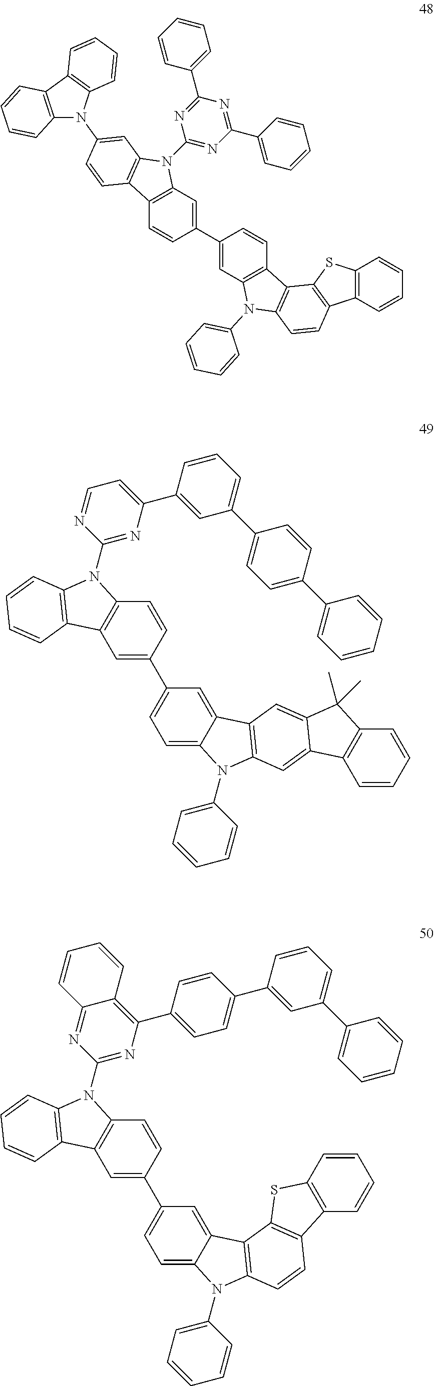 [SMILES: c1ccc(-c2nc(-c3ccccc3)nc(-n3c4cc(-c5ccc6c7c8sc9ccccc9c8ccc7n(-c7ccccc7)c6c5)ccc4c4ccc(-n5c6ccccc6c6ccccc65)cc43)n2)cc1 ; CC1(C)c2ccccc2-c2cc3c(cc21)c1cc(-c2ccc4c(c2)c2ccccc2n4-c2nccc(-c4cccc(-c5ccc(-c6ccccc6)cc5)c4)n2)ccc1n3-c1ccccc1 ; c1ccc(-c2cccc(-c3ccc(-c4nc(-n5c6ccccc6c6cc(-c7ccc8c(c7)c7c9sc%10ccccc%10c9ccc7n8-c7ccccc7)ccc65)nc5ccccc45)cc3)c2)cc1]

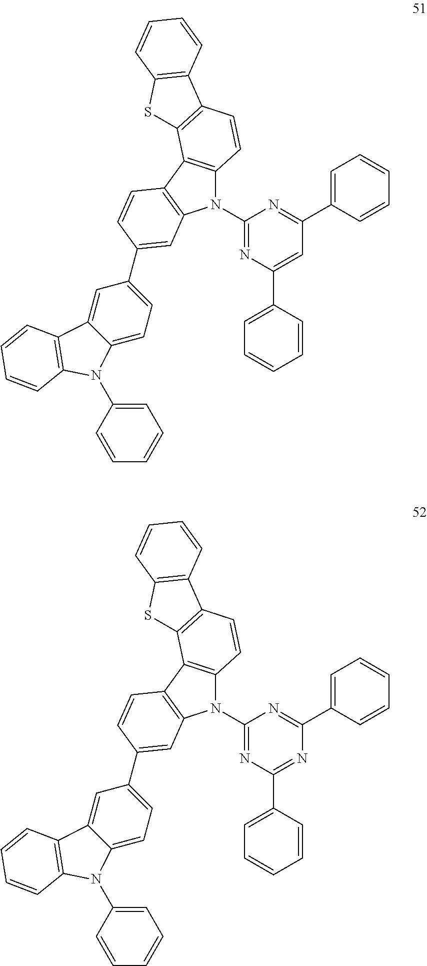 [SMILES: c1ccc(-c2cc(-c3ccccc3)nc(-n3c4cc(-c5ccc6c(c5)c5ccccc5n6-c5ccccc5)ccc4c4c5sc6ccccc6c5ccc43)n2)cc1 ; c1ccc(-c2nc(-c3ccccc3)nc(-n3c4cc(-c5ccc6c(c5)c5ccccc5n6-c5ccccc5)ccc4c4c5sc6ccccc6c5ccc43)n2)cc1]

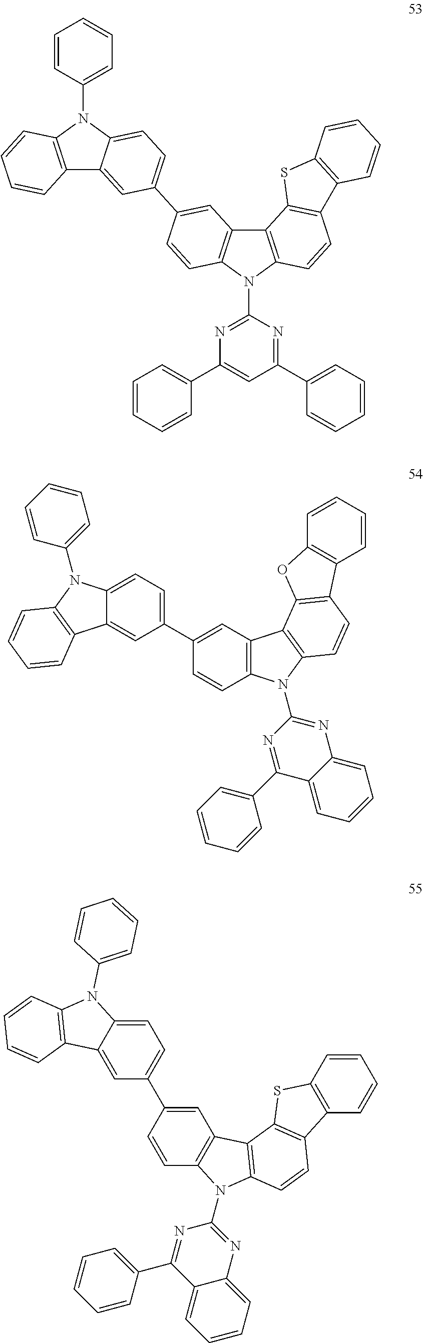 [SMILES: c1ccc(-c2cc(-c3ccccc3)nc(-n3c4ccc(-c5ccc6c(c5)c5ccccc5n6-c5ccccc5)cc4c4c5sc6ccccc6c5ccc43)n2)cc1 ; c1ccc(-c2nc(-n3c4ccc(-c5ccc6c(c5)c5ccccc5n6-c5ccccc5)cc4c4c5oc6ccccc6c5ccc43)nc3ccccc23)cc1 ; c1ccc(-c2nc(-n3c4ccc(-c5ccc6c(c5)c5ccccc5n6-c5ccccc5)cc4c4c5sc6ccccc6c5ccc43)nc3ccccc23)cc1]

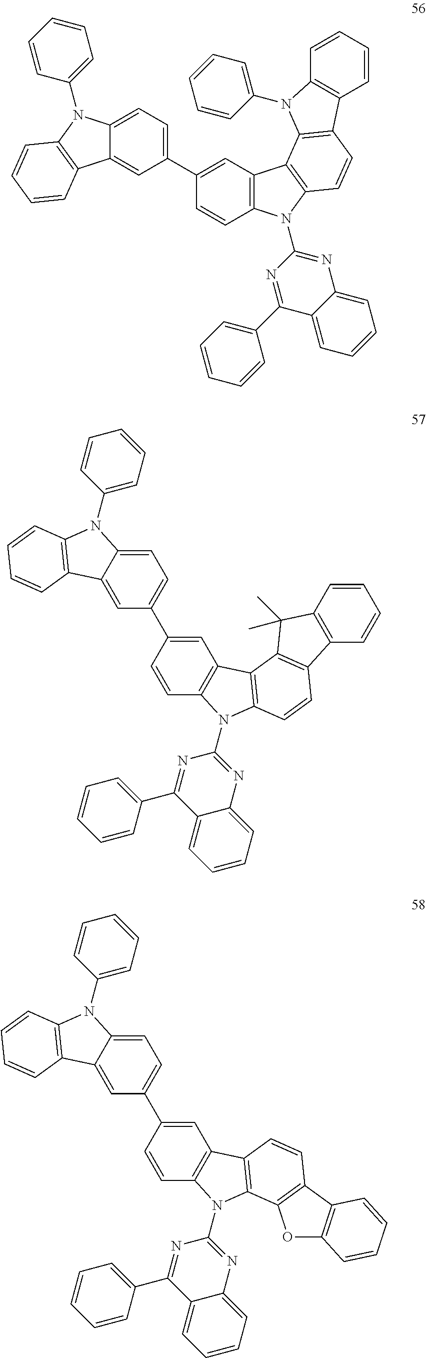 [SMILES: c1ccc(-c2nc(-n3c4ccc(-c5ccc6c(c5)c5ccccc5n6-c5ccccc5)cc4c4c3ccc3c5ccccc5n(-c5ccccc5)c34)nc3ccccc23)cc1 ; CC1(C)c2ccccc2-c2ccc3c(c21)c1cc(-c2ccc4c(c2)c2ccccc2n4-c2ccccc2)ccc1n3-c1nc(-c2ccccc2)c2ccccc2n1 ; c1ccc(-c2nc(-n3c4ccc(-c5ccc6c(c5)c5ccccc5n6-c5ccccc5)cc4c4ccc5c6ccccc6oc5c43)nc3ccccc23)cc1]

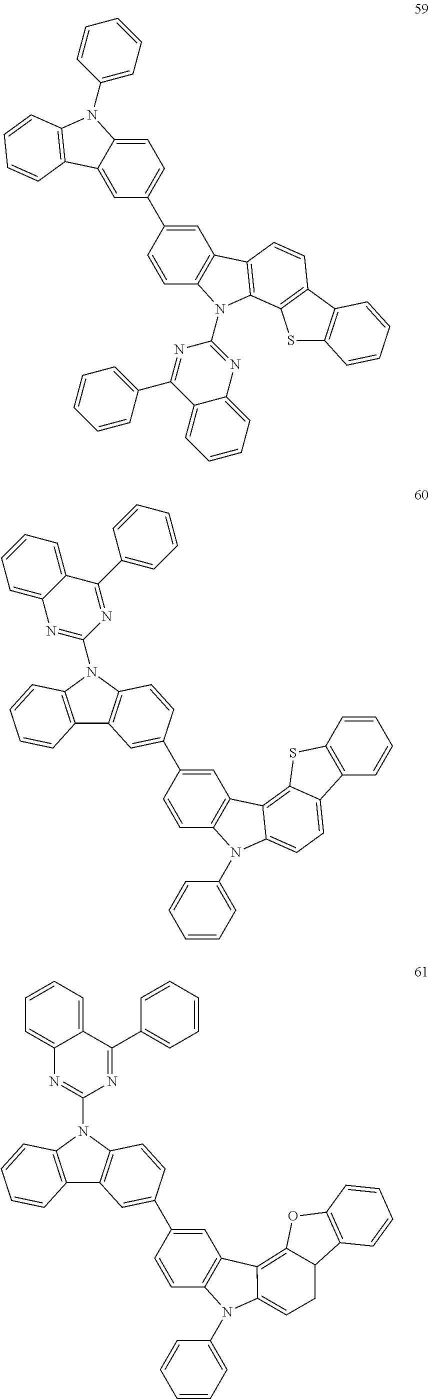

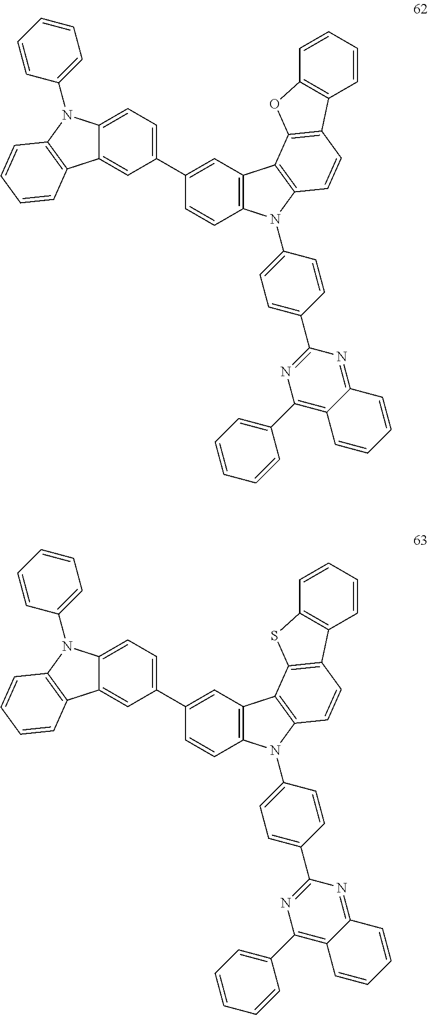 [SMILES: c1ccc(-c2nc(-c3ccc(-n4c5ccc(-c6ccc7c(c6)c6ccccc6n7-c6ccccc6)cc5c5c6oc7ccccc7c6ccc54)cc3)nc3ccccc23)cc1 ; c1ccc(-c2nc(-c3ccc(-n4c5ccc(-c6ccc7c(c6)c6ccccc6n7-c6ccccc6)cc5c5c6sc7ccccc7c6ccc54)cc3)nc3ccccc23)cc1]

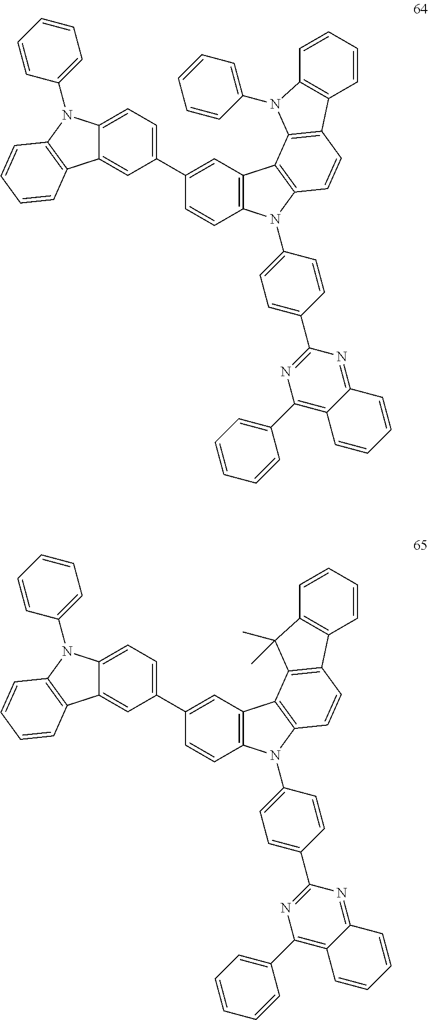 [SMILES: c1ccc(-c2nc(-c3ccc(-n4c5ccc(-c6ccc7c(c6)c6ccccc6n7-c6ccccc6)cc5c5c4ccc4c6ccccc6n(-c6ccccc6)c45)cc3)nc3ccccc23)cc1 ; CC1(C)c2ccccc2-c2ccc3c(c21)c1cc(-c2ccc4c(c2)c2ccccc2n4-c2ccccc2)ccc1n3-c1ccc(-c2nc(-c3ccccc3)c3ccccc3n2)cc1]

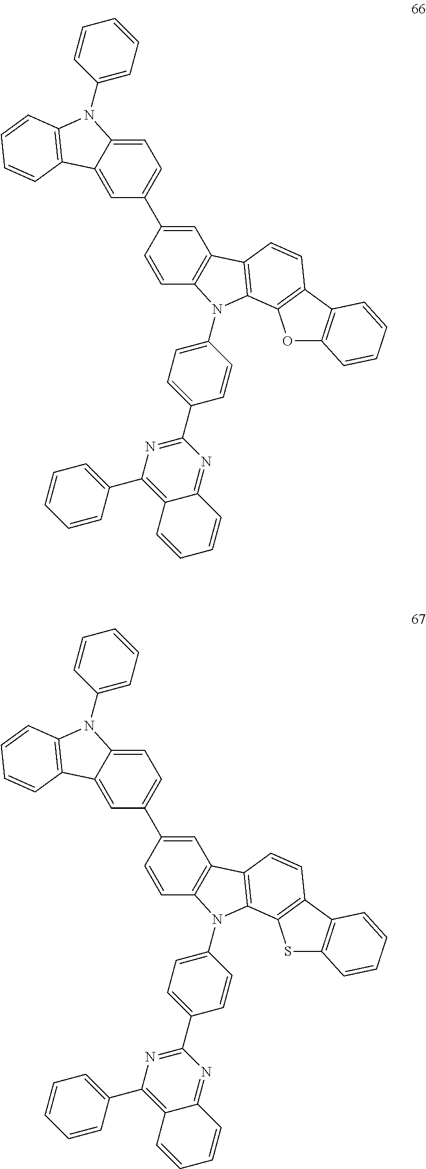 [SMILES: c1ccc(-c2nc(-c3ccc(-n4c5ccc(-c6ccc7c(c6)c6ccccc6n7-c6ccccc6)cc5c5ccc6c7ccccc7oc6c54)cc3)nc3ccccc23)cc1 ; c1ccc(-c2nc(-c3ccc(-n4c5ccc(-c6ccc7c(c6)c6ccccc6n7-c6ccccc6)cc5c5ccc6c7ccccc7sc6c54)cc3)nc3ccccc23)cc1]

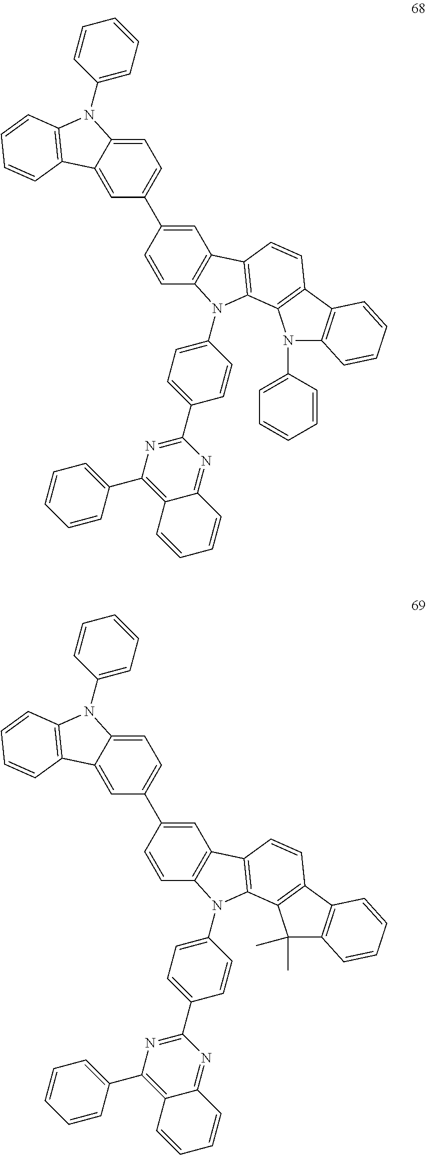 [SMILES: c1ccc(-c2nc(-c3ccc(-n4c5ccc(-c6ccc7c(c6)c6ccccc6n7-c6ccccc6)cc5c5ccc6c7ccccc7n(-c7ccccc7)c6c54)cc3)nc3ccccc23)cc1 ; CC1(C)c2ccccc2-c2ccc3c4cc(-c5ccc6c(c5)c5ccccc5n6-c5ccccc5)ccc4n(-c4ccc(-c5nc(-c6ccccc6)c6ccccc6n5)cc4)c3c21]

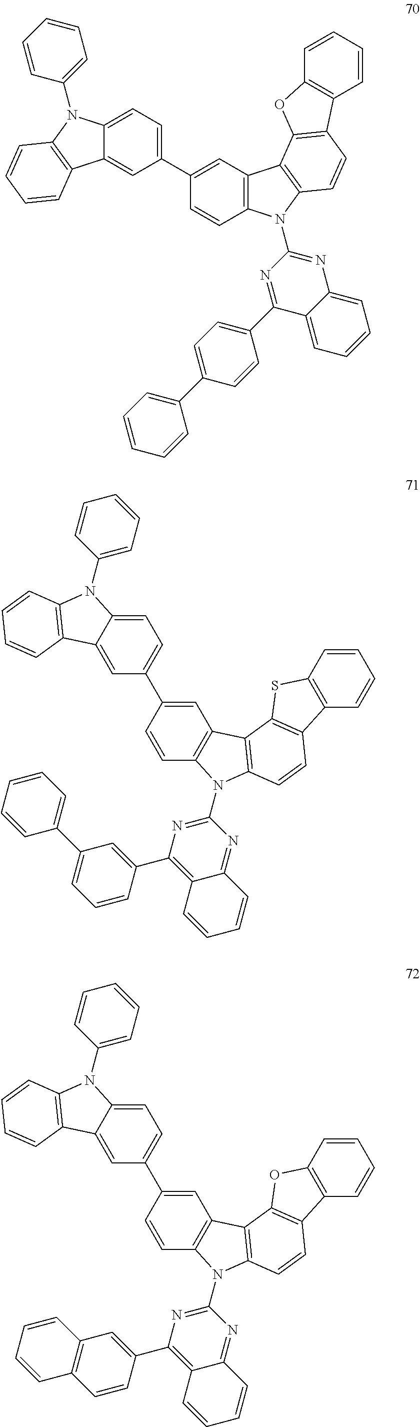 [SMILES: c1ccc(-c2ccc(-c3nc(-n4c5ccc(-c6ccc7c(c6)c6ccccc6n7-c6ccccc6)cc5c5c6oc7ccccc7c6ccc54)nc4ccccc34)cc2)cc1 ; c1ccc(-c2cccc(-c3nc(-n4c5ccc(-c6ccc7c(c6)c6ccccc6n7-c6ccccc6)cc5c5c6sc7ccccc7c6ccc54)nc4ccccc34)c2)cc1 ; c1ccc(-n2c3ccccc3c3cc(-c4ccc5c(c4)c4c6oc7ccccc7c6ccc4n5-c4nc(-c5ccc6ccccc6c5)c5ccccc5n4)ccc32)cc1]

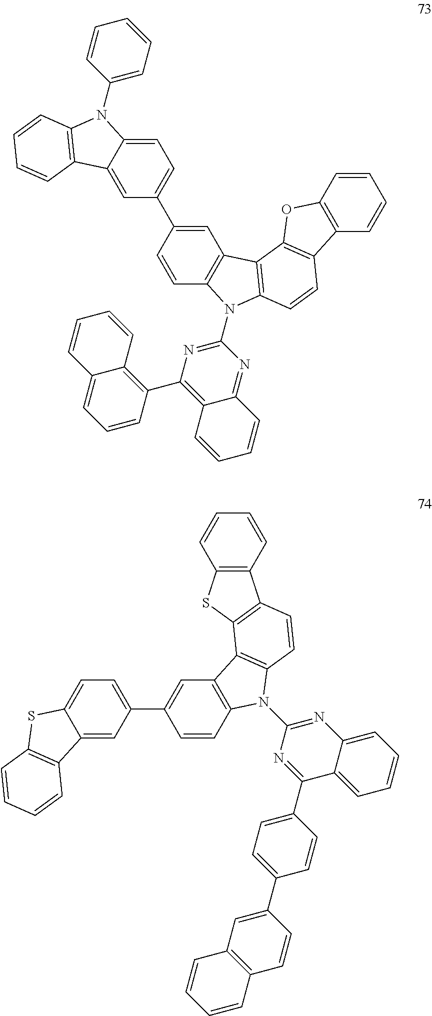 [SMILES: c1ccc(-n2c3ccccc3c3cc(-c4ccc5c(c4)c4c6oc7ccccc7c6ccc4n5-c4nc(-c5cccc6ccccc56)c5ccccc5n4)ccc32)cc1 ; c1ccc2cc(-c3ccc(-c4nc(-n5c6ccc(-c7ccc8sc9ccccc9c8c7)cc6c6c7sc8ccccc8c7ccc65)nc5ccccc45)cc3)ccc2c1]

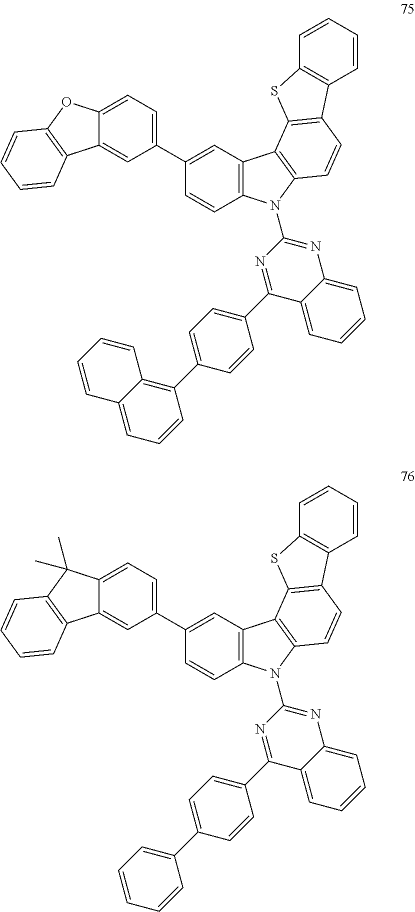 [SMILES: c1ccc2c(-c3ccc(-c4nc(-n5c6ccc(-c7ccc8oc9ccccc9c8c7)cc6c6c7sc8ccccc8c7ccc65)nc5ccccc45)cc3)cccc2c1 ; CC1(C)c2ccccc2-c2cc(-c3ccc4c(c3)c3c5sc6ccccc6c5ccc3n4-c3nc(-c4ccc(-c5ccccc5)cc4)c4ccccc4n3)ccc21]

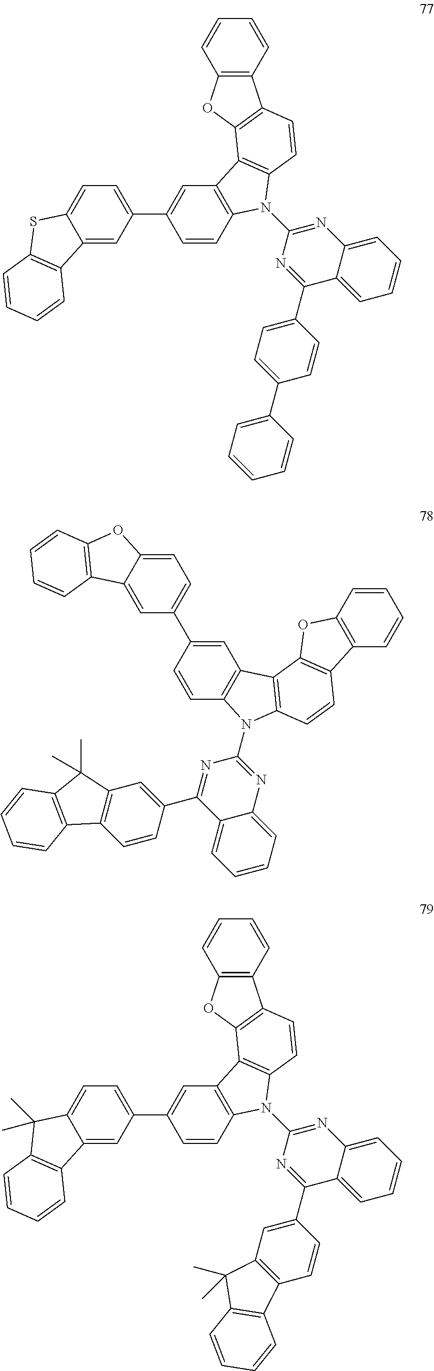

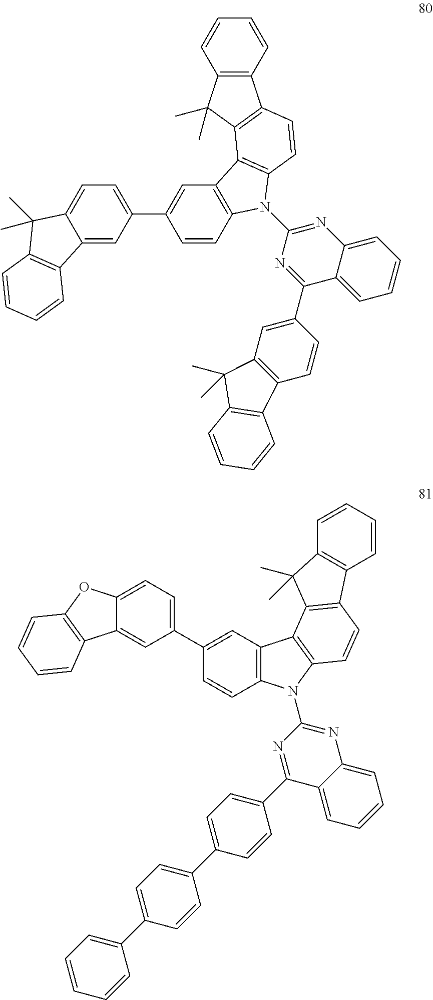 [SMILES: CC1(C)c2ccccc2-c2cc(-c3ccc4c(c3)c3c5c(ccc3n4-c3nc(-c4ccc6c(c4)C(C)(C)c4ccccc4-6)c4ccccc4n3)-c3ccccc3C5(C)C)ccc21 ; CC1(C)c2ccccc2-c2ccc3c(c21)c1cc(-c2ccc4oc5ccccc5c4c2)ccc1n3-c1nc(-c2ccc(-c3ccc(-c4ccccc4)cc3)cc2)c2ccccc2n1]

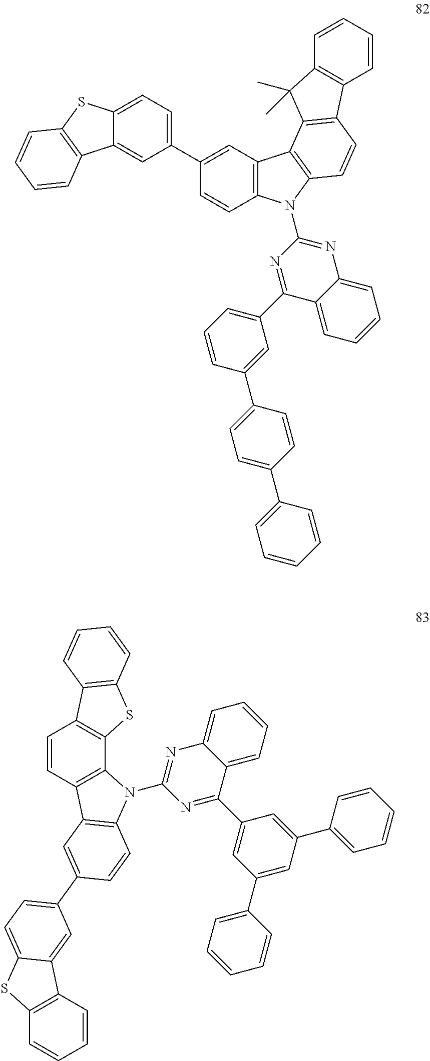 [SMILES: CC1(C)c2ccccc2-c2ccc3c(c21)c1cc(-c2ccc4sc5ccccc5c4c2)ccc1n3-c1nc(-c2cccc(-c3ccc(-c4ccccc4)cc3)c2)c2ccccc2n1 ; c1ccc(-c2cc(-c3ccccc3)cc(-c3nc(-n4c5ccc(-c6ccc7sc8ccccc8c7c6)cc5c5ccc6c7ccccc7sc6c54)nc4ccccc34)c2)cc1]

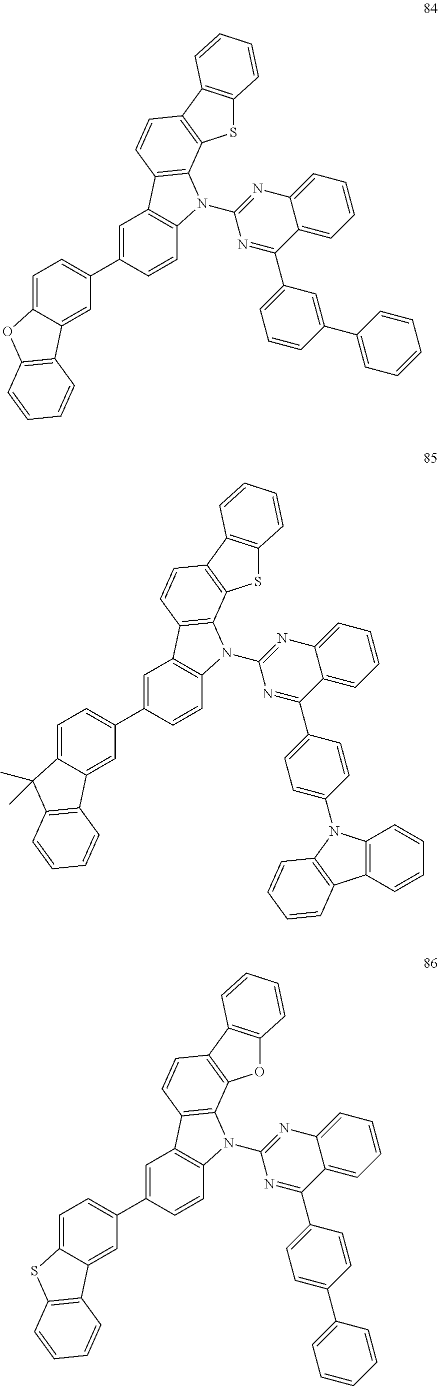 [SMILES: c1ccc(-c2cccc(-c3nc(-n4c5ccc(-c6ccc7oc8ccccc8c7c6)cc5c5ccc6c7ccccc7sc6c54)nc4ccccc34)c2)cc1 ; CC1(C)c2ccccc2-c2cc(-c3ccc4c(c3)c3ccc5c6ccccc6sc5c3n4-c3nc(-c4ccc(-n5c6ccccc6c6ccccc65)cc4)c4ccccc4n3)ccc21 ; c1ccc(-c2ccc(-c3nc(-n4c5ccc(-c6ccc7sc8ccccc8c7c6)cc5c5ccc6c7ccccc7oc6c54)nc4ccccc34)cc2)cc1]

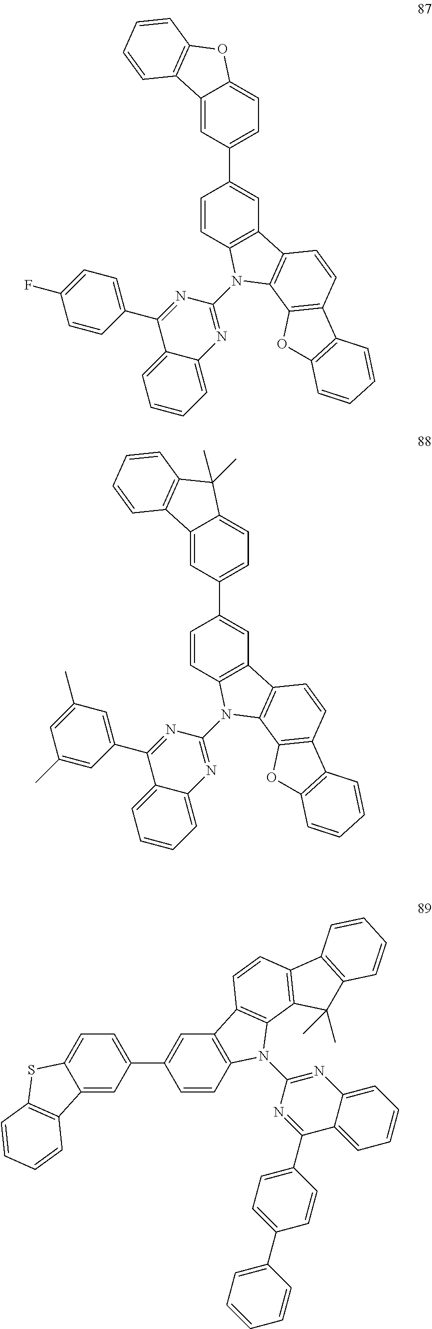

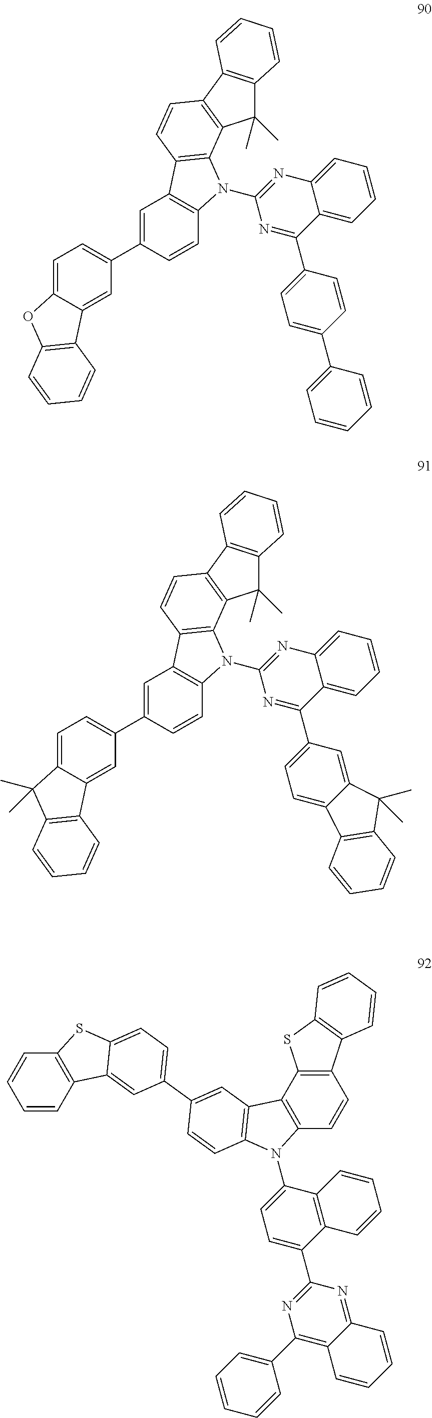

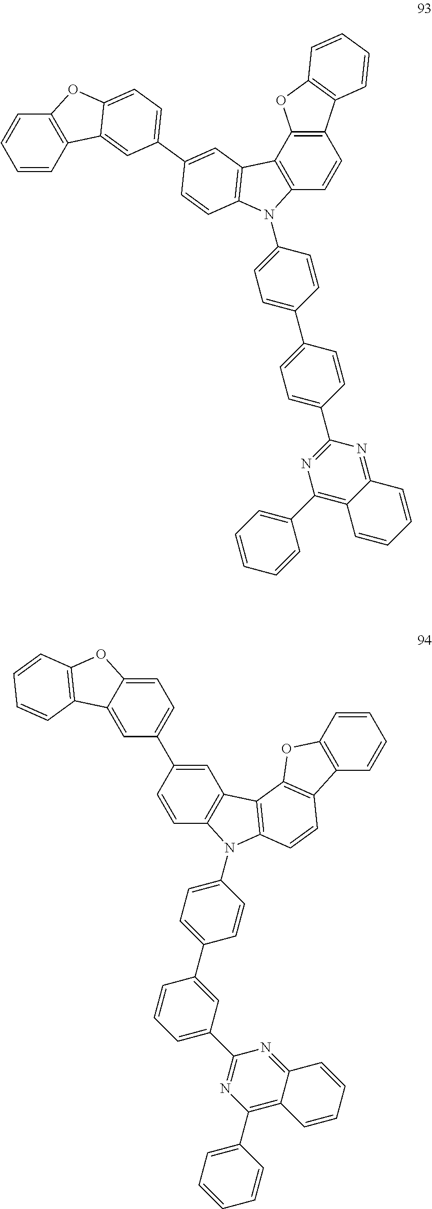 [SMILES: c1ccc(-c2nc(-c3ccc(-c4ccc(-n5c6ccc(-c7ccc8oc9ccccc9c8c7)cc6c6c7oc8ccccc8c7ccc65)cc4)cc3)nc3ccccc23)cc1 ; c1ccc(-c2nc(-c3cccc(-c4ccc(-n5c6ccc(-c7ccc8oc9ccccc9c8c7)cc6c6c7oc8ccccc8c7ccc65)cc4)c3)nc3ccccc23)cc1]

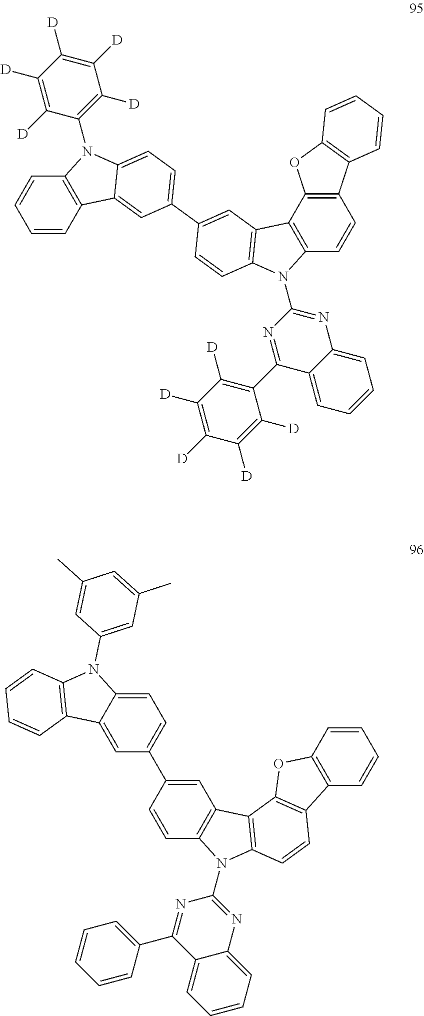 [SMILES: [2H]c1c([2H])c([2H])c(-c2nc(-n3c4ccc(-c5ccc6c(c5)c5ccccc5n6-c5c([2H])c([2H])c([2H])c([2H])c5[2H])cc4c4c5oc6ccccc6c5ccc43)nc3ccccc23)c([2H])c1[2H] ; Cc1cc(C)cc(-n2c3ccccc3c3cc(-c4ccc5c(c4)c4c6oc7ccccc7c6ccc4n5-c4nc(-c5ccccc5)c5ccccc5n4)ccc32)c1]

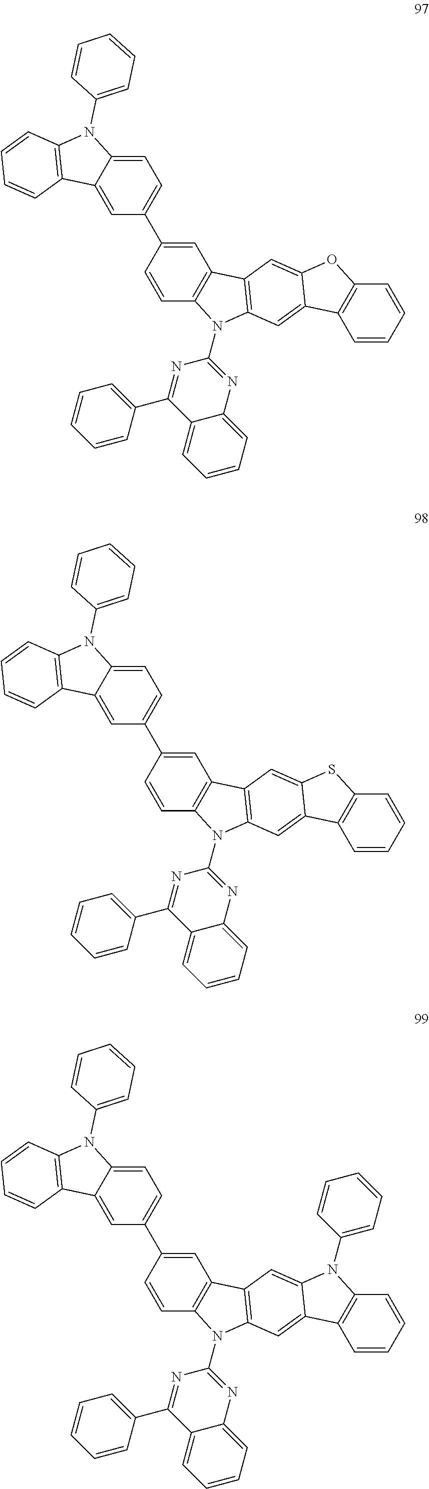 [SMILES: c1ccc(-c2nc(-n3c4ccc(-c5ccc6c(c5)c5ccccc5n6-c5ccccc5)cc4c4cc5oc6ccccc6c5cc43)nc3ccccc23)cc1 ; c1ccc(-c2nc(-n3c4ccc(-c5ccc6c(c5)c5ccccc5n6-c5ccccc5)cc4c4cc5sc6ccccc6c5cc43)nc3ccccc23)cc1 ; c1ccc(-c2nc(-n3c4ccc(-c5ccc6c(c5)c5ccccc5n6-c5ccccc5)cc4c4cc5c(cc43)c3ccccc3n5-c3ccccc3)nc3ccccc23)cc1]

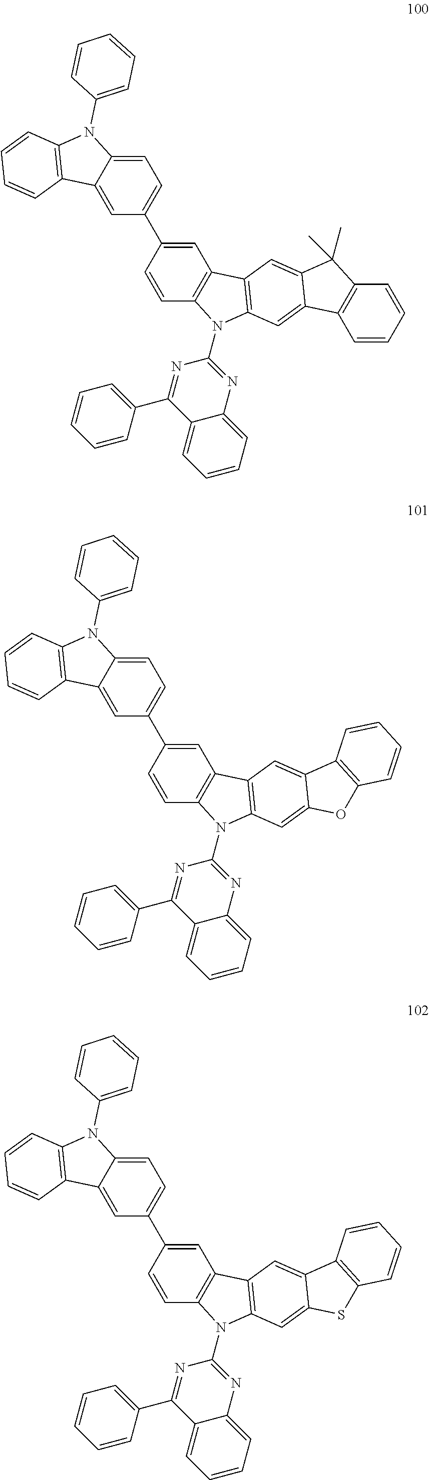 [SMILES: CC1(C)c2ccccc2-c2cc3c(cc21)c1cc(-c2ccc4c(c2)c2ccccc2n4-c2ccccc2)ccc1n3-c1nc(-c2ccccc2)c2ccccc2n1 ; c1ccc(-c2nc(-n3c4ccc(-c5ccc6c(c5)c5ccccc5n6-c5ccccc5)cc4c4cc5c(cc43)oc3ccccc35)nc3ccccc23)cc1 ; c1ccc(-c2nc(-n3c4ccc(-c5ccc6c(c5)c5ccccc5n6-c5ccccc5)cc4c4cc5c(cc43)sc3ccccc35)nc3ccccc23)cc1]

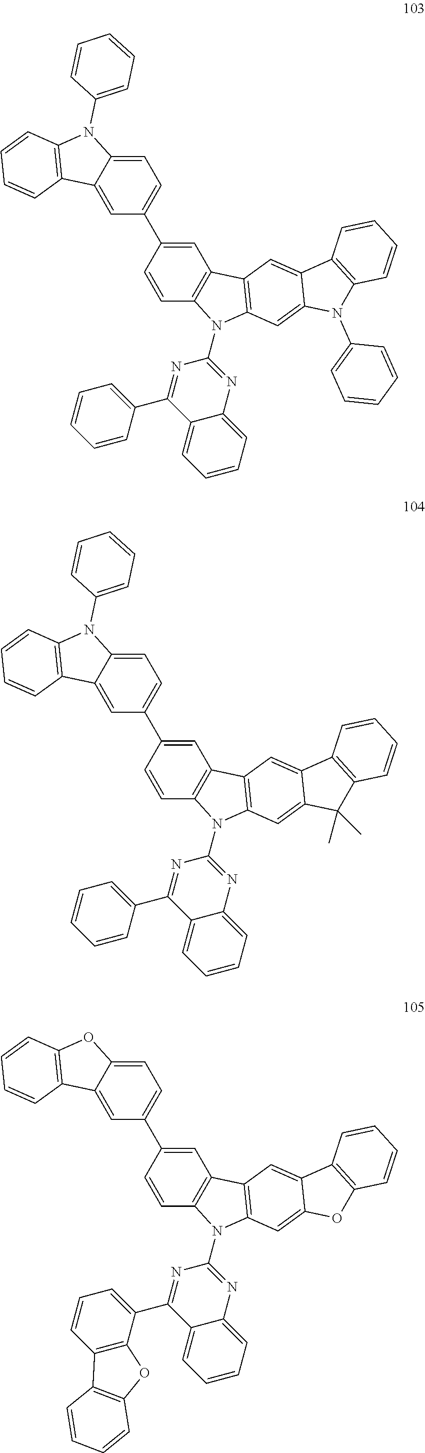 [SMILES: c1ccc(-c2nc(-n3c4ccc(-c5ccc6c(c5)c5ccccc5n6-c5ccccc5)cc4c4cc5c6ccccc6n(-c6ccccc6)c5cc43)nc3ccccc23)cc1 ; CC1(C)c2ccccc2-c2cc3c4cc(-c5ccc6c(c5)c5ccccc5n6-c5ccccc5)ccc4n(-c4nc(-c5ccccc5)c5ccccc5n4)c3cc21 ; c1ccc2c(-c3cccc4c3oc3ccccc34)nc(-n3c4ccc(-c5ccc6oc7ccccc7c6c5)cc4c4cc5c(cc43)oc3ccccc35)nc2c1]

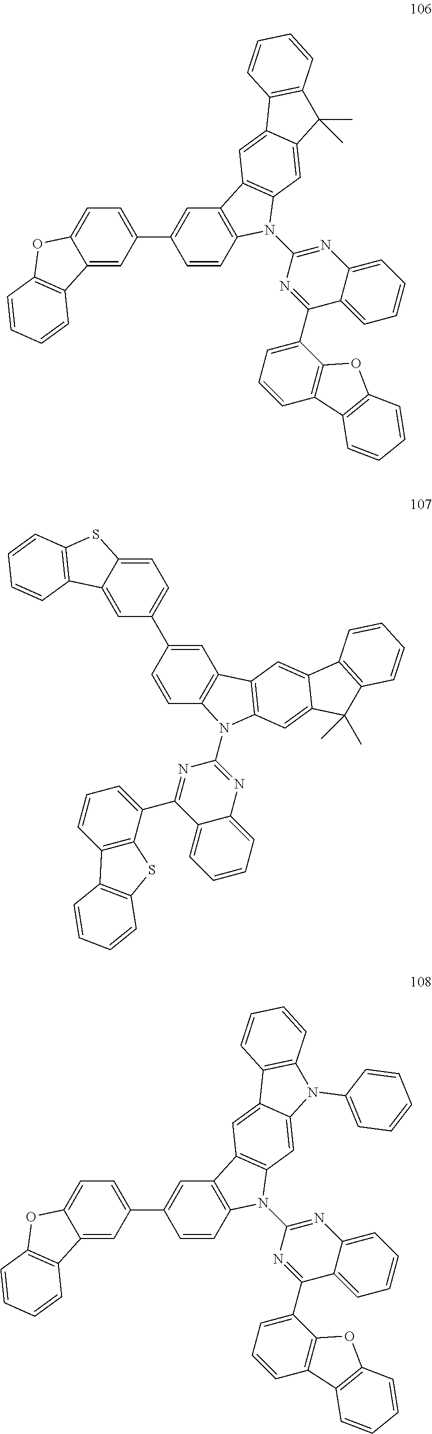 [SMILES: CC1(C)c2ccccc2-c2cc3c4cc(-c5ccc6oc7ccccc7c6c5)ccc4n(-c4nc(-c5cccc6c5oc5ccccc56)c5ccccc5n4)c3cc21 ; CC1(C)c2ccccc2-c2cc3c4cc(-c5ccc6sc7ccccc7c6c5)ccc4n(-c4nc(-c5cccc6c5sc5ccccc56)c5ccccc5n4)c3cc21 ; c1ccc(-n2c3ccccc3c3cc4c5cc(-c6ccc7oc8ccccc8c7c6)ccc5n(-c5nc(-c6cccc7c6oc6ccccc67)c6ccccc6n5)c4cc32)cc1]

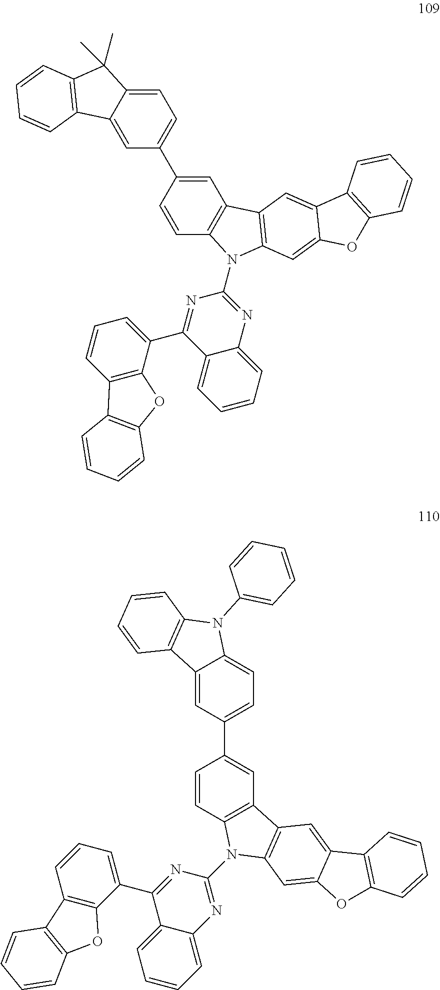 [SMILES: CC1(C)c2ccccc2-c2cc(-c3ccc4c(c3)c3cc5c(cc3n4-c3nc(-c4cccc6c4oc4ccccc46)c4ccccc4n3)oc3ccccc35)ccc21 ; c1ccc(-n2c3ccccc3c3cc(-c4ccc5c(c4)c4cc6c(cc4n5-c4nc(-c5cccc7c5oc5ccccc57)c5ccccc5n4)oc4ccccc46)ccc32)cc1]

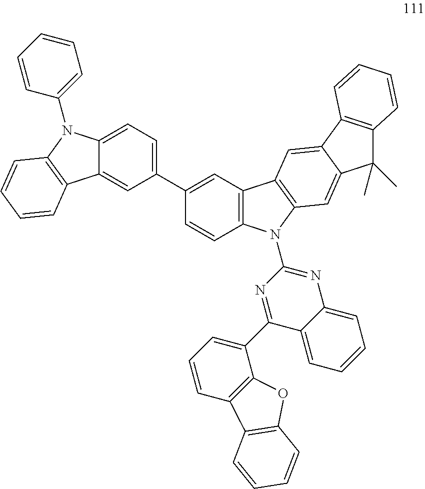 [SMILES: CC1(C)c2ccccc2-c2cc3c4cc(-c5ccc6c(c5)c5ccccc5n6-c5ccccc5)ccc4n(-c4nc(-c5cccc6c5oc5ccccc56)c5ccccc5n4)c3cc21]

6. An organic electroluminescent device, comprising the compound for an organic electronic material of any one of claims 1 to 5.

7. The organic electroluminescent device of claim 6, wherein the organic electroluminescent device comprises: a first electrode; a second electrode; and one or more organic layers interposed between the first electrode and the second electrode, the organic layer includes one or more compounds for an organic electronic material and one or more phosphorescent dopants.

8. The organic electroluminescent device of claim 7, wherein the organic layer further include (A) one or more amine-based compounds selected from the group consisting of arylamine-based compounds or styrylarylamine-based compounds; (B) one or more metals selected from the group consisting of organic metals of Group I, Group II, 4^{th} and 5^{th} period transition metals, lanthanide metals, and d-transition elements or one or more complex compounds including the metals; or a mixture thereof.

9. The organic electroluminescent device of claim 7, wherein the organic layer includes a luminescent layer and a charge generating layer.

10. The organic electroluminescent device of claim 7, wherein the organic layer further includes one or more organic luminescent layers emitting red, green, or blue light to allow white light emission.
